SEQUENCE LISTING (235 sequences)

SEQ ID NO: 1 - protein, 10 aa, Artificial Sequence
  Immunogen
ATYPSGHLDM

SEQ ID NO: 2 - protein, 9 aa, Artificial Sequence
  Control peptide
SRGTHGGFL

SEQ ID NO: 3 - protein, 8 aa, Artificial Sequence
  HCDR1 Consensus
  MISC_FEATURE (1)..(1): Xaa=Gly or Arg
  MISC_FEATURE (5)..(5): Xaa =Ser, Tyr or Asn
  MISC_FEATURE (6)..(6): Xaa=Ser, Gly, Asn or Thr
XFSLXXYA

SEQ ID NO: 4 - protein, 9 aa, Artificial Sequence
  HCDR2 Consensus
  MISC_FEATURE (5)..(5): Xaa=Gly, Ile or Val
  MISC_FEATURE (6)..(6): Xaa=Arg, Val, Ile or Ser
  MISC_FEATURE (7)..(7): Xaa=Ser or Gly
INNDXXXST

SEQ ID NO: 5 - protein, 10 aa, Artificial Sequence
  HCDR3
ARGGTSGYDI

SEQ ID NO: 6 - protein, 7 aa, Artificial Sequence
  LCDR1 Consensus
  MISC_FEATURE (1)..(1): Xaa=Pro or Gln
XSVYKNY

SEQ ID NO: 7 - protein, 3 aa, Artificial Sequence
  LCDR2 Consensus
  MISC_FEATURE (1)..(1): Xaa=Gly or Lys
XAS

SEQ ID NO: 8 - protein, 12 aa, Artificial Sequence
  LCDR3 Consensus
  MISC_FEATURE (5)..(5): Xaa=Arg or Ile
  MISC_FEATURE (7)..(7): Xaa=Ser or Tyr
  MISC_FEATURE (8)..(8): Xaa=Ser, Gly or Arg
  MISC_FEATURE (12)..(12): Xaa=Ala or Ser
AGGYXGXXDIFX

SEQ ID NO: 9 - protein, 8 aa, Artificial Sequence
  HCDR1 Consensus
  MISC_FEATURE (1)..(1): Xaa=Gly or Arg
  MISC_FEATURE (5)..(5): Xaa=Ser, Tyr or Asn
  MISC_FEATURE (6)..(6): Xaa=Ser, Asn or Thr
XFSLXXYA

SEQ ID NO: 10 - protein, 9 aa, Artificial Sequence
  HCDR2 Consensus
  MISC_FEATURE (5)..(5): Xaa=Gly or Val
INNDXISST

SEQ ID NO: 11 - protein, 7 aa, Artificial Sequence
  LCDR1
PSVYKNY

SEQ ID NO: 12 - protein, 3 aa, Artificial Sequence
  LCDR2
GAS

SEQ ID NO: 13 - protein, 12 aa, Artificial Sequence
  LCDR3 Consensus
  MISC_FEATURE (5)..(5): Xaa=Arg or Ile
  MISC_FEATURE (8)..(8): Xaa=Ser or Arg
  MISC_FEATURE (12)..(12): Xaa=Ala or Ser
AGGYXGSXDIFX

SEQ ID NO: 14 - protein, 111 aa, Artificial Sequence
  BC-A9 VL
AQGLTQTPASVSAAVGGTVTINCQSSPSVYKNYLSWYQQKSGQPPKLLIYGASTLASGAPSRFKGSGSGTQFTLTISDVQCDDAATYYCAGGYRGSSDIFAFGGGTEVVVK

SEQ ID NO: 15 - protein, 115 aa, Artificial Sequence
  BC-B10 VH
QSVEESGGRLVTPGAPLTLTCTVSRFSLSSYAMTWVRQAPGKGLEWIGIINNDVISSTWYASWVKGRFTISKTSTTMELKMTSLTTEDTATYFCARGGTSGYDIWGPGTLVTVSS

SEQ ID NO: 16 - protein, 111 aa, Artificial Sequence
  BC-B10 VL
AIKMTQTPSPVSAAVGGTVTINCQSSPSVYKNYLSWYQQKPGQPPKLLIYGASTLASGVPSRFKGSGSGTQFTLTISDVQCDDAATYYCAGGYIGSRDIFSFGGGTEVVVK

SEQ ID NO: 17 - protein, 115 aa, Artificial Sequence
  BC-B4 VH
QSVEESGGRLVTPGGSLTLTCTVSRFSLSSYAMTWVRQAPGKGLEWIGIINNDGISSTWYASWVKGRFTISKTSTTMDLKMTSLTTEDTATYFCARGGTSGYDIWGPGTVVTVSL

SEQ ID NO: 18 - protein, 111 aa, Artificial Sequence
  BC-B4 VL
ALVLTQTPSPVSAAVGGTVTINCQSSQSVYKNYLSWYQQKPGQPPKLLIYGASTLASGVPSRFKGSGSGTQFTLTISDVQCDDAATYYCAGGYRGSGDIFAFGGGTEVVVK

SEQ ID NO: 19 - protein, 115 aa, Artificial Sequence
  BC-B8 VH
QSLEESGGRLVTPGTPLTLTCTVSGFSLYTYAMGWVRQAPGKGLEWIGIINNDGISSTWYASWVKGRFTISKTSTTMDLKMTSLTTEDTATYFCARGGTSGYDIWGPGTLVTVSL

SEQ ID NO: 20 - protein, 111 aa, Artificial Sequence
  BC-B8 VL
AQGLTQTPSPVSAAVGGTVTINCQSGPSVYKNYLSWYQQKAGQPPKLLIYGASTLASGVPSRFKGSGSGTQFTLTISDVQCDDAATYYCAGGYRGSSDIFAFGGGTEVVVK

SEQ ID NO: 21 - protein, 115 aa, Artificial Sequence
  BC-C8 VH
QSLEESGGRLVTPGGSLTLTCTVSRFSLSSYAMTWVRQAPGKGLEWIGIINNDGISSTWYASWVKGRFTISKTSTTMDLKMTSLTTEDTATYFCARGGTSGYDIWGPGTVVTVSL

SEQ ID NO: 22 - protein, 111 aa, Artificial Sequence
  BC-C8 VL
ALVLTQTPSPVSAAVGGTVTINCQSSPSVYKNYLSWYQQKPGQPPKLLIYGASTLASGVPSRFKGSGSGTQFTLTISDVQCDDAATYYCAGGYRGSSDIFAFGGGTEVVVK

SEQ ID NO: 23 - protein, 115 aa, Artificial Sequence; identical to SEQ ID NO: 19
  BC-D3 VH

SEQ ID NO: 24 - protein, 111 aa, Artificial Sequence; identical to SEQ ID NO: 20
  BC-D3 VL

SEQ ID NO: 25 - protein, 115 aa, Artificial Sequence
  BC-E1 VH
QSVKESGGRLVAPGTPLTLTCAVSGFSLSSYAMTWVRQAPGKGLEWIGIINNDGRSSTWYASWVKGRFTISKTSTTMDLKMTSPTTEDTATYFCARGGTSGYDIWGPGTLVTVSL

SEQ ID NO: 26 - protein, 111 aa, Artificial Sequence
  BC-E1 VL
AQGMTQTPSPVSAAVGGTVTINCQSSPSVYKNYLSWYQQKPGQPPKLLIYGASTLASGVPSRFKGSGSGTRFTLTISDVQCDDAATYYCAGGYRGSSDIFAFGGGTEVVVK

SEQ ID NO: 27 - protein, 115 aa, Artificial Sequence; identical to SEQ ID NO: 25
  BC-E11 VH

SEQ ID NO: 28 - protein, 111 aa, Artificial Sequence; identical to SEQ ID NO: 26
  BC-E11 VL

SEQ ID NO: 29 - protein, 116 aa, Artificial Sequence
  BC-E2 VH
QEQLKESGGRLVTPGAPLTLTCTVSRFSLSSYAMTWVRQAPGKGLEWIGIINNDGISSTWYASWVKGRFTISKTSTTMDLKMTSLTTEDTATYFCARGGTSGYDIWGPGTLVTVSL

SEQ ID NO: 30 - protein, 111 aa, Artificial Sequence
  BC-E2 VL
AVVLTQTPSPVSAAVGGTVTINCQSSPSVYKNYLSWYQQKPGQPPKLLIYKASTLASGVPSRFKGSGSGTQFTLTISDVQCDDAATYYCAGGYRGSSDIFAFGGGTEVVVK

SEQ ID NO: 31 - protein, 115 aa, Artificial Sequence; identical to SEQ ID NO: 25
  BC-E3 VH

SEQ ID NO: 32 - protein, 111 aa, Artificial Sequence; identical to SEQ ID NO: 26
  BC-E3 VL

SEQ ID NO: 33 - protein, 115 aa, Artificial Sequence; identical to SEQ ID NO: 25
  BC-E4 VH

SEQ ID NO: 34 - protein, 111 aa, Artificial Sequence; identical to SEQ ID NO: 26
  BC-E4 VL

SEQ ID NO: 35 - protein, 115 aa, Artificial Sequence; identical to SEQ ID NO: 25
  BC-F2 VH

SEQ ID NO: 36 - protein, 111 aa, Artificial Sequence; identical to SEQ ID NO: 26
  BC-F2 VL

SEQ ID NO: 37 - protein, 115 aa, Artificial Sequence
  BC-F4 VH
QSVEESRGRLVTPGGSLTLTCTVSRFSLSNYAMTWVRQAPGKGLEWIGIINNDGISSTWYASWVKGRFTISKTSTTMDLKMTSLTTEDTATYFCARGGTSGYDIWGPGTLVTVSL

SEQ ID NO: 38 - protein, 111 aa, Artificial Sequence
  BC-F4 VL
AQVLTQTPSPVSAAVGGTVTINCQSSPSVYKNYLSWYQQKPGQPPKLLIYGASTLASGVPSRFKGSGSGTQFTLTISDVQCDDAATYYCAGGYRGSSDIFAFGGGTEVVVK

SEQ ID NO: 39 - protein, 115 aa, Artificial Sequence; identical to SEQ ID NO: 17
  BC-F7 VH

SEQ ID NO: 40 - protein, 111 aa, Artificial Sequence; identical to SEQ ID NO: 18
  BC-F7 VL

SEQ ID NO: 41 - protein, 115 aa, Artificial Sequence
  BC-G6 VH
QSVEESGGRLVTPGGSLTLTCTASGFSLSGYAMTWVRQAPGKGLEWIGIINNDGISSTWYASWVKGRFTISKTSTTMDLKMTSLTTEDTATYFCARGGTSGYDIWGPGTLVTVSL

SEQ ID NO: 42 - protein, 111 aa, Artificial Sequence
  BC-G6 VL
ALVMTQTPSPVSAAVGGTVTINCQSSPSVYKNYLSWYQQKPGQPPKLLIYGASTLASGVPSRFKGSGSGTQFTLTISDVQCDDAATYYCAGGYRGSSDIFAFGGGTEVVVK

SEQ ID NO: 43 - protein, 115 aa, Artificial Sequence
  BC-H4 VH
QQLKESGGRLVTPGAPLTLTCTVSRFSLSSYAMTWVRQAPGKGLEWIGIINNDGVSSTWYASWVKGRFTISKTSTTMDLKMTSLTTEDTATYFCARGGTSGYDIWGPGTLVTVSS

SEQ ID NO: 44 - protein, 111 aa, Artificial Sequence
  BC-H4 VL
ALVLTQTPSPVSAAVGGTVTINCQSSPSVYKNYLSWYQQKPGQPPKLLIYGASTLASGVPSRFKGSGSGTQFTLTISDVQCDDAATYYCAGGYRGSGDIFAFGGGTEVVVK

SEQ ID NO: 45 - protein, 115 aa, Artificial Sequence; identical to SEQ ID NO: 19
  BC-H9 VH

SEQ ID NO: 46 - protein, 111 aa, Artificial Sequence; identical to SEQ ID NO: 20
  BC-H9 VL

SEQ ID NO: 47 - protein, 116 aa, Artificial Sequence
  SC-B2 VH
QEQLMESRGRLVTPGGSLTLTCTVSRFSLSNYAMTWVRQAPGKGLEWIGIINNDGISSTWYASWVKGRFTISKTSTTMDLKMTSLTTEDTATYFCARGGTSGYDIWGPGTLVTVSL

SEQ ID NO: 48 - protein, 111 aa, Artificial Sequence
  SC-B2 VL
AQGPTQTPSPVSAAVGGTVTINCQSSPSVYKNYLSWYQQKPGQPPKLLIYGASTLASGVPSRFKGSGSGTQFTLTISDVQCDDAATYYCAGGYRGSGDIFAFGGGTEVVVK

SEQ ID NO: 49 - protein, 115 aa, Artificial Sequence
  SC-B6 VH
QSVKESGGRLVTPGAPLTLTCTVSGFSLNNYAMTWVRQAPGKGLEWIGIINNDGISSTWYASWVKGRFIISKTSTTVDLKMTSLTTEDTATYFCARGGTSGYDIWGPGTLVTVSL

SEQ ID NO: 50 - protein, 111 aa, Artificial Sequence
  SC-B6 VL
AQGPTQTPSPVSAAVGGTVTINCQSSPSVYKNYLSWYQQKPGQPPKLLIYGASTLASGVPSRFKGSRSGTQFTLTISDVQCDDAATYYCAGGYIGSRDIFSFGGGTEVVVK

SEQ ID NO: 51 - protein, 115 aa, Artificial Sequence
  SC-C7 VH
QTVKESGGRLVTPGAPLTLTCTVSRFSLSGYAMTWVRQAPGKGLEWIGIINNDISGSTWYASWVKGRFTISKTSTTMDLKMTSLTTEDTATYFCARGGTSGYDIWGPGTLVTVSS

SEQ ID NO: 52 - protein, 111 aa, Artificial Sequence
  SC-C7 VL
DVVMTQTPSPVSAAVGGTVTINCQSSPSVYKNYLSWYQQKPGQPPKLLIYGASTLASGVPSRFKGSGSGTQFTLTISDVQCDDAATYYCAGGYRGSSDIFAFGGGTEVVVK

SEQ ID NO: 53 - protein, 115 aa, Artificial Sequence
  SC-D1 VH
QSLEESRGRLVTPGGSLTLTCTVSGFSLSSYAMTWVRQAPGKGLEWIGIINNDGVSSTWYASWVKGRFTISKTSTTMDLKMTSLTTEDTATYFCARGGTSGYDIWGPGTLVTVSL

SEQ ID NO: 54 - protein, 111 aa, Artificial Sequence
  SC-D1 VL
DPMMTQTPSPVSAAVGGTVTINCQSSPSVYKNYLSWYQQKPGQPPKLLIYGASTLASGVPSRFKGSGSGTQFTLTISGVQCDDAATYYCAGGYRGSSDIFAFGGGTEVVVK

SEQ ID NO: 55 - protein, 115 aa, Artificial Sequence
  SC-D10 VH
QSVKESGGRLVTPGGSLTLTCTVSRFSLSSYAMTWVRQAPGKGLEWIGIINNDGVSSTWYASWVKGRFTISKTSTTMDLKMTSLTTEDTATYFCARGGTSGYDIWGPGTLVTVSL

SEQ ID NO: 56 - protein, 111 aa, Artificial Sequence
  SC-D10 VL
ALVMTQTPSPVSAAVGGTVTTNCQSSPSVYKNYLSWYQQKPGQPPKLLIYKASTLASGVPSRFSGSGSGTQFTLTISDVQCDDAATYYCAGGYRGSSDIFAFGGGTEVVVK

SEQ ID NO: 57 - protein, 115 aa, Artificial Sequence
  SC-F6 VH
QSVKESGGRLVTPGAPLTLTCTVSRFSLSSYAMTWVRQAPGKGLEWIGIINNDGISSTWYASWVKGRFTISKTSTTMDLKMTSLTTEDTATYFCARGGTSGYDIWGPGTLVTVSL

SEQ ID NO: 58 - protein, 111 aa, Artificial Sequence
  SC-F6 VL
AQGMTQTPASVSAAVGGTVTINCQSSPSVYKNYLSWYQQKSGQPPKLLIYGASTLASGVPSRFKGSGSGTQFTLTISDVQCDDAATYYCAGGYRGSSDIFAFGGGTEVVVK

SEQ ID NO: 59 - protein, 115 aa, Artificial Sequence
  SC-F8 VH
QTVKESGGRLVTPGGSLTLTCTVSRFSLSNYAMTWVRQAPGKGLEWIGIINNDGISSTWYASWVKGRFTISKTSTTMDLKMTSLTTEDTATYFCARGGTSGYDIWGPGTLVTVSL

SEQ ID NO: 60 - protein, 111 aa, Artificial Sequence; identical to SEQ ID NO: 38
  SC-F8 VL

SEQ ID NO: 61 - protein, 115 aa, Artificial Sequence
  SC-G8 VH
QSVEESGGRLVMPGGSLTLTCTASGFSLSSYAMTWVRQAPGKGLEWIGIINNDGSGSTWYASWVKGRFTISKTSTTMDLKMTSLTTEDTATYFCARGGTSGYDIWGPGTLVTVSL

SEQ ID NO: 62 - protein, 111 aa, Artificial Sequence
  SC-G8 VL
AAVLTQTPSPVSAAVGGTVTINCQSSPSVYKNYLSWYQQKPGQPPKLLIYKASTLASGVPSRFKGSGSGTQFTLTISDVQCDDAATYYCAGGYRGYSDIFAFGGGTEVVVK

SEQ ID NO: 63 - protein, 115 aa, Artificial Sequence
  SC-H10 VH
QSLEESGGRLVTPGAPLTLTCTVSRFSLSSYAMTWVRQAPGKGLEWIGIINNDGISSTWYASWVKGRFTISKTSTTMDLKMTSLTTEDTATYFCARGGTSGYDIWGPGTLVTVSL

SEQ ID NO: 64 - protein, 111 aa, Artificial Sequence
  SC-H10 VL
AQGLTQTPSPVSAAVGGTVTINCQSSPSVYKNYLSWYQQKPGQPPKLLIYGASTLASGVPSRFSGSGSGTQFTLTISDVQCDDAATYYCAGGYRGSSDIFAFGGGTEVVVK

SEQ ID NO: 65 - protein, 27 aa, Homo sapiens
ADNQLFVSGPNGTAGIFATYPSGHLDM

SEQ ID NO: 66 - protein, 27 aa, Mus musculus
ANNELFVSGPNGTAGIFATYPSGHLDM

SEQ ID NO: 67 - protein, 10 aa, Homo sapiens
FATYPSGHLD

SEQ ID NO: 68 - protein, 7 aa, Artificial Sequence
  LCDR1
PSVYKNY

SEQ ID NO: 69 - protein, 7 aa, Artificial Sequence
  LCDR1
QSVYKNY

SEQ ID NO: 70 - protein, 3 aa, Artificial Sequence
  LCDR2
GAS

SEQ ID NO: 71 - protein, 3 aa, Artificial Sequence
  LCDR2
KAS

SEQ ID NO: 72 - protein, 12 aa, Artificial Sequence
  LCDR3
AGGYIGSRDIFS

SEQ ID NO: 73 - protein, 12 aa, Artificial Sequence
  LCDR3
AGGYRGSSDIFA

SEQ ID NO: 74 - protein, 12 aa, Artificial Sequence
  LCDR3
AGGYRGSGDIFA

SEQ ID NO: 75 - protein, 12 aa, Artificial Sequence
  LCDR3
AGGYRGYSDIFA

SEQ ID NO: 76 - protein, 8 aa, Artificial Sequence
  HCDR1
RFSLSSYA

SEQ ID NO: 77 - protein, 8 aa, Artificial Sequence
  HCDR1
GFSLYTYA

SEQ ID NO: 78 - protein, 8 aa, Artificial Sequence
  HCDR1
GFSLNNYA

SEQ ID NO: 79 - protein, 8 aa, Artificial Sequence
  HCDR1
RFSLSNYA

SEQ ID NO: 80 - protein, 8 aa, Artificial Sequence
  HCDR1
GFSLSSYA

SEQ ID NO: 81 - protein, 8 aa, Artificial Sequence
  HCDR1
GFSLSGYA

SEQ ID NO: 82 - protein, 8 aa, Artificial Sequence
  HCDR1
RFSLSGYA

SEQ ID NO: 83 - protein, 9 aa, Artificial Sequence
  HCDR2
INNDVISST

SEQ ID NO: 84 - protein, 9 aa, Artificial Sequence
  HCDR2
INNDGISST

SEQ ID NO: 85 - protein, 9 aa, Artificial Sequence
  HCDR2
INNDGRSST

SEQ ID NO: 86 - protein, 9 aa, Artificial Sequence
  HCDR2
INNDGVSST

SEQ ID NO: 87 - protein, 9 aa, Artificial Sequence
  HCDR2
INNDGSSST

SEQ ID NO: 88 - protein, 9 aa, Artificial Sequence
  HCDR2
INNDISGST

SEQ ID NO: 89 - protein, 9 aa, Artificial Sequence
  HCDR2
INNDGSGST

SEQ ID NO: 90 - protein, 5 aa, Homo sapiens
YPSGH

SEQ ID NO: 91 - protein, 5 aa, Homo sapiens
GHLDM

SEQ ID NO: 92 - protein, 5 aa, Homo sapiens
TAGIF

SEQ ID NO: 93 - protein, 2 aa, Homo sapiens
PS

SEQ ID NO: 94 - protein, 15 aa, Homo sapiens
SGPNGTAGIFATYPS

SEQ ID NO: 95 - protein, 115 aa, Artificial Sequence
  BC-A3 VH
QTVKESGGRLVTPGAPLTLTCTVSRFSLSSYAMTWVRQAPGKGLEWIGIINNDGVSSTWYASWVKGRFTISKTSTTMDLKMTSLTTEDTATYFCARGGTSGYDIWGPGTLVTVSL

SEQ ID NO: 96 - protein, 9 aa, Homo sapiens
TYPSGHLDM

SEQ ID NO: 97 - protein, 8 aa, Homo sapiens
YPSGHLDM

SEQ ID NO: 98 - protein, 7 aa, Homo sapiens
PSGHLDM

SEQ ID NO: 99 - protein, 6 aa, Homo sapiens
SGHLDM

SEQ ID NO: 100 - protein, 5 aa, Homo sapiens
GHLDM

SEQ ID NO: 101 - protein, 9 aa, Homo sapiens
ATYPSGHLD

SEQ ID NO: 102 - protein, 8 aa, Homo sapiens
ATYPSGHL

SEQ ID NO: 103 - protein, 7 aa, Homo sapiens
ATYPSGH

SEQ ID NO: 104 - protein, 6 aa, Homo sapiens
ATYPSG

SEQ ID NO: 105 - protein, 15 aa, Homo sapiens
TAGIFATYPSGHLDM

SEQ ID NO: 106 - protein, 14 aa, Homo sapiens
AGIFATYPSGHLDM

SEQ ID NO: 107 - protein, 13 aa, Homo sapiens
GIFATYPSGHLDM

SEQ ID NO: 108 - protein, 12 aa, Homo sapiens
IFATYPSGHLDM

SEQ ID NO: 109 - protein, 11 aa, Homo sapiens
FATYPSGHLDM

SEQ ID NO: 110 - protein, 20 aa, Homo sapiens
SGPNGTAGIFATYPSGHLDM

SEQ ID NO: 111 - RNA, 19 nt, Artificial Sequence
  mouse AQP3 siRNA
ucguugacccuuauaacaa

SEQ ID NO: 112 - RNA, 19 nt, Artificial Sequence
  mouse AQP3 siRNA
gggcuucaauucuggcuau

SEQ ID NO: 113 - RNA, 19 nt, Artificial Sequence
  mouse AQP3 siRNA
cauuaggcgaugugagguu

SEQ ID NO: 114 - RNA, 19 nt, Artificial Sequence
  mouse AQP3 siRNA
gcugaaguccaggucguaa

SEQ ID NO: 115 - RNA, 19 nt, Artificial Sequence
  non-targeting siRNA
ugguuuacaugucgacuaa

SEQ ID NO: 116 - RNA, 19 nt, Artificial Sequence
  non-targeting siRNA
ugguuuacauguuguguga

SEQ ID NO: 117 - RNA, 19 nt, Artificial Sequence
  non-targeting siRNA
ugguuuacauguuuucuga

SEQ ID NO: 118 - RNA, 19 nt, Artificial Sequence
  non-targeting siRNA
ugguuuacauguuuuccua

SEQ ID NO: 119 - protein, 111 aa, Artificial Sequence
  BC-A3 VL
AQVLTQTPASVSAAVRGTVTINCQSSPSVYKNYLSWYQQKSGQPPKLLIYGASTLASGVPSRFKGSGSGTQFTLTISDVQCDDAATYYCAGGYRGSSDIFAFGGGTEVVVK

SEQ ID NO: 120 - protein, 115 aa, Artificial Sequence
  BC-A8 VH
QTVKESGGRLVTPGAPLTLTCTVSRFSLSSYAMTWVRQAPGKGLEWIGIINNDGSSSTWYASWVKGRFTISKTSTTMDLKMTSLTTEDTATYFCARGGTSGYDIWGPGTLVTVSL

SEQ ID NO: 121 - protein, 111 aa, Artificial Sequence
  BC-A8 VL
AQGLTQTPSPVSAAVGGTVTINCQSSPSVYKNYLSWYQQKPGQPPKLLIYGASTLASGVPSRFKGSGSGTQFTLTISDVQCDDAATYYCAGGYRGSSDIFAFGGGTEVVVK

SEQ ID NO: 122 - protein, 115 aa, Artificial Sequence; identical to SEQ ID NO: 17
  BC-A9 VH

SEQ ID NO: 123 - protein, 449 aa, Artificial Sequence
  Antibody A HC
QVQLQQPGAELVRPGASVTLSCKASGYTFTDYEMHWVKQTPVHGLEWIGGVDPETGGTGYNQKFRGKAILTADKSSSTAYMELRSLTSEDSAVYYCARHGGSFYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 124 - protein, 214 aa, Artificial Sequence
  Antibody A LC
DIVMTQSPKFMSTSVGDRVSITCKASQDVSTAVAWYQQKPGQSPKLLIYWASTRHTGVPDRFTGSGSGTDYTLTISSVQAEDLALYYCQQHYSTPPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 125 - protein, 454 aa, Artificial Sequence
  Antibody B HC
EVQLVESGGDLVKPGGSLKLSCAASGFTFSSYGMSWVRQTPDKRLEWVATISRGSIYTYYPDSVKGRFTISRDNAKNTLYLQMSSLKSEDTAMYYCARLSLYDYDGARYTMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 126 - protein, 213 aa, Artificial Sequence
  Antibody B LC
DIVMTQSPKFMSTSVGDRVSITCKASQDVGTAVAWYQQKPGQSPKLLIYWASTRHTGVPDRFTGSGSGTDFTLTISNVQSEDLADYFCQQYSSYHTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 127 - protein, 451 aa, Artificial Sequence
  Antibody C HC
QVQLKQSGAELARPGASVKLSCKASGYNFKSYGISWVKQRTGQGLEWIGEIYPGSGNTYYNEKLKGKATLTADKSSSTAYMELRSLTSEDSAVYFCARTYGYDSFPWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 128 - protein, 219 aa, Artificial Sequence
  Antibody C LC
DIVMTQAAPSVPVTPGESVSISCRSSKSLLHSNGNTYLYWFLQRPGQSPQLLIYRVSNLASGVPDRFSGSGSGTAFTLRISRVEAEDEGVYYCMQHLEYPFTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 129 - protein, 447 aa, Artificial Sequence
  Antibody D HC
EVQLQQSGAELVRPGASVTLSCKASGYTFTDYEMHWVQQTPVHGLEWIGGIDPETGGTGYNQKFKGKAILTADKSSSTAYMELRSLTSEDSAVYFCTRHGSYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 130 - protein, 214 aa, Artificial Sequence
  Antibody D LC
DIVMTQSPKFMSTSVGDRVSITCKASQDVSTAVAWYQQKPGQSPKLLIYWASTRHTGVPDRFTGSGSGTDYTLTISSVQAEDLALYYCQQHYSTPPTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 131 - protein, 450 aa, Artificial Sequence
  Antibody E HC
EVKLLESGAELVRPGASVTLSCKASGYTFTDYEMHWVKQTPVHGLEWIGGIDPESGGTGYNQKFKGKAILTADKSSSTAYMELRSLTSEDSAVYFCTRSGYYGSPLLDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 132 - protein, 215 aa, Artificial Sequence
  Antibody E LC
QIVLSQSPAIMSASLGERVTMTCTASSSVSSSYLHWYQQKPGSSPKLWIYSTSNLASGVPARFSGSGSGTSYSLTISSMEAEDAATYYCHQYHRSPPTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 133 - protein, 449 aa, Artificial Sequence
  Antibody F HC
QVQLKESGPELVKPGASVKISCKASGYTFTDYYINWVKQRPGQGLEWIGWIFPGSGSTYYNEKFKGKATLTVDKSSSTAYMLLSSLTSEDSAVYFCADYGSSYRYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 134 - protein, 213 aa, Artificial Sequence
  Antibody F LC
DIVMTQSPAILSASPGEKVTMTCRASSSVSYMHWYQQKPGSSPKPWIYATSYLASGVPARFSGSGSGTSYSLTIGRVEAEDAATYYCQQWSSNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 135 - protein, 454 aa, Artificial Sequence
  Antibody G HC
QVQLKQSGAELVRPGASVTLSCKASGYTFTDYEMHWVKQTPVHGLEWIGGIDPETGGTAYNQKFKGKAILTADKSSSTAYMELRSLTSEDSAVYYCTRWGAITSFVALRGFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 136 - protein, 220 aa, Artificial Sequence
  Antibody G LC
DIQMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 137 - protein, 454 aa, Artificial Sequence
  Antibody H HC
EVKLVESGGDLVKPGGSLKLSCAASGFTFSSYGMSWVRQTPDKRLEWVATISRRSIYTYYPDSVQGRFTISRDNAKNTLYLQMSSLKSEDTAMYYCARLSLYDYDGARYTMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 138 - protein, 213 aa, Artificial Sequence
  Antibody H LC
DIKMTQSPKFMSTSVGDRVSITCKASQDVGTAVAWYQQKPGQSPKLLIYWASTRHTGVPDRFTGSGSGTDFTLTISNVQSEDLADYFCQQYSSYHTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 139 - protein, 454 aa, Artificial Sequence
  Antibody J HC
QVHLQQSGTELVKPGASVKLSCEASGYTFTSYWMHWVKQRPGQGLEWIGNINPSNGGTNYNEKFKSKATLTVDKSSSTAYMQLSSLTSEDSAVYYCARGGIYYGNYDYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 140 - protein, 219 aa, Artificial Sequence
  Antibody J LC
DIVMTQAAPSVPVTPGESVSISCRSSKSLLHSNGNTYLYWFLQRPGQSPQLLIYRVSNLASGVPDRFSGSGSGTAFTLRISRVEAEDVGVYYCMQHLEYPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 141 - protein, 448 aa, Artificial Sequence
  Antibody K HC
QVQLKQSGAELVRPGTSVKVSCKASGYAFTNYLIEWVKQRPGQGLEWIGVINPGSGGTNYNEKFKGKATLTADKSSSTAYMQLSSLTSEDSAVYFCARWGFYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 142 - protein, 214 aa, Artificial Sequence
  Antibody K LC
DIVMTQSPSSLSASLGERVSLTCRASQEISGYLSWLQQKPDGTIKRLIYAASTLDSGVPKRFSGSRSGSDYSLTISSLESEDFADYYCLQYASYPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 143 - protein, 8 aa, Artificial Sequence
  Antibody A HCDR1 amino acid sequence (IMGT definition)
GYTFTDYE

SEQ ID NO: 144 - protein, 8 aa, Artificial Sequence
  Antibody A HCDR2 amino acid sequence (IMGT definition)
VDPETGGT

SEQ ID NO: 145 - protein, 12 aa, Artificial Sequence
  Antibody A HCDR3 amino acid sequence (IMGT definition)
ARHGGSFYAMDY

SEQ ID NO: 146 - protein, 6 aa, Artificial Sequence
  Antibody A LCDR1 amino acid sequence (IMGT definition)
QDVSTA

SEQ ID NO: 147 - protein, 3 aa, Artificial Sequence
  Antibody A LCDR2 amino acid sequence (IMGT definition)
WAS

SEQ ID NO: 148 - protein, 9 aa, Artificial Sequence
  Antibody A LCDR3 amino acid sequence (IMGT definition)
QQHYSTPPT

SEQ ID NO: 149 - protein, 119 aa, Artificial Sequence
  Antibody A VH amino acid sequence (predicted mature)
QVQLQQPGAELVRPGASVTLSCKASGYTFTDYEMHWVKQTPVHGLEWIGGVDPETGGTGYNQKFRGKAILTADKSSSTAYMELRSLTSEDSAVYYCARHGGSFYAMDYWGQGTSVTVSS

SEQ ID NO: 150 - protein, 119 aa, Artificial Sequence; identical to SEQ ID NO: 149
  Antibody A VL amino acid sequence (predicted mature)

SEQ ID NO: 151 - protein, 8 aa, Artificial Sequence
  Antibody B HCDR1 amino acid sequence (IMGT definition)
GFTFSSYG

SEQ ID NO: 152 - protein, 8 aa, Artificial Sequence
  Antibody B HCDR2 amino acid sequence (IMGT definition)
ISRGSIYT

SEQ ID NO: 153 - protein, 17 aa, Artificial Sequence
  Antibody B HCDR3 amino acid sequence (IMGT definition)
ARLSLYDYDGARYTMDY

SEQ ID NO: 154 - protein, 6 aa, Artificial Sequence
  Antibody B LCDR1 amino acid sequence (IMGT definition)
QDVGTA

SEQ ID NO: 155 - protein, 3 aa, Artificial Sequence
  Antibody B LCDR2 amino acid sequence (IMGT definition)
WAS

SEQ ID NO: 156 - protein, 8 aa, Artificial Sequence
  Antibody B LCDR3 amino acid sequence (IMGT definition)
QQYSSYHT

SEQ ID NO: 157 - protein, 124 aa, Artificial Sequence
  Antibody B VH amino acid sequence (predicted mature)
EVQLVESGGDLVKPGGSLKLSCAASGFTFSSYGMSWVRQTPDKRLEWVATISRGSIYTYYPDSVKGRFTISRDNAKNTLYLQMSSLKSEDTAMYYCARLSLYDYDGARYTMDYWGQGTSVTVSS

SEQ ID NO: 158 - protein, 106 aa, Artificial Sequence
  Antibody B VL amino acid sequence (predicted mature)
DIVMTQSPKFMSTSVGDRVSITCKASQDVGTAVAWYQQKPGQSPKLLIYWASTRHTGVPDRFTGSGSGTDFTLTISNVQSEDLADYFCQQYSSYHTFGAGTKLELK

SEQ ID NO: 159 - protein, 8 aa, Artificial Sequence
  Antibody C HCDR1 amino acid sequence (IMGT definition)
GYNFKSYG

SEQ ID NO: 160 - protein, 8 aa, Artificial Sequence
  Antibody C HCDR2 amino acid sequence (IMGT definition)
IYPGSGNT

SEQ ID NO: 161 - protein, 14 aa, Artificial Sequence
  Antibody C HCDR3 amino acid sequence (IMGT definition)
ARTYGYDSFPWFAY

SEQ ID NO: 162 - protein, 11 aa, Artificial Sequence
  Antibody C LCDR1 amino acid sequence (IMGT definition)
KSLLHSNGNTY

SEQ ID NO: 163 - protein, 3 aa, Artificial Sequence
  Antibody C LCDR2 amino acid sequence (IMGT definition)
RVS

SEQ ID NO: 164 - protein, 9 aa, Artificial Sequence
  Antibody C LCDR3 amino acid sequence (IMGT definition)
MQHLEYPFT

SEQ ID NO: 165 - protein, 121 aa, Artificial Sequence
  Antibody C VH amino acid sequence (predicted mature)
QVQLKQSGAELARPGASVKLSCKASGYNFKSYGISWVKQRTGQGLEWIGEIYPGSGNTYYNEKLKGKATLTADKSSSTAYMELRSLTSEDSAVYFCARTYGYDSFPWFAYWGQGTLVTVSS

SEQ ID NO: 166 - protein, 112 aa, Artificial Sequence
  Antibody C VL amino acid sequence (predicted mature)
DIVMTQAAPSVPVTPGESVSISCRSSKSLLHSNGNTYLYWFLQRPGQSPQLLIYRVSNLASGVPDRFSGSGSGTAFTLRISRVEAEDEGVYYCMQHLEYPFTFGAGTKLEIK

SEQ ID NO: 167 - protein, 8 aa, Artificial Sequence
  Antibody D HCDR1 amino acid sequence (IMGT definition)
GYTFTDYE

SEQ ID NO: 168 - protein, 8 aa, Artificial Sequence
  Antibody D HCDR2 amino acid sequence (IMGT definition)
IDPETGGT

SEQ ID NO: 169 - protein, 10 aa, Artificial Sequence
  Antibody D HCDR3 amino acid sequence (IMGT definition)
TRHGSYAMDY

SEQ ID NO: 170 - protein, 6 aa, Artificial Sequence
  Antibody D LCDR1 amino acid sequence (IMGT definition)
QDVSTA

SEQ ID NO: 171 - protein, 3 aa, Artificial Sequence
  Antibody D LCDR2 amino acid sequence (IMGT definition)
WAS

SEQ ID NO: 172 - protein, 9 aa, Artificial Sequence
  Antibody D LCDR3 amino acid sequence (IMGT definition)
QQHYSTPPT

SEQ ID NO: 173 - protein, 117 aa, Artificial Sequence
  Antibody D VH amino acid sequence (predicted mature)
EVQLQQSGAELVRPGASVTLSCKASGYTFTDYEMHWVQQTPVHGLEWIGGIDPETGGTGYNQKFKGKAILTADKSSSTAYMELRSLTSEDSAVYFCTRHGSYAMDYWGQGTSVTVSS

SEQ ID NO: 174 - protein, 107 aa, Artificial Sequence
  Antibody D VL amino acid sequence (predicted mature)
DIVMTQSPKFMSTSVGDRVSITCKASQDVSTAVAWYQQKPGQSPKLLIYWASTRHTGVPDRFTGSGSGTDYTLTISSVQAEDLALYYCQQHYSTPPTFGGGTRLEIK

SEQ ID NO: 175 - protein, 8 aa, Artificial Sequence
  Antibody E HCDR1 amino acid sequence (IMGT definition)
GYTFTDYE

SEQ ID NO: 176 - protein, 8 aa, Artificial Sequence
  Antibody E HCDR2 amino acid sequence (IMGT definition)
IDPESGGT

SEQ ID NO: 177 - protein, 13 aa, Artificial Sequence
  Antibody E HCDR3 amino acid sequence (IMGT definition)
TRSGYYGSPLLDY

SEQ ID NO: 178 - protein, 7 aa, Artificial Sequence
  Antibody E LCDR1 amino acid sequence (IMGT definition)
SSVSSSY

SEQ ID NO: 179 - protein, 3 aa, Artificial Sequence
  Antibody E LCDR2 amino acid sequence (IMGT definition)
STS

SEQ ID NO: 180 - protein, 9 aa, Artificial Sequence
  Antibody E LCDR3 amino acid sequence (IMGT definition)
HQYHRSPPT

SEQ ID NO: 181 - protein, 120 aa, Artificial Sequence
  Antibody E VH amino acid sequence (predicted mature)
EVKLLESGAELVRPGASVTLSCKASGYTFTDYEMHWVKQTPVHGLEWIGGIDPESGGTGYNQKFKGKAILTADKSSSTAYMELRSLTSEDSAVYFCTRSGYYGSPLLDYWGQGTTLTVSS

SEQ ID NO: 182 - protein, 108 aa, Artificial Sequence
  Antibody E VL amino acid sequence (predicted mature)
QIVLSQSPAIMSASLGERVTMTCTASSSVSSSYLHWYQQKPGSSPKLWIYSTSNLASGVPARFSGSGSGTSYSLTISSMEAEDAATYYCHQYHRSPPTFGAGTKLEIK

SEQ ID NO: 183 - protein, 8 aa, Artificial Sequence
  Antibody F HCDR1 amino acid sequence (IMGT definition)
GYTFTDYY

SEQ ID NO: 184 - protein, 8 aa, Artificial Sequence
  Antibody F HCDR2 amino acid sequence (IMGT definition)
IFPGSGST

SEQ ID NO: 185 - protein, 12 aa, Artificial Sequence
  Antibody F HCDR3 amino acid sequence (IMGT definition)
ADYGSSYRYFDV

SEQ ID NO: 186 - protein, 5 aa, Artificial Sequence
  Antibody F LCDR1 amino acid sequence (IMGT definition)
SSVSY

SEQ ID NO: 187 - protein, 3 aa, Artificial Sequence
  Antibody F LCDR2 amino acid sequence (IMGT definition)
ATS

SEQ ID NO: 188 - protein, 9 aa, Artificial Sequence
  Antibody F LCDR3 amino acid sequence (IMGT definition)
QQWSSNPLT

SEQ ID NO: 189 - protein, 119 aa, Artificial Sequence
  Antibody F VH amino acid sequence (predicted mature)
QVQLKESGPELVKPGASVKISCKASGYTFTDYYINWVKQRPGQGLEWIGWIFPGSGSTYYNEKFKGKATLTVDKSSSTAYMLLSSLTSEDSAVYFCADYGSSYRYFDVWGAGTTVTVSS

SEQ ID NO: 190 - protein, 106 aa, Artificial Sequence
  Antibody F VL amino acid sequence (predicted mature)
DIVMTQSPAILSASPGEKVTMTCRASSSVSYMHWYQQKPGSSPKPWIYATSYLASGVPARFSGSGSGTSYSLTIGRVEAEDAATYYCQQWSSNPLTFGAGTKLELK

SEQ ID NO: 191 - protein, 8 aa, Artificial Sequence
  Antibody G HCDR1 amino acid sequence (IMGT definition)
GYTFTDYE

SEQ ID NO: 192 - protein, 8 aa, Artificial Sequence
  Antibody G HCDR2 amino acid sequence (IMGT definition)
IDPETGGT

SEQ ID NO: 193 - protein, 17 aa, Artificial Sequence
  Antibody G HCDR3 amino acid sequence (IMGT definition)
TRWGAITSFVALRGFAY

SEQ ID NO: 194 - protein, 12 aa, Artificial Sequence
  Antibody G LCDR1 amino acid sequence (IMGT definition)
QSLLNSGNQKNY

SEQ ID NO: 195 - protein, 3 aa, Artificial Sequence
  Antibody G LCDR2 amino acid sequence (IMGT definition)
GAS

SEQ ID NO: 196 - protein, 9 aa, Artificial Sequence
  Antibody G LCDR3 amino acid sequence (IMGT definition)
QNDHSYPPT

SEQ ID NO: 197 - protein, 124 aa, Artificial Sequence
  Antibody G VH amino acid sequence (predicted mature)
QVQLKQSGAELVRPGASVTLSCKASGYTFTDYEMHWVKQTPVHGLEWIGGIDPETGGTAYNQKFKGKAILTADKSSSTAYMELRSLTSEDSAVYYCTRWGAITSFVALRGFAYWGQGTLVTVSS

SEQ ID NO: 198 - protein, 113 aa, Artificial Sequence
  Antibody G VL amino acid sequence (predicted mature)
DIQMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPPTFGAGTKLELK

SEQ ID NO: 199 - protein, 8 aa, Artificial Sequence
  Antibody H HCDR1 amino acid sequence (IMGT definition)
GFTFSSYG

SEQ ID NO: 200 - protein, 8 aa, Artificial Sequence
  Antibody H HCDR2 amino acid sequence (IMGT definition)
ISRRSIYT

SEQ ID NO: 201 - protein, 17 aa, Artificial Sequence
  Antibody H HCDR3 amino acid sequence (IMGT definition)
ARLSLYDYDGARYTMDY

SEQ ID NO: 202 - protein, 6 aa, Artificial Sequence
  Antibody H LCDR1 amino acid sequence (IMGT definition)
QDVGTA

SEQ ID NO: 203 - protein, 3 aa, Artificial Sequence
  Antibody H LCDR2 amino acid sequence (IMGT definition)
WAS

SEQ ID NO: 204 - protein, 8 aa, Artificial Sequence
  Antibody H LCDR3 amino acid sequence (IMGT definition)
QQYSSYHT

SEQ ID NO: 205 - protein, 124 aa, Artificial Sequence
  Antibody H VH amino acid sequence (predicted mature)
EVKLVESGGDLVKPGGSLKLSCAASGFTFSSYGMSWVRQTPDKRLEWVATISRRSIYTYYPDSVQGRFTISRDNAKNTLYLQMSSLKSEDTAMYYCARLSLYDYDGARYTMDYWGQGTSVTVSS

SEQ ID NO: 206 - protein, 106 aa, Artificial Sequence
  Antibody H VL amino acid sequence (predicted mature)
DIKMTQSPKFMSTSVGDRVSITCKASQDVGTAVAWYQQKPGQSPKLLIYWASTRHTGVPDRFTGSGSGTDFTLTISNVQSEDLADYFCQQYSSYHTFGAGTKLEIK

SEQ ID NO: 207 - protein, 8 aa, Artificial Sequence
  Antibody J HCDR1 amino acid sequence (IMGT definition)
GYTFTSYW

SEQ ID NO: 208 - protein, 8 aa, Artificial Sequence
  Antibody J HCDR2 amino acid sequence (IMGT definition)
INPSNGGT

SEQ ID NO: 209 - protein, 17 aa, Artificial Sequence
  Antibody J HCDR3 amino acid sequence (IMGT definition)
ARGGIYYGNYDYYAMDY

SEQ ID NO: 210 - protein, 11 aa, Artificial Sequence
  Antibody J LCDR1 amino acid sequence (IMGT definition)
KSLLHSNGNTY

SEQ ID NO: 211 - protein, 3 aa, Artificial Sequence
  Antibody J LCDR2 amino acid sequence (IMGT definition)
RVS

SEQ ID NO: 212 - protein, 9 aa, Artificial Sequence
  Antibody J LCDR3 amino acid sequence (IMGT definition)
MQHLEYPFT

SEQ ID NO: 213 - protein, 124 aa, Artificial Sequence
  Antibody J VH amino acid sequence (predicted mature)
QVHLQQSGTELVKPGASVKLSCEASGYTFTSYWMHWVKQRPGQGLEWIGNINPSNGGTNYNEKFKSKATLTVDKSSSTAYMQLSSLTSEDSAVYYCARGGIYYGNYDYYAMDYWGQGTSVTVSS

SEQ ID NO: 214 - protein, 112 aa, Artificial Sequence
  Antibody J VL amino acid sequence (predicted mature)
DIVMTQAAPSVPVTPGESVSISCRSSKSLLHSNGNTYLYWFLQRPGQSPQLLIYRVSNLASGVPDRFSGSGSGTAFTLRISRVEAEDVGVYYCMQHLEYPFTFGGGTKLEIK

SEQ ID NO: 215 - protein, 8 aa, Artificial Sequence
  Antibody K HCDR1 amino acid sequence (IMGT definition)
GYAFTNYL

SEQ ID NO: 216 - protein, 8 aa, Artificial Sequence
  Antibody K HCDR2 amino acid sequence (IMGT definition)
INPGSGGT

SEQ ID NO: 217 - protein, 11 aa, Artificial Sequence
  Antibody K HCDR3 amino acid sequence (IMGT definition)
ARWGFYYAMDY

SEQ ID NO: 218 - protein, 6 aa, Artificial Sequence
  Antibody K LCDR1 amino acid sequence (IMGT definition)
QEISGY

SEQ ID NO: 219 - protein, 3 aa, Artificial Sequence
  Antibody K LCDR2 amino acid sequence (IMGT definition)
AAS

SEQ ID NO: 220 - protein, 9 aa, Artificial Sequence
  Antibody K LCDR3 amino acid sequence (IMGT definition)
LQYASYPLT

SEQ ID NO: 221 - protein, 118 aa, Artificial Sequence
  Antibody K VH amino acid sequence (predicted mature)
QVQLKQSGAELVRPGTSVKVSCKASGYAFTNYLIEWVKQRPGQGLEWIGVINPGSGGTNYNEKFKGKATLTADKSSSTAYMQLSSLTSEDSAVYFCARWGFYYAMDYWGQGTSVTVSS

SEQ ID NO: 222 - protein, 107 aa, Artificial Sequence
  Antibody K VL amino acid sequence (predicted mature)
DIVMTQSPSSLSASLGERVSLTCRASQEISGYLSWLQQKPDGTIKRLIYAASTLDSGVPKRFSGSRSGSDYSLTISSLESEDFADYYCLQYASYPLTFGAGTKLEIK

SEQ ID NO: 223 - protein, 14 aa, Homo sapiens
DAIWHFADNQLFVS

SEQ ID NO: 224 - protein, 10 aa, Homo sapiens
LFVSGPNGTA

SEQ ID NO: 225 - protein, 8 aa, Artificial Sequence
  HCDR2 Consensus
  MISC_FEATURE (1)..(1): Xaa=Val or Ile
  MISC_FEATURE (5)..(5): Xaa=Thr or Ser
XDPEXGGT

SEQ ID NO: 226 - protein, 15 aa, Homo sapiens
RDFGPRLFTALAGWG

SEQ ID NO: 227 - protein, 11 aa, Homo sapiens
RLFTALAGWGS

SEQ ID NO: 228 - protein, 14 aa, Homo sapiens
TALAGWGSAVFTTG

SEQ ID NO: 229 - protein, 17 aa, Artificial Sequence
  HCDR2 Consensus
  MISC_FEATURE (2)..(2): Xaa=Val or Ile
  MISC_FEATURE (6)..(6): Xaa=Thr or Ser
  MISC_FEATURE (10)..(10): Xaa=Gly or Ala
  MISC_FEATURE (16)..(16): Xaa=Arg or Lys
GXDPEXGGTXYNQKFXG

SEQ ID NO: 230 - protein, 6 aa, Artificial Sequence
  HCDR2 Consensus
  MISC_FEATURE (4)..(4): Xaa=Thr or Ser
DPEXGG

SEQ ID NO: 231 - protein, 8 aa, Artificial Sequence
  HCDR2 Consensus
  MISC_FEATURE (4)..(4): Xaa=Gly or Arg
ISRXSIYT

SEQ ID NO: 232 - protein, 17 aa, Artificial Sequence
  HCDR2 Consensus
  MISC_FEATURE (5)..(5): Xaa=Gly or Arg
  MISC_FEATURE (16)..(16): Xaa=Lys or Gln
TISRXSIYTYYPDSVXG

SEQ ID NO: 233 - protein, 6 aa, Artificial Sequence
  HCDR2 Consensus
  MISC_FEATURE (3)..(3): Xaa=Gly or Arg
SRXSIY

SEQ ID NO: 234 - protein, 8 aa, Artificial Sequence
  HCDR2 Consensus
  MISC_FEATURE (2)..(2): Xaa=Tyr or Phe
  MISC_FEATURE (7)..(7): Xaa=Asn or Ser
IXPGSGXT

SEQ ID NO: 235 - protein, 17 aa, Artificial Sequence
  HCDR2 Consensus
  MISC_FEATURE (1)..(1): Xaa=Glu or Trp
  MISC_FEATURE (3)..(3): Xaa=Tyr or Phe
  MISC_FEATURE (8)..(8): Xaa=Asn or Ser
  MISC_FEATURE (15)..(15): Xaa=Leu or Phe
XIXPGSGXTYYNEKXKG

(19) United States
(12) Patent Application Publication
(10) Pub. No.: US 2022/0298237 A1
(43) Pub. Date: Sep. 22, 2022

(54) ANTI AQP3 MONOCLONAL ANTIBODY SPECIFICALLY BINDING TO EXTRACELLULAR DOMAIN OF AQUAPORIN 3 (AQP3) AND USE THEREOF

(71) Applicant: Keio University, Tokyo (JP)

(72) Inventors: Mariko Chikuma, Shinjuku (JP); Masato Yasui, Kamakura (JP)

(73) Assignee: Keio University, Tokyo (JP)

(21) Appl. No.: 17/603,761

(22) PCT Filed: Apr. 17, 2020
(86) PCT No.: PCT/JP2020/016856

§ 371 Date: Oct. 14, 2021

(30) Foreign Application Priority Data
Apr. 17, 2019 (JP) PCT/JP2019/016429

(51) Int. Cl. C07K 16/28; A61P 29/00

(52) U.S. Cl. CPC C07K 16/28; A61P 29/00; C07K 2317/76; C07K 2317/51; C07K 2317/515; C07K 2317/92

(57) ABSTRACT

A subject of the present invention is to provide an anti AQP3 antibody specifically recognizing the extracellular domain of aquaporin 3 (AQP3), which is one type of a water channel protein. By selecting a monoclonal antibody which specifically binds to an oligopeptide included in loop C as the extracellular domains of AQP3, anti AQP3 antibodies that are desired in the present invention are provided. An anti AQP3 monoclonal antibody of the present invention can directly bind, from the outside of a cell, to AQP3 present in a cell membrane. Furthermore, as an anti AQP3 monoclonal antibody of the present invention can have an inhibitory activity, the function of permeating a low molecular weight molecule or the like, which is carried by AQP3, can be suppressed.

### FIELD OF INVENTION

The present invention relates to anti AQP 3 antibodies specifically binding to extracellular domain of aquaporin 3 (AQP3), and further relates to the use of the antibodies.

### BACKGROUND OF THE DISCLOSURE

A biological membrane has low permeability to water molecules as it is composed of a lipid bilayer. Due to this reason, when it is desired to transport (permeate) water molecules rapidly and also in a large amount across a biological membrane, a water channel comprised of a membrane protein is necessary. Aquaporin (AQP) as a water channel is a membrane protein which has fine holes (pores) which allow pass-through of water molecules only, and it was discovered from red blood cell membranes by Peter Agre's group in 1992. Since then, aquaporin has been discovered in various bacteria, animals, and plants, and is known to be a water channel that is commonly present in a biological system. It is also confirmed that a number of AQP molecular types (genes) are present even in one biological species. For example, 13 kinds of aquaporin molecular type, from AQP0 to AQP12, are confirmed in a human. In addition, functional differentiation among molecular types is recognized like molecular types allowing selective pass-through of water molecules (AQP1 and the like) and molecular types allowing pass-through of a low molecular weight material such as water molecule, glycerin, or hydrogen peroxide (AQP3 and the like). It is clearly shown that the 13 kinds of AQP molecular types exhibit various expression patterns in many organs, and, in an organ like a kidney in which water transport frequently occurs, expression of plural molecular types of aquaporin in one organ is recognized.

It has become gradually evident that an abnormal expression and/or function of aquaporin is related to certain disorders. For example, it is known that deficiency of AQP0 can result in congenital cataract. It is known that the reduced expression/function of AQP2 is related to diabetes insipidus, and, on the other hand, it is suggested that hyperactivity of AQP2 is related to edema, high blood pressure, and congestive heart failure, associated with pregnancy. In the case of neuromyelitis optica as a demyelinating disorder, it is known that anti AQP4 autoantibodies are involved with an occurrence of pathological conditions. It is also reported that there is a relation between a mutation in AQP5 and palmoplantar keratoderma (Verkman et al., Nat. Rev. Drug Discov. (2014) vol. 13, pp. 259-277).

Aquaporin is a membrane protein which traverses the cell membrane six times, and has six transmembrane domains and five loops connecting the transmembrane domains (loop A to loop E). Among the AQP polypeptides in AQP present in a cell membrane, each of the N-terminal regions, loop B, loop D, and C-terminal region is present at the cytoplasmic side, while each of loop A, loop C, and loop E is present at the extracellular side (FIG. 1). This six-transmembrane structure is commonly found in all AQP molecular types.

Although one molecule of aquaporin has one passage route, aquaporin is present as a multimer (homotetramer) in a biological membrane. In addition, aquaporin is re-sponsible for the function of passive transport of low molecular weight molecules like water molecules, glycerol, hydrogen peroxide, carbon dioxide, ammonia, and urea through a passage route.

Although various analyses have been made with regard to the expression characteristics or function of each molecular type of aquaporin, sufficient elucidation is yet to be made. As one reason of not having sufficient elucidation, non-availability of an anti-aquaporin antibody with a sufficient property of identifying each molecular type can be mentioned. At the present moment, there are several reports regarding the obtainment of an anti AQP antibody, and there is also an anti AQP antibody which is commercially supplied. However, most of those antibodies are polyclonal antibodies, and they have the intracellular domain of AQP as an epitope. With a polyclonal antibody, there are many cases in which the specific identifying property is not sufficient, and there is also limitation in that detection or measurement cannot be made with high precision. Furthermore, with a polyclonal antibody, it is practically impossible to carry out the isolation and purification of AQP-expressing cells. Because most of the anti AQP antibodies of a related art are an antibody which recognizes an epitope present inside a cell, there are also limitations when analyzing living cells.

Although the reason of having very limited example of obtaining an antibody which specifically recognizes the extracellular domain of aquaporin remains unclear, a membrane protein like aquaporin is difficult to be handled as an immunogen, and obtaining an antibody which specifically recognizes a membrane protein is not easy in general. It is also considered that, as the sequence conservation is relatively high among biospecies, it is difficult to produce a desired specific antibody when an animal of different species is immunized by using the aquaporin protein or a fragment thereof as an immunogen.

Like other molecular types of AQP, aquaporin 3 (AQP3) is a water channel protein which is localized in a biological membrane and formed of six transmembrane regions (transmembrane regions I to VI) each consisting of an a helix and five loops connecting them (loop A to loop E), and it has a structure in which both the N-terminal region and the C-terminal region are present at the cytoplasmic side. The a helix which traverses the biological membrane forms fine holes (pores) which allow pass-through of a water molecule or other low molecular weight components (glycerol and hydrogen peroxide).

It is known that AQP3 is expressed in various cells including epithelial cells, immune cells, and cancer cells. Keratinocytes are one of the cells in which AQP3 is expressed in a large amount. AQP3 is considered to play an important role in physiological moisturization of skin and recovery of skin wounds as it promotes transport of water and glycerol (JP 2011-32191). Meanwhile, for a skin disorder accompanying abnormal keratinocyte proliferation like psoriasis, actinic keratosis, ichthyosis, and seborrheic dermatitis, therapy based on suppression of AQP3 function by having, as a target, AQP3 as a factor for regulating cell proliferation of keratinocyte is suggested (WO 2014/013727). Involvement with skin tumorigenesis is also reported. A mechanism in which each AQP3 exhibits its physiological activity based on glycerol transporting activity for moisturization, oncogenesis, and recovery of barrier function in skin or based on water molecule transporting activity for recovery of wounded skin is suggested (Hara-Chikuma et al., J. Invest. Dermatol. (2008) vol. 128, pp. 2145-2151).

As for the relationship between AQP3 and cancer, many cases have been reported without being limited to skin cancer. Increased expression level of each AQP3 is confirmed in tissues of colorectal cancer, cervical cancer, liver cancer, lung cancer, esophageal cancer, kidney cancer, stomach cancer, tongue cancer, and the like. It is furthermore suggested that, in those cancers, the AQP3 function is related to progress level, prognosis, tumor angiogenesis, infiltration, metastasis of cancer, and energy metabolism of cancer tissues, and the like. Due to such reasons, although (lowering the expression level of) AQP3 has been suggested as a therapeutic target for those cancers, favorable results have not yet been obtained from an actual trial (Verkman et al., Nat. Rev. Drug Discov. (2014) vol. 13, pp. 259-277, Papadopoulos and Saadoun, Biochem. Biochim. Acta (2015) vol. 1848, pp. 2576-2583, and Wang et al., J. Transl. Med. (2015) vol. 13: 96).

The large intestine is known as one of other main tissues in which AQP3 is expressed, and there is a report indicating the relationship between the expression level and physiological state of AQP3 in intestinal epithelium. According to the report, it is evident that the expression level of AQP3 in large intestine is lowered by several laxatives. Severe constipation caused by morphine is associated with the increased expression level of AQP3 in large intestine (Ikarashi et al., Int. J. Mol. Sci. (2016) vol. 17, 1172).

For the analysis of AQP3, a compound suppressing the channel's activity of permeating water molecules or glycerol is reported as an AQP3 inhibitor (Zelenina et al., J. Biol. Chem. (2004) vol. 279, pp. 51939-51943 and Martins et al., PLoS ONE (2012) 7(5): e37435). Without being limited to the AQP3 inhibitor, most AQP inhibitors are metal compounds which contain a metal like mercury, copper, or gold. Being a metal compound means that there is a high possibility of exhibiting cytotoxicity. Due to such reasons, although certain usefulness is recognized for this AQP inhibitor, it is limited in terms of the application both in functional analysis using cultured cells and a test in which administration to a test animal is made. Furthermore, molecular type specificity for AQP of the AQP inhibitor as a metal compound is generally not high. For example, there is a report indicating a problem that it causes not only the inhibition on AQP3 but also functional inhibition of other AQP molecular types like AQP1 and AQP4. As such, the administration to a human as a clinical application of the AQP3 inhibitor is not pragmatically feasible.

As another approach of the AQP3 functional analysis, a case in which AQP3 deficient cells or AQP3 knock-down cells are used has been reported (Hara-Chikuma et al., Biochem. Biophys. Res. Commun. (2016) vol. 471, pp. 603-609). It is found that the cell proliferation property or cell migration is reduced and the response caused by inflammation (inflammatory response) is reduced in AQP3 deficient or knock-down cells. It is also reported that, when a treatment causing an inflammatory disorder like atopic dermatitis, psoriasis, asthma or the like is carried out for an AQP3 knock-out mouse, an occurrence of those inflammatory disorders is suppressed compared to a control in which a wild type mouse is used. It is also reported that, in a transplant experiment from cancer cells derived from human to a mouse, cancer malignancy can be suppressed according to knock-down of the expression or function of AQP3. For the knock-down, an example of using siRNA, shRNA, and miRNA is reported. However, all of those studies are just at a basic research stage, and development of a clinically applicable agent for regulating AQP3 expression is not achieved yet.

For having a progress in the analysis of AQP3, detecting at high precision the expression site or expression level of AQP3 is one of the necessary means. AQP3-specific detection is widely carried out based on detection of accumulation level of AQP3 mRNA by using a specific probe or primer. However, according to an analysis at nucleic acid level, it is impossible to know that AQP3 protein is actually present at which distribution and in which amount. Meanwhile, because an anti AQP3 antibody is established and several antibodies are commercially available, expression analysis of AQP3 can be also made. However, all of the commercially available anti AQP3 antibodies are a polyclonal antibody, and they are not specific enough for the high-precision analysis. Furthermore, because all of the commercially available anti AQP3 antibodies are an antibody which has, as an epitope, the intracellular domain present at N-terminal part or C-terminal part of the AQP3, it is difficult to have detection of AQP3 by an experiment using living cells or to be used for selecting and purifying AQP3-expressing cells using an antibody. Under the circumstances, a monoclonal antibody for AQP3, in particular, a monoclonal antibody specifically recognizing the extracellular domain of AQP3, is strongly desired.

### CITATION LIST

### Patent Literature

PTL 1: JP 2011-32191 A
PTL 2: WO 2014/013727 A1
PTL 3: WO 98/13388 A1
PTL 4: WO 91/09967 A
PTL 5: U.S. Pat. No. 5,530,101 A
PTL 6: U.S. Pat. No. 5,585,089 A
PTL 7: U.S. Pat. No. 5,693,761 A
PTL 8: U.S. Pat. No. 5,693,762 A
PTL 9: U.S. Pat. No. 6,180,370 B1
PTL 10: U.S. Pat. No. 5,225,539 A
PTL 11: EP 239400 A2
PTL 12: EP 592106 A1
PTL 13: EP 519596 A1
PTL 14: U.S. Pat. No. 5,565,332 A

### Non Patent Literature

NPL 1: Verkman et al., Nat. Rev. Drug Discov. (2014) vol. 13, pp. 259-277
NPL 2: Hara-Chikuma et al., J. Invest. Dermatol. (2008) vol. 128, pp. 2145-2151
NPL 3: Papadopoulos and Saadoun, Biochem. Biochim. Acta (2015) vol. 1848, pp. 2576-2583
NPL 4: Wang et al., J. Transl. Med. (2015) vol. 13: 96
NPL 5: Ikarashi et al., Int. J. Mol. Sci. (2016) vol. 17, 1172
NPL 6: Zelenina et al., J. Biol. Chem. (2004) vol. 279, pp. 51939-51943
NPL 7: Martins et al., PLoS ONE (2012) 7(5): e37435
NPL 8: Hara-Chikuma et al., Biochem. Biophys. Res. Commun. (2016) vol. 471, pp. 603-609
NPL 9: Riechmann et al., 1988, Nature 332:323-7
NPL 10: Padlan, 1991, Mol. Immunol., 28:489-498
NPL 11: Studnicka et al, 1994, Prot. Eng. 7:805-814
NPL 12: Roguska et al, 1994, Proc. Natl. Acad. Sci. 91:969-973

### SUMMARY OF INVENTION

### Technical Problem

An object of the present invention is to provide an anti AQP3 antibody specifically recognizing the extracellular domain of aquaporin 3 (AQP3), which is a kind of water channel protein.

### Solution to Problem

In order to provide an anti AQP3 antibody specifically recognizing the extracellular domain of AQP3, the inventors of the present invention performed intensive studies on the structure of AQP3, in particular, the structure of loop A, loop C, and loop E which constitute the extracellular domain, and found that, according to immunization of a host animal by using a fragment (oligopeptide) constituting a part of loop C (extracellular second loop) as an immunogen, sometimes together with AQP3-overexpressing cells, a desired antibody specifically recognizing AQP3 at an affinity of greater than or equal to 100 pM can be obtained, plural anti AQP3 monoclonal antibodies (anti AQP3 mAbs) derived from phage clones can be obtained from spleen and/or bone marrow of animals immunized with the peptide, the anti AQP3 mAb specifically binds to an AQP3 polypeptide and the aforementioned fragment, and the anti AQP3 mAb has an activity of specifically inhibiting the AQP3-based channel function, proliferation activity of AQP3-expressing cells, and/or migration activity of AQP3-expressing cells. Based on those findings, the inventors completed the present invention.

According to the present invention, an anti AQP3 antibody specifically recognizing the extracellular domain of AQP3 is provided. Furthermore, a composition containing an anti AQP3 antibody of the present invention, a reagent for detecting AQP3, a reagent for identifying and separating AQP3-expressing cells, and a reagent for measuring AQP3, which each contain an anti AQP3 antibody of the present invention, are provided. Furthermore, a kit including any of those reagents is provided. Furthermore, an anti AQP3 monoclonal antibody (inhibitory anti AQP3 mAb) which specifically binds to the extracellular domain of AQP3 and has an inhibitory activity for the channel function or the like of AQP3 is provided. Furthermore, a composition containing an inhibitory anti AQP3 mAb of the present invention, an AQP3 inhibitor containing an inhibitory anti AQP3 mAb of the present invention, and a pharmaceutical composition containing an inhibitory anti AQP3 mAb of the present invention are provided. Furthermore, an antibody drug conjugate (ADC) comprising an anti AQP3 antibody of the present invention and a cytotoxic agent, and pharmaceutical compositions comprising an ADC are provided. Furthermore, a method for detecting AQP3 by using an anti AQP3 antibody or reagent for detecting AQP3 of the present invention, a method for separating and purifying AQP3-expressing cells by using an anti AQP3 antibody or reagent for identifying and separating AQP3 of the present invention, and a method for measuring AQP3 by using an anti AQP3 antibody or reagent for detecting AQP3 of the present invention are provided. Furthermore, a method for inhibiting a function (channel function or the like) of AQP3 by using an inhibitory anti AQP3 mAb, composition containing an inhibitory anti AQP3 mAb, or AQP3 inhibitor of the present invention, and a method for inhibiting the transport of a low molecular weight material (water, glycerol, hydrogen peroxide, or the like) across a biological membrane by using an inhibitory anti AQP3 mAb, a composition containing the inhibitory anti AQP3 mAb, or AQP3 inhibitor of the present invention are provided. Still furthermore, a method for preventing/treating disorders associated with AQP3 by using an inhibitory anti AQP3 mAb, a composition containing the inhibitory anti AQP3 mAb, or pharmaceutical composition containing an inhibitory anti AQP3 mAb of the present invention is provided.

In one aspect, the present invention provides an anti AQP3 antibody or a functional fragment thereof that specifically binds with an affinity of greater than or equal to 100 pM to an oligopeptide whose amino acid sequence consists of ATYPSGHLDM (SEQ ID NO:1).

In another aspect, the present invention provides an anti AQP3 antibody or a functional fragment thereof comprising a heavy chain complementarity determining region 1 (HCDR1), a heavy chain complementarity determining region 2 (HCRD2), a heavy chain complementarity determining region 3 (HCDR3), a light chain complementarity determining region 1 (LCDR1), a light chain complementarity determining region 2 (LCDR2), and a light chain complementarity determining region 3 (LCDR3) comprising amino acid sequences selected from the sequences set forth in Table 6. CDR sequences are derived from the amino acid sequences using the sequences shown in Table 6 and as described in Example 17. The framework sequences for anti AQP3 antibodies or functional fragments thereof having CDR sequences described above can be murine framework sequences or human framework sequences.

In some embodiments, an antibody or functional fragment thereof can compete with another anti AQP3 antibody or functional fragment thereof of the present invention for binding to AQP3, e.g., human AQP3 expressed on the surface of HaCaT cells or mouse AQP3 expressed on the surface of PAM212 cells, or mouse macrophage cells. Assays that can be used to measure competition include ELISA and FACS assays.

In one example of a competition assay, cells expressing AQP3 on their surface (e.g., HaCaT cells) are adhered onto a solid surface, e.g., a microwell plate, by contacting the plate with a suspension of AQP3 expressing cells (e.g., over night at 4° C.). The plate is washed (e.g., 0.1% Tween 20 in PBS) and blocked (e.g., in Superblock, Thermo Scientific, Rockford, Ill.). A mixture of sub-saturating amount of a biotinylated first antibody (80 ng/mL) (the “reference” antibody) or competing anti AQP3 antibody (the “test” antibody) in serial dilution (e.g., at a concentration of 2.8 μg/mL, 8.3 μg/mL, or 25 μg/mL) in ELISA buffer (e.g., 1% BSA and 0.1% Tween 20 in PBS) is added to wells and plates are incubated for 1 hour with gentle shaking. The reference antibody can be an antibody of the invention. The plate is washed, 1 μg/mL HRP-conjugated Streptavidin diluted in ELISA buffer is added to each well and the plates incubated for 1 hour. Plates are washed and bound antibodies are detected by addition of substrate (e.g., TMB, Biofx Laboratories Inc., Owings Mills, Md.). The reaction is terminated by addition of stop buffer (e.g., Bio FX Stop Reagents, Biofx Laboratories Inc., Owings Mills, Md.) and the absorbance is measured at 650 nm using microplate reader (e.g., VERSAmax, Molecular Devices, Sunnyvale, Calif.). Variations on this competition assay can also be used to test competition between a first anti AQP3 antibody of the present invention and a second AQP3 antibody of the present invention. Other formats for competition assays are known in the art and can be employed.

In various embodiments of the above-described competition assay, a test anti AQP3 antibody of the present invention that competes with a reference AQP3 antibody of the present invention reduces the binding of the reference anti AQP3 antibody by at least 30%, by at least 40%, by at least 50%, by at least 60%, by at least 70%, by at least 80%, by at least 90%, by at least 95%, by at least 99% or by a percentage ranging between any of the foregoing values (e.g., a test anti AQP3 antibody of the present invention reduces the binding of a labeled reference anti AQP3 antibody of the present invention by 50% to 70%) when the test anti-AQP3 antibody is used at a concentration of 0.08 μg/mL, 0.4 μg/mL, 2 μg/mL, 10 μg/mL, 50 μg/mL, 100 μg/mL or at a concentration ranging between any of the foregoing values (e.g., at a concentration ranging from 2 μg/mL to 10 μg/mL).

In various embodiments of the above-described competition assay, a test anti AQP3 antibody of the present invention that competes with a reference AQP3 antibody of the present invention reduces the binding of the reference anti AQP3 antibody by at least 30%, by at least 40%, by at least 50%, by at least 60%, by at least 70%, by at least 80%, by at least 90%, by at least 95%, by at least 99% or by a percentage ranging between any of the foregoing values (e.g., a test anti AQP3 antibody of the present invention reduces the binding of a labeled reference anti AQP3 antibody of the present invention by 50% to 70%) when the test anti-AQP3 antibody is used at a concentration of 2 pM, 10 pM, 50 pM, 100 pM or at a concentration ranging between any of the foregoing values (e.g., at a concentration ranging from 2 pM to 10 pM).

In other embodiments of the above-described competition assay, a test anti AQP3 antibody of the present invention reduces the binding of a labeled reference anti AQP3 antibody by at least 40%, by at least 50%, by at least 60%, by at least 70%, by at least 80%, by at least 90%, or by a percentage ranging between any of the foregoing values (e.g., a test anti AQP3 antibody of the present invention reduces the binding of a labeled reference anti AQP3 antibody of the present invention by 50% to 70%) when the test anti AQP3 antibody is used at a concentration of 0.4 μg/mL, 2 μg/mL, 10 μg/mL, 50 μg/mL, 250 μg/mL or at a concentration ranging between any of the foregoing values (e.g., at a concentration ranging from 2 μg/mL to 10 μg/mL).

According to certain embodiments, the present invention includes an anti-AQP3 antibody or a functional fragment thereof comprising: a) a heavy chain complementarity determining region 1 (HCDR1) comprising the amino acid sequence X1FSLX2X3YA (SEQ ID NO:3), where X1 is G or R, X2 is S, Y, or N, and X3 is S, G, N, or T; b) a heavy chain complementarity determining region 2 (HCRD2) comprising the amino acid sequence INNDX4X5X6ST (SEQ ID NO:4), where X4 is G, I, or V,X5 is R, V, I, or S, and X6 is S or G; c) a heavy chain complementarity determining region 3 (HCDR3) comprising the amino acid sequence ARGGTSGYDI (SEQ ID NO:5); d) a light chain complementarity determining region 1 (LCDR1) comprising the amino acid sequence X7SVYKNY (SEQ ID NO:6), where X7 is P or Q; e) a light chain complementarity determining region 2 (LCDR2) comprising the amino acid sequence X8AS (SEQ ID NO:7), where X8 is G or K; and f) a light chain complementarity determining region 3 (LCDR3) comprising the amino acid sequence AGGYX9GX10X11DIFX12 (SEQ ID NO:8), where X9 is R or I, X10 is S or Y, X11 is S, G, or R, and X12 is A or S, in particular when X1 is G, X1 is R, X2 is S, X2 is Y, X2 is N, X3 is S, X3 is G, X3 is N, X3 is T, X4 is G, X4 is I, X4 is V, X5 is R, X5 is V, X5 is I, X5 is S, X6 is S, X6 is G, X7 is P, X7 is Q, X8 is G, X8 is K, X9 is R, X9 is I, X10 is S, X10 is Y, X11 is S, X11 is G, X11 is R, X12 is A, X12 is S.

According to further embodiments, the present invention includes an anti-AQP3 antibody or a functional fragment thereof comprising: a) a heavy chain complementarity determining region 1 (HCDR1) comprising the amino acid sequence X13FSLX14X15YA (SEQ ID NO:9), where X13 is G or R, X14 is S, Y, or N, and X15 is S, N, or T; b) a heavy chain complementarity determining region 2 (HCRD2) comprising the amino acid sequence INNDX16ISST (SEQ ID NO:10), where X16 is G or V; c) a heavy chain complementarity determining region 3 (HCDR3) comprising the amino acid sequence ARGGTSGYDI (SEQ ID NO:5); d) a light chain complementarity determining region 1 (LCDR1) comprising the amino acid sequence PSVYKNY (SEQ ID NO:11); e) a light chain complementarity determining region 2 (LCDR2) comprising the amino acid sequence GAS (SEQ ID NO:12); and f) and a light chain complementarity determining region 3 (LCDR3) comprising the amino acid sequence AGGYX17GSX18DIFX19 (SEQ ID NO:13), where X17 is R or I X18 is S or R, and X19 is A or S, in particular when X13 is G, X13 is R, X14 is S, X14 is Y, X14 is N, X15 is S, X15 is N, X15 is T, X16 is G, X16 is V, X17 is R, X17 is I, X18 is S, X18 is R, X19 is A, or X19 is S.

According to still further embodiments, the present invention includes an anti-AQP3 antibody or a functional fragment as described above comprising the HCDR1, HCDR2, HCDR3, LCDR1, LCDR2, and LCDR3 sequences of one of the binders set forth in Table 7, in particular when the anti-AQP3 antibody or a functional fragment thereof comprises the HCDR1, HCDR2, HCDR3, LCDR1, LCDR2, and LCDR3 sequences of BC—B10 as set forth in Table 7; the HCDR1, HCDR2, HCDR3, LCDR1, LCDR2, and LCDR3 sequences of BC—H9 as set forth in Table 7; the HCDR1, HCDR2, HCDR3, LCDR1, LCDR2, and LCDR3 sequences of SC—B6 as set forth in Table 7; or the HCDR1, HCDR2, HCDR3, LCDR1, LCDR2, and LCDR3 sequences of SC—F8 as set forth in Table 7.

According to even further embodiments, the present invention includes an anti-AQP3 antibody or a functional fragment as described above comprising variable heavy (VH) and variable light (VL) chain sequences of one of the binders set forth in Table 8, in particular when the VH and VL comprise the VH and VL sequences of BC—B10; the VH and VL sequences of BC—H9; the VH and VL sequences of SC—B6; or the VH and VL sequences of SC—F8.

According to certain embodiments, the present invention includes an anti AQP3 antibody or a functional fragment thereof that specifically binds to an oligopeptide whose amino acid sequence comprises or consists of ATYPSGHLDM (SEQ ID NO:1), in particular when the anti AQP3 antibody or a functional fragment thereof specifically binds to human and/or mouse AQP3, and further when the anti AQP3 antibody or a functional fragment thereof specifically binds the extracellular portion of human and/or mouse AQP3, especially when AQP3 binds to the extracellular portion of cell surface expressed human and/or mouse AQP3, in particular when the cells are HaCaT cells or PAM212 cells. According to further embodiments, the present invention includes an anti AQP3 antibody or a functional fragment thereof that specifically binds to an oligopeptide whose amino acid sequence comprises or consists of ATYPSGHLDM (SEQ ID NO:1) and binds with an affinity of greater than 100 pM, in particular when the anti AQP3 antibody or a functional fragment thereof specifically binds to Loop C, or when the antibody or functional fragment thereof binds to human and/or mouse AQP3.

According to certain embodiments, the present invention includes an anti AQP3 antibody or a functional fragment thereof that competes with the antibody or functional fragment thereof for binding to an oligopeptide whose amino acid sequence comprises or consists of SEQ ID NO:1. According to further embodiments, the present invention includes an anti AQP3 antibody or a functional fragment thereof that competes with the antibody or functional fragment thereof for binding to loop C of human or mouse AQP3. According to still further embodiments, the present invention includes an anti AQP3 antibody or a functional fragment thereof that competes with the antibody or functional fragment thereof for binding to human or mouse AQP3, especially when AQP3 is cell surface expressed, more specifically on HaCaT cells or PAM212 cells.

According to certain embodiments, the present invention includes an anti AQP3 antibody or a functional fragment thereof has an inhibitory activity on at least one function of human and/or mouse AQP3, specifically when the inhibitory activity of at least one function of human and/or mouse AQP3 is reduction in H₂O₂ transport, in particular when the inhibitory function is at least 50% reduction in H₂O₂ transport, specifically when the reduction in H₂O₂ transport is measured according to the assay described in Example 14.

According to certain embodiments, the present invention includes an anti AQP3 antibody or a functional fragment thereof that specifically binds to ATYPSGHLDM (SEQ ID NO:1), when the antibody or functional fragment thereof inhibits a functional response of keratinoid or immune cells (e.g., macrophages and T-cells) that are dependent on transport of H₂O₂ in particular when the functional response is inhibited by at least 50% compared to a non-AQP3 antibody, in particular when the reduction in H₂O₂ transport is measured according to the assay described in Example 14.

According to certain embodiments, the present invention includes an anti AQP3 antibody or a functional fragment thereof that specifically binds to Loop C of human AQP3, when the antibody or functional fragment thereof inhibits functional responses of immune cells that are dependent on transport of H₂O₂, and when that reduction is by at least 50%, as measured according to the assay described in Example 14.

According to certain embodiments, the present invention includes methods for producing an anti AQP3 antibody comprising steps of a) injecting an animal with SEQ ID NO:1; b) collecting one or more organs from the animal containing cells that produce antibodies; c) isolating mRNA from the organs; d) creating an antibody phage library using the mRNA; and e) screening the antibody phage library created in step d) to identify one or more antibodies that bind to SEQ ID NO:1, in particular when the organs are selected from spleen and bone marrow. According to further embodiments, the present invention includes methods for inhibiting at least one function of AQP3 comprising a step of contacting an AQP3 containing sample with an anti AQP3 antibody or a functional fragment thereof that specifically binds to SEQ ID NO:1. According to still further embodiments, the present invention includes methods for inhibiting at least one function of AQP3 comprising a step of contacting an AQP3 containing sample with an anti AQP3 antibody or a functional fragment thereof that specifically binds to Loop C of human AQP3. According to even further embodiments, the present invention includes methods for inhibiting at least one function of AQP3 comprising a step of contacting an AQP3 containing sample with an anti AQP3 antibody or a functional fragment thereof that specifically binds to the extracellular portion of human AQP3.

According to certain embodiments, the present invention includes methods for inhibiting transport of H₂O₂ across a membrane comprising a step of contacting a sample having a membrane including AQP3 with an anti AQP3 antibody or a functional fragment thereof that specifically binds to SEQ ID NO:1. According to further embodiments, the present invention includes methods for inhibiting transport of H₂O₂ across a membrane comprising a step of contacting a sample having a membrane including AQP3 with an anti AQP3 antibody or a functional fragment thereof that specifically binds to the extracellular portion of human AQP3. According to still further embodiments, the present invention includes methods for inhibiting transport of H₂O₂ across a membrane comprising a step of contacting a sample having a membrane including AQP3 with an anti AQP3 antibody or a functional fragment thereof that specifically binds to Loop C of human AQP3.

According to certain embodiments, the present invention includes methods for separating and/or purifying AQP3-expressing cells comprising a step of contacting a sample including cells with an anti AQP3 antibody or a functional fragment thereof that specifically binds to SEQ ID NO:1. According to further embodiments, the present invention includes methods for separating and/or purifying AQP3-expressing cells comprising a step of contacting a sample including cells with an anti AQP3 antibody or a functional fragment thereof that specifically binds to Loop C of human AQP3. According to still further embodiments, the present invention includes methods for separating and/or purifying AQP3-expressing cells comprising a step of contacting a sample including cells with an anti AQP3 antibody or a functional fragment thereof that specifically binds to the extracellular portion of human AQP3.

According to certain embodiments, the present invention includes methods for measuring AQP3 comprising a step of contacting a sample with an anti AQP3 antibody or a functional fragment thereof that specifically binds to SEQ ID NO:1. According to further embodiments, the present invention includes methods for measuring AQP3 comprising a step of contacting a sample with an anti AQP3 antibody or a functional fragment thereof that specifically binds to the Loop C of human AQP3. According to still further embodiments, the present invention includes methods for measuring AQP3 comprising a step of contacting a sample with an anti AQP3 antibody or a functional fragment thereof that specifically binds to the extracellular portion of human AQP3.

In some aspects, the present invention relates to the following (1) to (69).

(1) An anti AQP3 antibody specifically recognizing the extracellular domain of aquaporin 3 (AQP3) or a functional fragment thereof.

(2) The antibody or functional fragment thereof described in above 1, in which the extracellular domain is loop C.

(3) The antibody or functional fragment thereof described in above (1) or (2) specifically binding to an oligopeptide composed of ten amino acid residues at the C-terminal side of loop C that are adjacent to the boundary to the transmembrane region IV.

(4) The antibody or functional fragment thereof described in above (3), in which the amino acid sequence of the oligopeptide composed of ten amino acid residues at the C-terminal side of loop C, that are adjacent to the boundary to the transmembrane region IV, is ATYPSGHLDM (SEQ ID NO: 1).

(5) The antibody or functional fragment thereof described in any one of above (1) to (4), which is a mouse antibody, a rat antibody, a rabbit antibody, a guinea pig antibody, a sheep antibody, a goat antibody, a donkey antibody, a chicken antibody, or a camel antibody.

(6) The antibody or functional fragment thereof described in any one of above (1) to (5), which is a mouse antibody.

(7) The antibody or functional fragment thereof described in any one of above (1) to (6), which is labeled with a reporter material.

(8) The antibody or functional fragment thereof described in above (7), in which the reporter material is selected from the group consisting of a radioactive isotope, a metal micro particle, an enzyme, a fluorescent material, and a luminescent material.

(9) The antibody or a functional fragment thereof described in any one of above (1) to (8), which is immobilized on a solid support.

(10) The antibody or functional fragment thereof described in above (9), in which the solid support is selected from the group consisting of a micro plate, a glass plate, a plastic plate, a syringe, a vial, a column, a magnetic particle, a micro bead made of resin, a porous membrane, a porous carrier, and a microchip.

(11) The antibody or functional fragment thereof described in any one of above (1) to (10) specifically binding to AQP3 derived from a human and/or a mouse.

(12) The antibody or functional fragment thereof described in any one of above (1) to (11), which specifically binds to AQP3 derived from human.

(13) The antibody or functional fragment thereof described in any one of above (1) to (12), in which the antibody is an immunoglobulin molecule of IgG or IgM.

(14) The antibody or functional fragment thereof described in any one of above (1) to (13), in which the antibody is an immunoglobulin molecule of IgG.

(15) The antibody or functional fragment thereof described in any one of above (1) to (14) having an inhibitory activity on function of AQP3.

(16) The antibody or a functional fragment thereof described in above (15), in which the function of AQP3 is at least one activity selected from the group consisting of an activity of transporting (permeating) a low molecular weight material by AQP3, an activity of promoting cell proliferation of AQP3-expressing cells, an activity of promoting cell migration of AQP3-expressing cells, and an activity of inducing an inflammatory response and a disorder response associated with AQP3.

(17) The antibody or functional fragment thereof described in any one of above (1) to (16), in which the antibody is a monoclonal antibody.

(18) The antibody or functional fragment thereof described in above (17), in which heavy chain CDR1, CDR2, and CDR3 are composed of the amino acid sequence represented by SEQ ID NO: 3, SEQ ID NO: 4, and SEQ ID NO: 5, respectively, and light chain CDR1, CDR2, and CDR3 are composed of the amino acid sequence represented by SEQ ID NO: 6, SEQ ID NO: 7, and SEQ ID NO: 8, respectively.

(19) The antibody or functional fragment thereof described in above (17) or (18), in which the heavy chain variable region is composed of the amino acid sequence represented by any of the sequences for heavy chain variable region shown in Table 8 and the light chain variable region is composed of the amino acid sequence represented by any of the sequences for light chain variable region shown in Table 8.

(20) The antibody or functional fragment thereof described in above (17), in which heavy chain CDR1, CDR2, and CDR3 are composed of the amino acid sequence represented by SEQ ID NO: 9, SEQ ID NO: 10, and SEQ ID NO:5, respectively, and light chain CDR1, CDR2, and CDR3 are composed of the amino acid sequence represented by SEQ ID NO: 11, SEQ ID NO: 12, and SEQ ID NO: 13, respectively.

(21) The antibody or functional fragment thereof described in above (17) or (20), in which the heavy chain variable region is composed of the amino acid sequence represented by any of the sequences for heavy chain variable region shown in Table 8 and the light chain variable region is composed of the amino acid sequence represented by any of the sequences for light chain variable region shown in Table 8.

(22) The monoclonal antibody described in any one of above (1), (7), (18), (20) and (21) in which the antibody is a chimeric antibody or a humanized antibody having a constant region of a human antibody.

(23) A composition comprising the antibody or fragment thereof described in any one of above (1) to (22).

(24) The composition described in above (23), which is a reagent for detecting AQP3.

(25) The composition described in above (23), which is a reagent for identifying, separating, or purifying AQP3-expressing cells.

(26) The composition described in above (24) or (25), which is a reagent for measuring an expression amount of AQP3.

(27) A kit comprising the composition described in any one of above (23) to (26).

(28) A composition comprising the monoclonal antibody or fragment thereof described in any one of above (17) to (21), in which the monoclonal antibody or a functional fragment thereof has an inhibitory activity on function of AQP3.

(29) The composition described in above (28), in which the function of AQP3 is at least one activity selected from the group consisting of an activity of transporting a low molecular weight material by AQP3, an activity of promoting cell proliferation of AQP3-expressing cells, and an activity of promoting cell migration of AQP3-expressing cells.

(30) The composition described in above (28) or (29) which is a pharmaceutical composition further including a pharmaceutically acceptable carrier.

(31) The composition described in above (29) or (30) for use in treating cancer.

(32) The composition described in above (31), in which the cancer is cancer selected from the group consisting of colorectal cancer, cervical cancer, liver cancer, lung cancer, esophageal cancer, kidney cancer, stomach cancer, tongue cancer, skin cancer, and breast cancer.

(33) The composition described in above (31) or (32), in which the treatment is selected from the group consisting of suppression of a progress (proliferation) of cancer, suppression of tumor angiogenesis, suppression of infiltration, suppression of metastasis, suppression of energy metabolism in cancer tissues, and improvement of prognosis of a patient.

(34) The composition described in above (28) or (29), for use in preventing and/or treating a skin disorder.

(35) The composition described in above (34), in which the skin disorder is selected from the group consisting of psoriasis, actinic keratosis, ichthyosis, and seborrheic dermatitis.

(36) The composition described in above (29) or (30) for use in preventing and/or treating an inflammatory disorder.

(37) The composition described in above (36), in which the inflammatory disorder is selected from the group consisting of atopic dermatitis, psoriasis, asthma, chronic obstructive pulmonary disease, and hepatitis (e.g., acute hepatitis or acute hepatic disorder).

(38) The composition described in above (29) or (30), for use in treating an abnormality in bowel movement.

(39) The composition described in above (38), in which the abnormality in bowel movement is constipation.

(40) A method for detecting AQP3 comprising a step of contacting a sample with the antibody or fragment thereof described in any one of above (1) to (22), or with the composition described in above (23) or (24).

(41) The method described in above (40), in which it is carried out by using the kit described in above (27).

(42) The method described in above (40) or (41), in which the sample contains a cell, a living body tissue, an organ, or an individual subject.

(43) The method described in above (42), in which the sample contains a cell, a living body tissue, or an organ, and which is carried out in vitro.

(44) The method described in above (42), which is carried out in vivo (optionally with the proviso that a case of having an individual human or an individual animal as a sample is excluded).

(45) A method for separating and/or purifying AQP3-expressing cells from a sample comprising AQP3-expressing cells, the method comprising a step of contacting the sample with the antibody or a functional fragment thereof described in any one of above (1) to (22), or with the composition described in above (23) or (25).

(46) The method described in above (45), which is carried out by using the kit described in above (27).

(47) The method described in above (45) or (46), in which the sample is a sample containing living cells.

(48) A method for measuring AQP3 comprising a step of contacting a sample with the antibody or a functional fragment thereof described in any one of above (1) to (22), or with the composition described in above (23), (24), or (26).

(49) The method described in above (44), which is carried out by using the kit described in above (27).

(50) The method described in above (48) or (49), in which the sample contains a cell or a cell extract.

(51) A method for inhibiting at least one function of AQP3 comprising a step of contacting a sample including AQP3 with the antibody or a functional fragment thereof described in any one of above (1) to (22), or with the composition described in above (23).

(52) The method described in above (51), in which the sample containing AQP3 is a reconstituted membrane containing recombinant AQP3, or a cell group, living body tissues, an organ, or an individual containing AQP3-expressing cells.

(53) The method described in above (51) or (52), in which the contacting step is a step of contacting the sample with the monoclonal antibody or a functional fragment thereof described in any one of above (17) to (22) or with a composition containing the monoclonal antibody described in any one of above (17) to (22).

(54) The method described in above (53), in which the monoclonal antibody described in any one of above (17) to (22) or a functional fragment thereof has an activity of inhibiting at least one function of AQP3.

(55) The method described in above (54), in which the function of AQP3 is at least one activity selected from the group consisting of an activity of transporting a low molecular weight material by AQP3, an activity of promoting cell proliferation of AQP3-expressing cells, an activity of promoting cell migration of AQP3-expressing cells, and an activity of inducing an inflammatory response and a disorder response associated with AQP3.

(56) A method for inhibiting transport of a low molecular weight material across a membrane comprising a step of contacting a sample having a membrane including AQP3 with the antibody or a functional fragment thereof described in any one of above (1) to (22) or with the composition described in above (23).

(57) The method described in above (56), in which the membrane containing AQP3 is a reconstituted membrane containing recombinant AQP3 or a biological membrane of AQP3-expressing cells.

(58) The method described in above (56) or (57), in which the contacting step is a step of contacting with the monoclonal antibody or a functional fragment thereof described in any one of above (17) to (22) or with a composition containing the monoclonal antibody described in any one of above (17) to (22).

(59) The method described in above (58), in which the monoclonal antibody described in any one of above (17) to (22) or a functional fragment thereof has an activity of inhibiting a function of AQP3.

(60) The method described in above (59), in which the function of AQP3 is an activity of transporting a low molecular weight material by AQP3.

(61) The method described in any one of above (56) to (60), in which the low molecular weight material is selected from the group consisting of water molecule, glycerol, and hydrogen peroxide.

(62) A method for prevention and/or treatment of a disorder associated with AQP3 including a step of administering the composition described in any one of above (28) to (37) to a subject who is in need of treatment.

(63) The method described in above (62), in which the disorder associated with AQP3 is associated with an increased expression level of AQP3.

(64) The method described in above (63), in which the disorder associated with AQP3 is selected from the group consisting of cancer, a skin disorder, and an inflammatory disorder.

(65) A method of ameliorating an abnormality in bowel movement including a step of administering the composition described in above (28) to (30), (38), or (39) to a subject with an abnormality in bowel movement in which the abnormality in bowel movement is constipation.

(66) The composition described in above (29) or (30), which is for use in a method of treating a disorder associated with AQP3.

(67) The monoclonal antibody described in any one of above (17) to (22) or a functional fragment thereof, which is for use in a method of treating a disorder associated with AQP3.

(68) Use of the composition described in above (29) or (30) for producing a pharmaceutical composition for preventing and/or treating a disorder associated with AQP3.

(69) Use of the monoclonal antibody or a functional fragment thereof described in any one of above (17) to (22) for producing a pharmaceutical composition for preventing and/or treating a disorder associated with AQP3.

### Advantageous Effects of Invention

With an anti AQP3 antibody or a functional fragment thereof of the present invention which specifically recognizes the extracellular domain of AQP3, detection of AQP3-expressing cells or measurement of AQP3 expression level can be carried out. Furthermore, because an anti AQP3 antibody or a functional fragment thereof of the present invention can specifically bind to AQP3 present in cell membrane of living cells, staining of tissues or an organ containing AQP3-expressing cells or separation and purification of AQP3-expressing cells can be carried out. Furthermore, because in some embodiments an anti AQP3 antibody or a functional fragment thereof of the present invention can not only recognize specifically a peptide included in loop C of AQP3 but can also specifically bind to AQP3, it can inhibit one or more functions of AQP3. By inhibiting one or more functions of AQP3, it is possible to prevent and/or treat a disorder associated with AQP3 which is associated with an increase in AQP3 expression level. In a case in which the disorder associated with AQP3 is cancer, it is possible to have suppression of a progress (proliferation) of cancer, suppression of tumor angiogenesis, suppression of infiltration, suppression of metastasis, suppression of energy metabolism in cancer tissues, improvement of prognosis of a cancer patient, or a combination of the foregoing. It is also possible to alleviate an abnormality in bowel movement which is associated with an increase in AQP3 expression level.

### BRIEF DESCRIPTION OF DRAWINGS

FIG. 1 is a diagram illustrating the molecular structure of aquaporin. It has a transmembrane structure of traversing, from the N-terminal to the C-terminal, the membrane six times and, in the five regions connected between the six transmembrane domains of transmembrane domains I to VI, five loops (loop A to loop E) are included. Among those loops, loop A, loop C, and loop E are present at the extracellular side while loop B and loop D are present at the intracellular side, respectively. The N-terminal region and the C-terminal region are all included in the intracellular domain. Two NPAs shown in the drawing indicate an NPA box consisting of three amino acid residues of asparagines-proline-alanine. The NPA box is present inside an aquaporin molecule and it is known to be widely preserved among biospecies.

FIG. 2 is a diagram showing the result of testing the binding property of anti AQP3 antibodies to a peptide having the amino acid sequence of SEQ ID NO:1. Left panel shows the results for antibodies C, E, H, J, and a negative control IgG antibody (IgG). Right panel shows the results for antibodies B, G, K, A, D, and F.

FIG. 3 is a diagram showing the result of testing the binding property of anti AQP3 antibodies to cell lysate of AQP3 overexpressing HEK293T cells (AQP3). Cell lysate from HEK293T cells not overexpressing AQP3 was used as control (N.C.).

FIG. 4 is a diagram showing the result of testing the binding property of an anti AQP3 antibody (antibody J).

FIG. 5A is a diagram showing the result of testing the binding property of an anti AQP3 antibody (antibody J) to mouse epithelial cells (PAM212 cells).

FIG. 5B is a diagram showing the result of testing the binding property of antibodies A, B, C, D, E, F, G, H, and J to mouse epithelial cells (PAM212 cells).

FIG. 5C is a diagram showing the result of testing the binding property of antibodies A, B, C, D, E, F, G, H, and J to human epithelial cells (HaCaT cells).

FIG. 6A is a diagram showing the result of testing the binding property of antibody G to human epithelial cells (HaCaT cells).

FIG. 6B is a diagram showing the result of testing the binding property of antibody H to human epithelial cells (HaCaT cells).

FIG. 6C is a diagram showing the result of testing the binding property of antibody J to human epithelial cells (HaCaT cells).

FIG. 6D is a diagram showing the result of testing the binding property of antibody E to HEK293 cells overexpressing mouse AQP3.

FIG. 6E is a diagram showing the result of testing the binding property of antibody H to HEK293 cells overexpressing mouse AQP3.

FIG. 6F is a diagram showing the result of testing the binding property of antibody J to HEK293 cells overexpressing mouse AQP3.

FIG. 6G is a diagram showing the result of testing the binding properties of antibody E to HEK293 cells overexpressing mouse AQP3.

FIG. 6H is a diagram showing the result of testing the binding properties of antibody E to HEK293 cells overexpressing human AQP3.

FIG. 7A is a diagram showing the result of carrying out immunostaining for AQP3-expressing cells (mouse macrophages) by using anti AQP3 antibodies H and J.

FIG. 7B is a diagram showing the result of carrying out immunostaining for AQP3-expressing cells (mouse macrophages)(top panel) and AQP3 knock-out cells by using an anti AQP3 antibody (antibody J).

FIG. 8A is a diagram showing the result of testing the activity on cell proliferation of an anti AQP3 antibody (antibody G or antibody J) using mouse epithelial cells (PAM212 cells).

FIG. 8B is a diagram showing the result of testing the activity on cell proliferation of an anti AQP3 antibody (antibody J) by using mouse epithelial cells (PAM212 cells).

FIG. 8C is a diagram showing the result of testing the activity on cell proliferation of an anti AQP3 antibody (antibody A, B, C, D, E, F, G, H, or J).

FIG. 9 is a diagram showing the result of testing the activity on cell proliferation of an anti AQP3 antibody (antibody G, H, or J) using human epithelial cells (HaCaT cells).

FIG. 10 is a diagram showing the result of testing the activity on cell proliferation of anti AQP3 antibodies (antibodies G, H, and J) by using human epithelioid carcinoma cells (A431 cells).

FIG. 11 is a diagram showing the result of testing the functional inhibition effect of an anti AQP3 antibody (antibody J) on the hydrogen peroxide permeation function in mouse macrophage cells as AQP3-expressing cells.

FIG. 12 is a diagram showing the result of testing the functional inhibition effect of anti AQP3 antibodies (antibodies A, B, C, D, E, F, G, H, and J) on the hydrogen peroxide permeation function in mouse macrophage cells as AQP3-expressing cells.

FIG. 13 is a diagram showing the result of testing the functional inhibition effect of an anti AQP3 antibody (antibody J) on the LPS responsive p65 activation (p65 phosphorylation) in mouse macrophage cells as AQP3-expressing cells.

FIG. 14A is a diagram showing the result of testing the inhibitory effect of an anti AQP3 antibody (antibody J) on acute liver disorder (inflammatory response and disorder response) which was caused in a mouse by treatment with carbon tetrachloride. The test was carried out by having the AST level in blood serum as an indicator.

FIG. 14B is a diagram showing the result of testing the inhibitory effect of an anti AQP3 antibody (antibody J) on acute liver disorder (inflammatory response and disorder response) which was caused in a mouse by treatment with carbon tetrachloride. The test was carried out by having the ALT level in blood serum as an indicator.

FIG. 15A is a diagram showing the result of testing the inhibitory effect of an anti AQP3 antibody (antibody J) on acute liver disorder (inflammatory response and disorder response) which was caused in a mouse by treatment with carbon tetrachloride. The test was carried out by having the TNF-α mRNA expression level in a RNA sample, which was derived from liver, as an indicator.

FIG. 15B is a diagram showing the result of testing the inhibitory effect of an anti AQP3 antibody (antibody J) on acute liver disorder (inflammatory response and disorder response) which was caused in a mouse by treatment with carbon tetrachloride. The test was carried out by having the IL-6 mRNA expression level in a RNA sample, which was derived from liver, as an indicator.

FIG. 16A is a graph showing the result of an ELISA analysis testing the binding property of anti AQP3 antibodies SC—F8 (circles), BC—H9 (gray squares), BC—B10 (triangles), and SC—B6 (exes)) compared to anti AQP3 antibodies (antibody C (diamonds) and antibody J (black squares)) to a peptide having the amino acid sequence of SEQ ID NO:1. Also shown is a dashed line indicating the 50% binding response for SC—F8, BC—H9, BC—B10, and SC—B6. The amount of antibody needed for the 50% binding response is 0.01 μg/mL for SC—F8, BC—H9, BC—B10, and SC—B6 compared to approximately 0.1 μg/mL 50% binding response for antibody C and greater than 1.0 μg/mL for antibody J.

FIG. 16B is a graph showing the result of an ELISA analysis testing the binding property of anti AQP3 antibodies SC—F8 (circles), BC—H9 (gray squares), BC—B10 (triangles), and SC—B6 (exes)) compared to anti AQP3 antibodies (antibody C (diamonds) and antibody J (black squares)) to a peptide having the amino acid sequence of SEQ ID NO: 2, a Loop A peptide (FIG. 16B).

FIG. 17A is a graph showing the result of an ELISA analysis testing the binding property of increasing concentration (1 ng/mL, 10 ng/mL, 100 ng/mL, 1 μg/mL, or 10 μg/mL) of anti AQP3 antibodies of the present invention to mouse keratocytes (PAM212 cells). FIG. 17A shows the binding of BC—H9 and BC—B10 to PAM212 cells.

FIG. 17B is graph showing the result of an ELISA analysis testing the binding property of increasing concentration (1 ng/mL, 10 ng/mL, 100 ng/mL, 1 μg/mL, or 10 μg/mL) of anti AQP3 antibodies of the present invention to mouse keratocytes (PAM212 cells). FIG. 17B shows the binding of SC—F8 and SC—B6 to PAM212 cells.

FIG. 18A is a graph showing the result of an ELISA analysis testing the binding property of increasing concentration (1 ng/mL, 10 ng/mL, 100 ng/mL, 1 μg/mL, or 10 μg/mL) of anti AQP3 antibodies of the present invention to human keratocytes (HaCaT cells). FIG. 18A shows the binding of BC—H9 and BC—B10 to HaCaT cells.

FIG. 18B is a graph showing the result of an ELISA analysis testing the binding property of increasing concentration (1 ng/mL, 10 ng/mL, 100 ng/mL, 1 μg/mL, or 10 μg/mL) of anti AQP3 antibodies of the present invention to human keratocytes (HaCaT cells). FIG. 18B shows the binding of SC—F8 and SC—B6 to HaCaT cells.

FIG. 19 is a graph showing the result of an ELISA analysis testing the binding property of anti AQP3 antibodies SC—F8, BC—H9, BC—B10, and SC—B6 to mouse keratocytes (PAM212 cells) that have been transfected with an siRNA against AQP3 or a siRNA control.

FIG. 20 is a diagram showing the result of testing the functional inhibition effect of anti AQP3 antibodies SC—F8, BC—H9, BC—B10, and SC—B6, anti AQP3 antibody C at two concentrations 1 μg/mL and 10 μg/mL and a control antibody that does not bind to AQP3 on the hydrogen peroxide permeation function in mouse keratocytes (PAM212 cells).

FIG. 21 is a diagram showing the result of testing the functional inhibition effect of BC—B10 and SC—B6 at increasing concentrations (1 ng/mL, 10 ng/mL, 100 ng/mL, 1 μg/mL, or 10 μg/mL) on the hydrogen peroxide permeation function in mouse keratocytes (PAM212 cells).

FIG. 22 is a diagram showing the result of testing the functional inhibition effect of anti AQP3 antibodies SC—F8, BC—H9, BC—B10, and SC—B6 at two concentrations 1 μg/mL and 10 μg/mL on the hydrogen peroxide permeation function in human keratocytes (HaCaT cells). Also shown is a dashed line indicating that three of the clones inhibited H₂O₂ permeation approximately 50%, or greater, when compared to the control antibody.

FIG. 23 is a diagram showing the result of testing the functional inhibition effect of two of the anti AQP3 antibodies, BC—B10 and SC—B6, at increasing concentrations (1 ng/mL, 10 ng/mL, 100 ng/mL, 1 μg/mL, or 10 μg/mL) on the hydrogen peroxide permeation function in human keratocytes (HaCaT cells).

FIG. 24 is a graph showing the result of functional inhibition effect with and without the presence of AQP3 by anti AQP3 antibodies SC—F8, BC—H9, BC—B10, and SC—B6 on the hydrogen peroxide permeation function in mouse keratocytes (PAM212 cells). PAM212 cells that have been transfected with an siRNA against AQP3 (without AQP3) or a siRNA control (with AQP3).

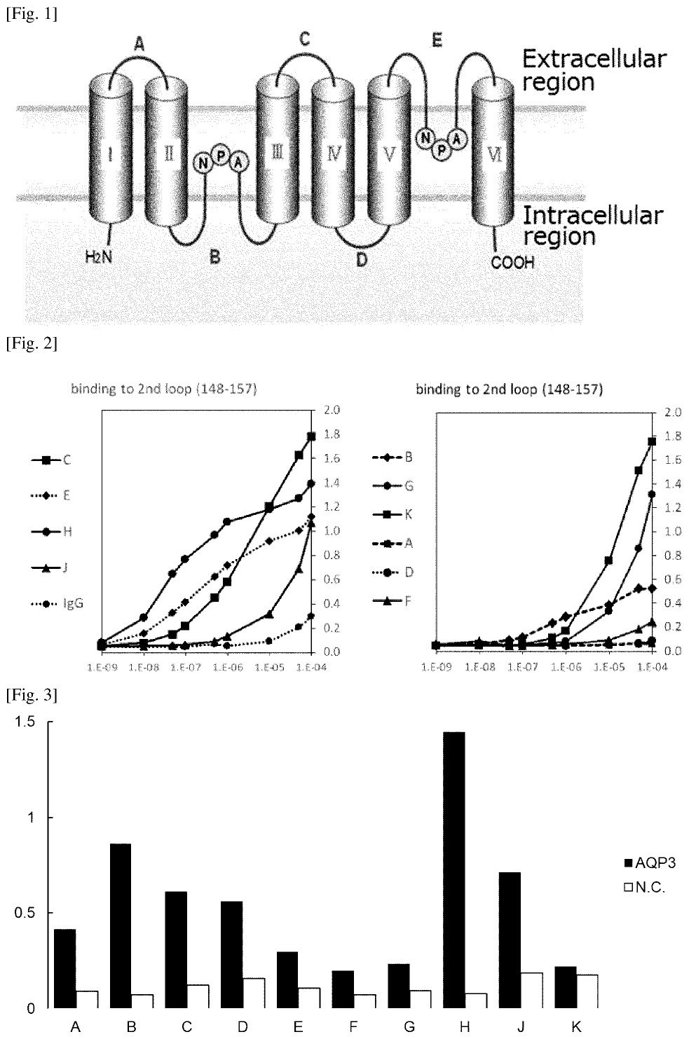

### DETAILED DESCRIPTION

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

To provide a solution for the Technical Problem addressed above, anti-AQP3 antibodies were produced using a novel antibody production method.

### DESCRIPTION OF EMBODIMENTS

(1) Preparation of an anti AQP3 antibody specifically recognizing extracellular domain of AQP3

Because there are three extracellular domains in AQP3, such as loop A, loop C, and loop E, by having at least one AQP3 fragment of them as an immunogen, a host animal can be immunized. In the case of human AQP3, in the polypeptide consisting of full-length 292 amino acid residues (UniProt accession: Q92482), positions 50 to 53 (loop A), positions 131 to 157 (loop C), and positions 210 to 244 (loop E; all positions represent the position from N-terminal side) form each of the extracellular domains. The immunogen is preferably an AQP3 fragment of loop C. Particularly preferably, a polypeptide composed of ten amino acid residues, which is the C-terminal part of loop C and adjacent to the boundary to the transmembrane domain IV, is used as an immunogen. The C-terminal part of loop C adjacent to the boundary to the transmembrane domain IV has the amino acid sequence ATYPSGHLDM (SEQ ID NO: 1) in both human and mouse.

Oligopeptides can be chemically synthesized by well-known standard methods. Furthermore, they can be simply obtained by using a custom-made synthesis service that is commercially available.

As for the immunogen, an oligopeptide itself can be used for immunization, or it is also possible that immunization can be carried out by using reconstituted membrane or recombinant body cells which provide a polypeptide containing the oligopeptide to a membrane. When the immunogen is prepared in the form of a transmembrane protein containing the oligopeptide part, the preparation is preferably carried out by using a baculovirus display method. In that case, a polypeptide containing the oligopeptide can be expressed on a membrane surface of baculovirus and immunization of a host animal can be carried out by using the baculovirus itself as an immunogen to induce an antibody. Those immunogens may be used for immunization either singly or a combination of them may be used simultaneously.

In some embodiments, the host animal is immunized with a peptide whose amino acid sequence consists of the amino acid sequence of SEQ ID NO:1 or SEQ ID NO:1 in combination with AQP3 overexpressing cells. For example, AQP3 overexpressing cells can be HaCaT cells, PAM212 cells, mouse macrophages, or HEK293 cells over-expres sing AQP3 or a combination thereof. In another embodiment, the AQP3 overexpressing cells are AQP3 overexpressing CHO cells, e.g., CHO cells expressing mouse or human AQP3 under the control of the CMV promoter. Exemplary vectors that can be used include pCMV6-AC (Origene sc322406) (human AQP3) and pCMV6-Entry-Myc-DDK (Origene MR203989) (mouse AQP3). In some embodiments, the AQP3 overexpressing cells comprise a combination of CHO cells over-expres sing mouse AQP3 CHO cells overexpressing human AQP3.

Preferred examples of the host animal to be immunized include, although not particularly limited, animals like mouse, rat, rabbit, guinea pig, sheep, goat, donkey, chicken, and camel. More preferably, the host animal is a mouse or a rat, and particularly preferably a mouse. For example, reference can be made to the methods described in WO 2015/179360 A. An anti-blood serum containing an anti AQP3 antibody can be produced by a well-known standard method. Anti AQP3 antibodies can be any class of the five kinds of an immunoglobulin molecules (IgG, IgM, IgA, IgD, and IgE). Anti AQP3 antibodies are preferably IgG or IgM, and more preferably IgG. Among the IgG subclasses, IgG2 has lower ADCC activity and IgG4 has lower CDC activity. As such, when it is desired to use an antibody having low cell damaging property, it is preferable to use, among IgGs, an antibody of subclass IgG2 or IgG4.

(2) Preparation of an Anti AQP3 Monoclonal Antibody (Anti AQP3 mAb)

An anti AQP3 mAb can be produced as a monoclonal antibody by cloning after fusion of antibody-producing cells obtained during a preparation process as described above in (1) with myeloma cells. Alternatively, according to a genetic engineering method, it can be produced by expressing the chemically-synthesized antibody gene in E. coli or the like. The method for fusing antibody-producing cells and myeloma cells, the method for screening desired cells from the cell group containing the fused cells, the method for monoclonizing the cells selected by screening, and the method for producing mAb from clones can be all carried out according to well-known standard methods. Synthesis of a desired mAb based on sequence information can be also carried out according to well-known standard methods. As it is described in detail in the examples that are given below, monoclonal antibodies that are representative examples of the anti AQP3 mAbs of the present invention have the amino acid sequences of the heavy chain and light chain CDRs or the amino acid sequences of the heavy chain and light chain variable regions that are specifically disclosed. A mAb can be also prepared as a non-secretion type recombinant mAb which consists of an amino acid sequence obtained by removing the signal sequence from each variable region of the heavy chain and light chain. The recombinant mAb with removed signal sequence can accumulate in a host cell without being secreted from the host cell expressing the recombinant mAb into a culture supernatant. The signal sequence can be predicted from the amino acid sequence information, and, for example, it can be predicted by using a software for predicting signal sequence. Exemplary software for predicting signal sequence include Signal P, PRORT II, and the like.

(3) Preparation of Inhibitory Anti AQP3 mAb

Among anti AQP3 antibodies, an antibody having an inhibitory activity for the function of AQP3 is referred herein to as an inhibitory anti AQP3 antibody. In the case of a monoclonal antibody, it is referred to as an inhibitory anti AQP3 mAb, in particular. Herein, the function of AQP3 indicates at least one activity selected from the group consisting of an activity of transporting (permeating) a low molecular weight material by AQP3, an activity of promoting cell proliferation of AQP3-expressing cells, and an activity of promoting cell migration of AQP3-expressing cells. Herein, the low molecular weight material indicates at least one material selected from the group consisting of water molecule, glycerol, and hydrogen peroxide. Presence or absence of the desired inhibitory activity of an anti AQP3 antibody can be determined by having, as an indicator, a decrease in at least one of the cell migration activity and/or cell proliferation activity by 10% or more, 20% or more, or 30% or more according to extracellular addition of a sufficient amount of the anti AQP3 antibody to the cells which constitutively express AQP3 (PAM212 cells, HaCaT cells, A431 cells, or the like) compared to a control without the addition. Alternatively, the determination can be made by having, as an indicator, a decrease in the hydrogen peroxide permeating activity of cells by 10% or more, 20% or more, or 30% or more, 40% or more, 50% or more, 60% or more according to extracellular addition of a sufficient amount of the anti AQP3 antibody to the cells which constitutively express AQP3 (mouse macrophage cells or the like) compared to a control without the addition.

(4) Functional Fragment of an Antibody

As long as sufficient specificity and affinity for AQP3 are exhibited, an antibody of the present invention is not necessarily required to maintain the whole structure of an immunoglobulin molecule, and it can be a functional fragment of the antibody (antigen binding fragment). Because the antigen binding property of an antibody is decided by a variable part of the antibody, the constant region part of an immunoglobulin molecule may not be necessarily present. As such, examples of a functional fragment of an antibody of the present invention include Fab, Fab′, F(ab′)2, which are a fragment consisting of a variable part of an immunoglobulin molecule, Fd obtained by removing VL from Fab, single-chain Fv fragment (scFv) and a dimer thereof, i.e. a diabody. A1-ternatively, a single domain antibody (sdAb) obtained by removing VL from scFv, or the like can be also used, but the functional fragment of the antibody is not limited to them.

A functional fragment of an antibody can be prepared by a known technique. For example, fragmentation can be carried out by an enzyme treatment of an immunoglobulin molecule. According to degradation of an immunoglobulin molecule with papain, a Fab is obtained. According to degradation with pepsin, a F(ab′)2 is obtained, and according to a reducing treatment of a F(ab′)2, a Fab′ is obtained. Furthermore, it is also possible, according to a genetic engineering technique, to produce a scFv by linking a heavy chain variable part (VH) to a light chain variable part (VL) of an antibody via a linker peptide with sufficient mobility.

(5) Antibody Labeled with Reporter Material

Depending on a case, an anti AQP3 antibody or a functional fragment thereof of the present invention is used in a state where it is labeled with a reporter material. The reporter material can be any kind as long as it can label the anti AQP3 antibody or a functional fragment thereof while they maintain a desired function. A material capable of generating a signal for quantitative measurement of the present of AQP3 is more preferable. Examples thereof include a radioactive isotope, a metal micro particle, an enzyme, a fluorescent material, and a luminescent material. When a radioactive isotope, a fluorescent material, or a luminescent material is used as a reporter material, the radioactivity, fluorescence, or luminescence generated from them can be quantitatively measured as a signal. When the reporter material is an enzyme, after application to a suitable substrate, the pigment that is finally generated, color, fluorescence, or luminescence derived from fluorescent material or luminescent material can be measured as a signal. Examples of radioactive isotopes include 3H and 125I. Examples of fluorescent materials include fluorescein and derivatives thereof (for example, FITC), tetramethyl rhodamine (TAMRA) and derivatives thereof (for example, TRITC), Cy3, Cy5, Texas Red, phycoerythrin (PE), and quantum dots. Examples of luminescent materials include a luminol derivative, an acridinium derivative, aequorin, and a ruthenium complex. Examples of metal micro particles include gold nano particles and nano particles composed of an alloy of gold and platinum. Examples of reporter enzymes include horseradish peroxidase (HRP), β-galactosidase (β-GAL), alkali phosphatase (ALP), glucose oxidase (GOD), luciferase, and aequorin. By using each enzyme in combination with a suitable substrate, analysis based on light-emission method, colorimetric method, or fluorescence method can be made. For a quantitative analysis, an antibody or a functional fragment thereof of the present invention, which is labeled with a reporter material, is preferably used.

(6) Antibody Immobilized on Solid Support

Depending on a case, an anti AQP3 antibody or a functional fragment thereof of the present invention can be used in a state where it is immobilized on a solid support. The solid support can be any material as long as it can immobilize an antibody or a functional fragment thereof while they remain in a state of maintaining a desired activity. It is preferably a material composed of an inactive material which does not have any influence on the biological analysis using an antibody. Examples of solid supports include a micro plate, a glass plate, a plastic plate, a syringe, a vial, a column, a magnetic particle, a micro bead made of resin, a porous membrane, a porous carrier, and a microchip. The micro plate, syringe, vial, column, and microchip are all preferably made of an inactive resin. Solid supports can be also made of glass.

(7) Antibody Specifically Binding to AQP3 Derived from Human and/or Mouse

An anti AQP3 antibody or a functional fragment thereof of the present invention binds to the extracellular domain of AQP3, in particular, loop C (second extracellular domain) in some embodiments. The amino acid sequence of loop C exhibits high conservation among biospecies. Both the amino acid sequence of human loop C and the amino acid sequence of mouse loop C (positions 131 to 157 from the N-terminal side for both human and mouse) have high homology as it is described below.

Human:
(SEQ ID NO: 65)
ADNQLFVSGPNGTAGIFATYPSGHLDM
Mouse:
(SEQ ID NO: 66)
ANNELFVSGPNGTAGIFATYPSGHLDM

Due to the above reason, an anti AQP3 antibody or a functional fragment thereof of the present invention, which binds to loop C as the extracellular domain, is highly likely to bind specifically to human AQP3 and also mouse AQP3. In some aspects, the present invention relates to antibodies which can be obtained by using the polypeptide (oligopeptide) composed of ten amino acid residues at the C-terminal side of loop C as an immunogen. The oligopeptide composed of ten amino acid residues has an amino acid sequence consisting of ATYPSGHLDM (SEQ ID NO: 1), and the human sequence and mouse sequence are in complete match in that part. Due to this reason, it is highly likely that an anti AQP3 antibody or a functional fragment thereof which is obtained according to the examples of the present invention not only specifically recognizes human AQP3 but also specifically recognizes mouse AQP3. Actually, according to the testing performed on individual mAbs described in the examples, it appears that mAbs of the disclosure can generally recognize both of them. Furthermore, an inhibitory anti AQP mAb of the present invention and a functional fragment thereof can in some embodiments inhibit the function of AQP3 for both human AQP3 and mouse AQP3. According to certain embodiments, the same is true for antibodies generated from other oligopeptides of Loop C and antibodies generated from oligopeptides of Loop A and E.

In some embodiments, anti AQP3 antibodies and functional fragments thereof do not specifically bind to one or more human aquaporins other than AQP3, for example, one or more of AQP0 (Accession no. NP_036196.1), APQ1 (Accession no. NP_932766.1), AQP2 (Accession no. NP_000477.1), AQP4 (Accession no. NP_001641.1), AQP5 (Accession no. NP_001642.1), AQP6 (Accession no. NP_001643.2), AQP7 (Accession no. NP_001161.1), AQP8 (Accession no. NP_001160.2), AQP9 (Accession no. NP_066190.2), AQP10 (Accession no. NP_536354.2), AQP11 (Accession no. NP_766627.1), and AQP12 (Accession no. NP_945349.1).

(8) Variable Region of Antibody Molecules and Complementarity-Determining Regions in Variable Regions

An immunoglobulin molecule is a hetero tetramer molecule which is basically composed of two heavy chain polypeptides and two light chain polypeptides. Each of the heavy chain and light chain contains a variable region and a constant region. The heavy chain variable region and light chain variable region of an antibody consist of three CDRs (complementarity-determining regions) and four FRs (framework regions), and FR1, CDR1, FR2, CDR2, FR3, CDR3, and FR4 are arranged in the order, from the amino terminal to the carboxy terminal. When the amino acid sequence information of an antibody molecule is determined by a known technique, location of a variable region or a constant region can be predicted based on the sequence information. Furthermore, predicting the sequence of CDR1, CDR2, and CDR3 in a variable region can be also similarly carried out by known methods.

(9) Preparation of Antibody Molecules

A representative anti AQP3 mAb of the present invention is an mAb of which heavy chain variable region consists of the amino acid sequence represented by SEQ ID NO: 15 and light chain variable region consists of the amino acid sequence represented by SEQ ID NO: 16, an mAb of which heavy chain variable region consists of the amino acid sequence represented by SEQ ID NO: 45 and light chain variable region consists of the amino acid sequence represented by SEQ ID NO: 46, or an mAb of which heavy chain variable region consists of the amino acid sequence represented by SEQ ID NO: 49 and light chain variable region consists of the amino acid sequence represented by SEQ ID NO: 50.

An anti AQP3 antibody of the present invention can be produced as a monoclonal antibody by, after cloning the antibody gene from hybridoma or artificially syn-thesizing the antibody gene based on the amino acid sequence information of the antibody polypeptide, introducing the antibody gene to a suitable expression vector, and introducing the vector to a host using a gene recombination technique.

In that case, a promoter, an enhancer, a polyadenylation signal, or the like can be suitably arranged in the vector. As for the vector, any vector can be used as long as it uses a replicable host cells like bacteria, yeast, and animal cells, and a commercially available vector can be suitably used depending on a host. The expression vector can be introduced to a host cell by a known method for transforming the host cells. Examples of the method include an electroporation method, a DEAE-dextran method, and a calcium phosphate method.

The host cell is not particularly limited, but a eukaryotic cell is preferably used. Examples thereof include yeast and cultured cells derived from an animal (HEK293 cells, CHO cells, COS cells, and MEF, etc.).

Purification of a produced antibody can be carried out by using a method for separation and purification that is generally employed for proteins. For example, it can be suitably carried out by suitably combining affinity chromatography, other chromatography, filtration, ultrafiltration, salting-out, dialysis, and the like.

(10) Modified Products of Antibodies

An anti AQP3 mAb of the present invention may be a sequence-modified product of an antibody having the amino acid sequences described in the above sections. For example, by having an antibody of which heavy chain variable region consists of a given amino acid sequence and light chain variable region consists of a given amino acid sequence as a starting point for modification, and within a range in which the specific binding property to the extracellular domain of AQP3 is substantially maintained (within a range in which a specific binding property substantially equivalent to the specific binding property of the original antibody is maintained), a modification may be present within each variable region of the heavy chain and light chain. In each of the amino acid sequence described above, it is also possible that one or several, for example one to ten, preferably one to five, more preferably one or two, and even more preferably one amino acid residue is deleted, substituted, inserted, or added. Furthermore, when calculation is made by using a tool like BLAST, the modification may be present within a range in which there is sequence homology of at least 85% or more, preferably 90% or more, more preferably 95% or more, and particularly preferably 97% or more. However, for any modified product, there is preferably no modification of the amino acid sequence of the CDRs (such that each CDR has the same amino acid sequence as that of the antibody before modification).

It is widely accepted that the CDR sequence is a major factor for determining an epitope of an antibody. An anti AQP3 mAb of the present invention preferably has, even for the sequence-modified product described above, completely preserved CDRs present in total number of 6 as it is included in the heavy chain and light chain. As such, it is reasonably expected to have a specific binding property for the same epitope as the anti AQP3 mAb before modification. Furthermore, as long as it binds to the same epitope, it is also reasonably expected that, even when the anti AQP3 mAb is the above described sequence-modified product, it has the activity of inhibiting the function of AQP3 as the antibody before modification.

(11) Chimeric Antibodies and Humanized Antibodies

An anti AQP3 mAb of the present invention can be an artificially-modified gene recombination type antibody for the purpose of reducing the heteroantigenicity to a human or the like. Examples of those antibodies include a chimeric antibody and a humanized antibody. These modified antibodies can be produced by known methods.

A chimeric antibody can be prepared by linking the DNA encoding the variable region (V) of an anti AQP3 mAb of the present invention to the DNA encoding a constant (C) region of a human antibody, introducing the resultant construct to an expression vector, and introducing the vector to a host.

A humanized antibody can be obtained by grafting CDRs of an antibody of a mammal other than a human, such as CDRs of a mouse antibody, to a human acceptor antibody (CDR grafting). Production thereof can be suitably carried out by applying a common technique for gene recombination. For example, it is possible that a DNA sequence designed to encode an amino acid sequence for linking each CDR of a mouse anti AQP3 mAb and a framework region of a human antibody is synthesized by PCR method by using several oligonucleotides as a primer, which have been prepared such that they have an overlapped region at terminal regions of both the CDR and FR. For example, it can be carried out by a method described in WO 98/13388 A. The FR of the variable region of a human antibody can be obtained from published DNA data base or the like.

As for the constant region of a chimeric antibody and a humanized antibody, the constant region of a human antibody can be used. For example, Cγ1, Cγ2, Cγ3, and Cγ4 are preferably used for the heavy chain while Cκ and Cλ are preferably used for the light chain.

Because chimeric antibodies and humanized antibodies have reduced heteroantigenicity in the human body, they have long half-life in a living body of a human and are useful as an effective ingredient of the pharmaceutical composition of the present invention (agent for prevention and/or treatment). Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; EP519596; Padlan, 1991, Mol. Immunol., 28:489-498; Studnicka et al, 1994, Prot. Eng. 7:805-814; Roguska et al, 1994, Proc. Natl. Acad. Sci. 91:969-973; and U.S. Pat. No. 5,565,332.

In some embodiments, the anti AQP3 antibodies and functional fragments thereof can be antibodies or antibody fragments whose sequence has been modified to alter at least one constant region-mediated biological effector function relative to the corresponding wild type sequence.

For example, in some embodiments, an anti AQP3 antibody of the present invention can be modified to reduce at least one constant region-mediated biological effector function relative to an unmodified antibody, e.g., reduced binding to the Fc receptor (FcγR). FcγR binding can be reduced by mutating the immunoglobulin constant region segment of the antibody at particular regions necessary for FcγR interactions (see e.g., Canffeld and Morrison, 1991, J. Exp. Med. 173:1483-1491; and Lund et al., 1991, J. Immunol. 147:2657-2662). Reduction in FcγR binding ability of the antibody can also reduce other effector functions which rely on FcγR interactions, such as opsonization, phagocytosis and antigen-dependent cellular cytotoxicity (“ADCC”).

In other embodiments, an anti AQP3 antibody of the present invention can be modified to acquire or improve at least one constant region-mediated biological effector function relative to an unmodified antibody, e.g., to enhance FcγR interactions (see, e.g., US 2006/0134709). For example, an anti AQP3 antibody of the present invention can have a constant region that binds FcγRIIA, FcγRJIB and/or FcγRIIIA with greater affinity than the corresponding wild type constant region.

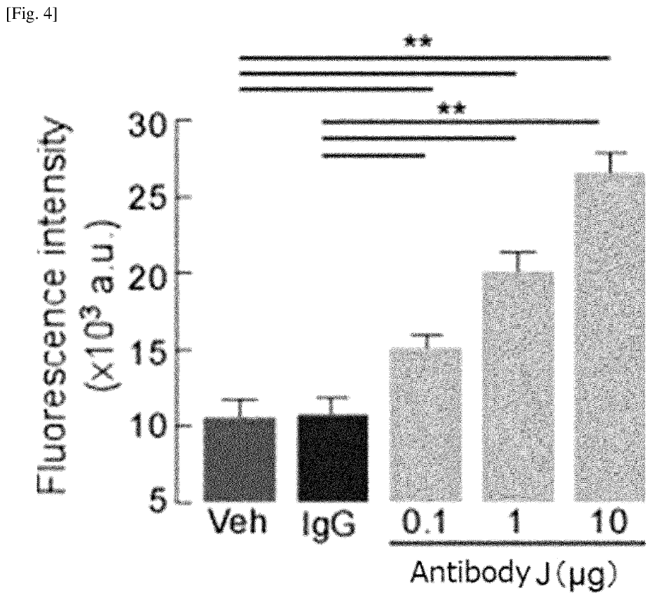

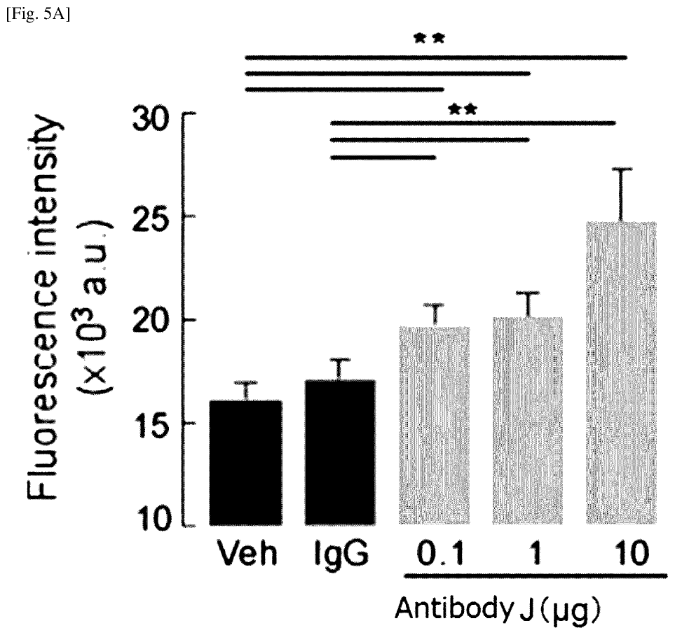

Thus, antibodies of the present invention can have alterations in biological activity that result in increased or decreased opsonization, phagocytosis, or ADCC. Such alterations are known in the art. For example, modifications in antibodies that reduce ADCC activity are described in U.S. Pat. No. 5,834,597. An exemplary ADCC lowering variant corresponds to “mutant 3” shown in FIG. 4 of U.S. Pat. No. 5,834,597, in which residue 236 is deleted and residues 234, 235 and 237 (using EU numbering) are substituted with alanines.

(12) Reagents for Detecting AQP3

From the viewpoint that an anti AQP3 antibody or a functional fragment thereof of the present invention has an ability of specifically binding to AQP3, a composition containing the antibody or a functional fragment thereof can be provided. This composition can be provided as a reagent for detecting AQP3. Herein, an anti AQP3 antibody or a functional fragment thereof to be contained in a reagent may also be one which is labeled with a reporter material as it has been described in above (5). When it is labeled with a reporter material, detection can be carried out without using a secondary antibody. As another embodiment, an antibody or a functional fragment thereof to be contained in a reagent may be bound or adsorbed onto a solid support such as magnetic micro particles. In a case in which the anti AQP3 antibody or a functional fragment thereof of the present invention is contained as a solution in the reagent, the concentration thereof can be suitably set depending on the purpose of the reagent or mode of use. For example, it can be set within a range of 1 ng/mL to 10 mg/mL, 100 ng/mL to 1 mg/mL, or 1 μg/mL to 300 μg/mL. Furthermore, although the reagent may be used as a stock solution by itself, it can also be used in a diluted state (10 times to 10,000 times) depending on the purpose. As for the solvent, water or a buffer solution can be suitably used.

(13) Reagents for Identification, Separation, and Purification of AQP3-Expressing Cells

An anti AQP3 antibody or a functional fragment thereof of the present invention specifically recognizes and binds to the extracellular domain of AQP3, more specifically, the epitope within loop C in some embodiments. From the viewpoint that it can bind to the extracellular domain of an AQP3 molecule, it can be also used for a system in which living cells are employed as a sample. Even for a case of carrying out immunohistological staining, it is not necessary to perform fixing or dialysis of tissue or cells. Accordingly, regardless of the state of cells to be a sample, an anti AQP3 antibody or a functional fragment thereof of the present invention can be used for the identification of AQP3-expressing cells. In particular, when isolated living cells like hematocyte cells are employed as a sample, an anti AQP3 antibody or a functional fragment thereof of the present invention can be used for separation or purification of the AQP3-expressing cells according to combination with a suitable instrument like a flow cytometer. When it is used for separation or purification of the AQP3-expressing cells, an anti AQP3 antibody or a functional fragment thereof labeled with a reporter material as described in above (5) are suitably used. As for the reporter material, a fluorescent pigment is preferable. Examples thereof include FITC, PE/RD1, ECD, PC5, PC7, and APC/Cy3. Alternatively, for separation or purification of the AQP3-expressing cells, an anti AQP3 antibody or a functional fragment thereof immobilized onto a solid phase such as magnetic micro particles can be also used. After binding to the anti AQP3 antibody or a functional fragment thereof immobilized onto a solid phase, the AQP3-expressing cells can be specifically separated by utilizing magnetic force or the like. After the separation, the antibody or a functional fragment thereof can be dissociated from the cells based on adjustment of salt strength or the like. As such, according to this order, the separation or purification of the AQP3-expressing cells can be completed. For the identification, separation, or purification of the AQP3-expressing cells, the composition containing the anti AQP3 antibody or a functional fragment thereof of the present invention is provided as a reagent for detecting AQP3. The reagent may be produced and used as it is described in above (12).

(14) Reagents for Measuring Expression Amount of AQP3

An anti AQP3 antibody or a functional fragment thereof of the present invention can be used as a component of the reagent for detecting AQP3 as described in above (12). Herein, if the anti AQP3 antibody or a functional fragment thereof is labeled with a reporter material as described in above (5) and the reporter material generates a signal allowing quantitative measurement, not only the presence or absence of AQP3 as a target but also the expression amount of AQP3 can be quantitatively measured. Furthermore, even in a case in which an anti AQP3 antibody labeled with a reporter material or a functional fragment thereof is not used, by using in combination a secondary antibody that is labeled with a reporter material which generates a signal allowing quantitative measurement, an anti AQP3 antibody or a functional fragment thereof of the present invention can be used for the measurement of the expression amount of AQP3. For this purpose, a composition containing an anti AQP3 antibody or a functional fragment thereof of the present invention is provided as a reagent for measuring the expression amount of AQP3. The reagent may be suitably produced and used as it is described in the example of above (12).

(15) Antibody Drug Conjugates

The present invention provides antibody drug conjugates (ADCs) comprising an anti AQP3 antibody of the present invention or functional fragment thereof conjugated to a cytotoxic agent. Linkers and processes for making ADCs are known in the art and can be used to make an ADC of the present invention. See, e.g., Tsuchikama and An, 2018, Protein & Cell, 9(1):33-46; Deonarain et al., 2015, Expert Opin Drug Discov. 10(5):463-81; Singh et al., 2015, Pharm Res. 2015 November; 32(11):3541-71. The ADCS of the disclosure can be included in pharmaceutical compositions for use in treating cancer.

Exemplary cytotoxic agents include, for example, auristatins, camptothecins, calicheamicins, duocarmycins, etoposides, maytansinoids (e.g., DM1, DM2, DM3, DM4), taxanes, benzodiazepines (e.g., pyrrolo[1,4]benzodiazepines, indolinobenzodi-azepines, and oxazolidinobenzodiazepines including pyrrolo[1,4]benzodiazepine dimers,

indolinobenzodiazepine dimers, and oxazolidinobenzodiazepine dimers) and vinca alkaloids.

Techniques for conjugating therapeutic agents to proteins, and in particular to antibodies, are well-known. (See, e.g., Alley et al., 2010, Current Opinion in Chemical Biology 14: 1-9; Senter, 2008, Cancer J., 14(3): 154-169.) Typically, the therapeutic agent is conjugated to the antibody via a linker unit. The linker unit can be cleavable or non-cleavable. For example, the therapeutic agent can be attached to the antibody with a cleavable linker that is sensitive to cleavage in the intracellular environment of an AQP3 expressing cancer cell but is not substantially sensitive to the extracellular environment, such that the conjugate is cleaved from the antibody when it is internalized by the AQP3 expressing cancer cell (e.g., in the endosomal, lysosomal environment, or in the caveolear environment). In another example, the therapeutic agent can be conjugated to the antibody via a non-cleavable linker and drug release is by total antibody degradation following internalization by the AQP3 expressing cancer cell.

Typically, the ADC will comprise a linker region between the cytotoxic agent and the anti AQP3 antibody. As noted supra, typically, the linker can be cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, 1999, Pharm. Therapeutics 83:67-123). Most typical are peptidyl linkers that are cleavable by enzymes that are present in AQP3 expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide). The linker can also be a carbohydrate linker, including a sugar linker that is cleaved by an intracellular glycosidase (e.g., a glucuronide linker cleavable by a glucuronidase).

The linker also can be a non-cleavable linker, such as an maleimido-alkylene- or maleimide-aryl linker that is directly attached to the therapeutic agent and released by proteolytic degradation of the antibody.

The anti AQP3 antibody can be conjugated to the linker via a heteroatom of the antibody. These heteroatoms can be present on the antibody in its natural state or can be introduced into the antibody. In some aspects, the anti AQP3 antibody will be conjugated to the linker via a nitrogen atom of a lysine residue. In other aspects, the anti AQP3 antibody will be conjugated to the linker via a sulfur atom of a cysteine residue. The cysteine residue can be naturally-occurring or one that is engineered into the antibody. Methods of conjugating linkers and drug-linkers to antibodies via lysine and cysteine residues are known in the art.

Exemplary antibody-drug conjugates include auristatin based antibody-drug conjugates (i.e., the drug component is an auristatin drug). Auristatins bind tubulin, have been shown to interfere with microtubule dynamics and nuclear and cellular division, and have anticancer activity. Typically the auristatin based antibody-drug conjugate comprises a linker between the auristatin drug and the anti AQP3 antibody. The linker can be, for example, a cleavable linker (e.g., a peptidyl linker, a carbohydrate linker) or a non-cleavable linker (e.g., linker released by degradation of the antibody). Auristatins include MMAF, and MMAE. The synthesis and structure of exemplary auristatins are described in U.S. Pat. Nos. 7,659,241, 7,498,298, 2009-0111756, 2009-0018086, and 7,968, 687.

Other exemplary antibody-drug conjugates include maytansinoid antibody-drug conjugates (i.e., the drug component is a maytansinoid drug), and benzodiazepine antibody drug conjugates (i.e., the drug component is a benzodiazepine (e.g., pyrrolo[1,4]benzodiazepine dimers (PBD dimer), indolinobenzodiazepine dimers, and oxazolidinobenzodiazepine dimers)).

(16) Kits Obtained by Including a Composition Containing an Anti AQP3 Antibody or Functional Fragment Thereof

As described in above (12) to (14), by using an anti AQP3 antibody or a functional fragment thereof of the present invention, a reagent for detecting AQP3, a reagent for identification, separation, or purification of AQP3-expressing cells, and a reagent for measuring an expression amount of AQP3 can be prepared. In accordance with respective purpose, those reagents can be used for forming a kit, together with an additional component. The kit is suitably combined with constitutional elements such as AQP3 or a fragment thereof as a positive control, AQP3 with known concentration as a standard material, a secondary antibody, an enzyme substrate, a co-factor, an assistant component, a non-specific protein sample as a negative control, a buffer solution, a preservative, a diluent, a user guide book, or the like. A buffer solution for blocking or washing can be also added as a suitable constitutional element of the kit.

(17) Compositions Containing an Inhibitory Anti AQP3 mAb or Functional Fragment Thereof and Compositions as AQP3 Inhibitors

An anti AQP3 antibody, a functional fragment thereof, or ADC of the present invention specifically recognizes and binds to the extracellular domain of AQP3, in particular, the epitope in loop C in some embodiments. As it is specifically described in the examples given below, an anti AQP3 mAb of the present invention which binds to the epitope can inhibit at least one function of AQP3 such as the channel function (for example, hydrogen peroxide permeating property) of AQP3 or function of promoting cell proliferation of AQP3 in AQP3-expressing cells. Namely, an anti AQP3 antibody of the present invention can be regarded as an inhibitory anti AQP3 antibody. As such, it is possible to provide a composition which contains an inhibitory anti AQP3 mAb of the present invention or a functional fragment thereof. Furthermore, this composition can be used as an AQP3 inhibitor.

(18) Compositions for Treatment of Cancer

An increased expression level of AQP3 is confirmed in each of skin cancer, colorectal cancer, cervical cancer, liver cancer, lung cancer, esophageal cancer, kidney cancer, stomach cancer, tongue cancer, and the like. Furthermore, as it is described in the examples given below, proliferation of human cancer cell lines, in which AQP3 is expressed, can be inhibited. Accordingly, a composition containing an inhibitory anti AQP3 mAb of the present invention or a functional fragment thereof, an ADC of the present invention, or an AQP3 inhibitor can be used as a composition for treating any one of the above cancers. Furthermore, as it has been suggested that the function of AQP3 is associated with a progress level of cancer, tumor angiogenesis, infiltration property, metastasis, and energy metabolism of cancer tissues, or the like, the composition for treating cancer can be also regarded as a composition for inhibiting cancer proliferation, a composition for inhibiting angiogenesis in cancer, a composition for inhibiting cancer infiltration, and/or a composition for inhibiting/preventing cancer metastasis.

A composition for treating cancer of the present invention can be prepared in a formulation such as a solution for injection or the like. Basically, such a composition for treating cancer can be systemically administered by injection or dropwise addition. However, in a case in which it is used for the purpose of treating cancer or preventing metastasis or the like, topical administration can be also carried out. Those preparations can be prepared by known methods. When it is prepared in a preparation for injection, for example, production can be carried out by dissolving or diluting an inhibitory anti AQP3 mAb of the present invention or a functional fragment thereof, or an ADC of the present invention, which has been aseptically preserved, in water, physiological saline, or buffer solution for injection.

An effective dose of an inhibitory anti AQP3 mAb of the present invention or a functional fragment thereof or an ADC of the present invention, which becomes an effective ingredient of the treatment composition of the present invention, suitably varies depending on various conditions including a state, a symptom, or the like of a patient. In general, a single dose is determined within a range of 0.1 to 10 mg of anti AQP3 mAb/kg of body weight, and it is administered by subcutaneous injection, intravenous injection, intraperitoneal injection, or the like. The administration interval also suitably varies depending on various conditions including a state, a symptom, or the like of a patient. In general, the administration is made once for 1 to 4 weeks, but it is also possible that, after having several weekly administrations, no administration is made for a certain period, or, after one to several initial administrations, administration can be continued at the same pace while the dose is cut down to half or the like.

(19) Compositions for Preventing and/or Treating Skin Disorders

A composition containing an inhibitory anti AQP3 mAb of the present invention or a functional fragment thereof or the AQP3 inhibitor can be used, based on a mechanism of inhibiting the function of AQP3 in cells of skin tissues like keratinocyte, as a composition for preventing and/or treating a skin disorder. Specific examples of the skin disorder include psoriasis, actinic keratosis, ichthyosis, and seborrheic dermatitis. Other than that, for curing or ameliorating keratinocyte proliferative skin abnormality, a composition containing an inhibitory anti AQP3 mAb of the present invention or a functional fragment thereof or a composition for treatment of the present invention which is obtained by containing an AQP3 inhibitor can be used.

(20) Compositions for preventing and/or treating inflammatory disorders

A composition containing an inhibitory anti AQP3 mAb of the present invention or a functional fragment thereof or an AQP3 inhibitor can be used, based on a mechanism of reducing an inflammatory response according to inhibition of the function of AQP3, as a composition for preventing and/or treating an inflammatory disorder. Specific examples of the inflammatory disorder include atopic dermatitis, psoriasis, asthma, and chronic obstructive lung disease, and hepatitis. Examples of the hepatitis include acute hepatitis and acute liver disorder. Other than that, for preventing, curing, or ameliorating an inflammatory disorder accompanying increased expression of AQP3, a composition containing an inhibitory anti AQP3 mAb of the present invention or a functional fragment thereof or a composition for preventing and/or treating an inflammatory disorder obtained by containing the AQP3 inhibitor can be used.

(21) Compositions for Alleviating Abnormality in Bowel Movement

It is widely known that AQP3 is expressed in intestinal epithelial cells, and it is suggested that the expression level of AQP3 has an influence on the transport amount of moisture inside and outside an intestine. Specifically, it is suggested that the reduced expression level of AQP3 can cause diarrhea by increasing the moisture inside an intestine, while the increased expression level of AQP3 can cause constipation by reducing the moisture inside an intestine. As such, a composition containing an inhibitory anti AQP3 mAb of the present invention or a functional fragment thereof or an AQP3 inhibitor can be used, based on a mechanism of inhibiting the function of AQP3, as a composition for alleviating an abnormality in bowel movement, in particular, as a composition for alleviating constipation. The composition may be prepared and used in the form of an enteric tablet or a suppository, for example. The enteric tablet or suppository can be suitably prepared by a known method. It is not necessary to carry out the administration continuously or periodically, and it can be carried out with a suitable interval depending on a change in symptoms or the like.

(22) Preparation of Compositions for Preventing and/or Treating Skin Disorders or InFlammatory Disorders of the Present Invention

An inhibitory anti AQP mAb of the present invention or a functional fragment thereof can be provided as, together with a pharmaceutically acceptable carrier or the like, a composition for prevention and/or treatment. Also, for a case in which a skin disorder or an inflammatory disorder is a subject, it can be basically and suitably prepared as a pharmaceutical composition (composition for prevention and/or treatment) like the composition for treating cancer that is described in above (18). The pharmaceutical composition can have a formulation like injection solution or the like. It may also have the form like aqueous solution, suspension, or emulsion. The pharmaceutical composition may contain a pharmaceutically acceptable diluent, aid, carrier, or the like including salts, buffering agents, adjuvants, or the like. Those preparations can be prepared by known methods. When it is produced in the form of a preparation for injection, the production can be made by dissolving or diluting a dried product or a preserved solution of the inhibitory anti AQP mAb or a functional fragment thereof, which has been aseptically preserved, with physiological saline or a buffer solution for subcutaneous injection or intravenous injection. Alternatively, it is also possible to enhance the water solubility by encapsulating the inhibitory anti AQP mAb or a functional fragment thereof by cyclodextrins.

(23) Assistant Components for Compositions for Preventing and/or Treating Skin Disorders or Inflammatory Disorders of the Present Invention

A composition containing an inhibitory anti AQP3 mAb of the present invention or a functional fragment thereof, or a composition for prevention or treatment containing an inhibitory anti AQP3 mAb may have a possibility of developing aggregation or precipitation of the anti AQP3 mAb or a functional fragment thereof, as it is often presented as a problem when other antibody preparations are developed while the preparation is a liquid preparation and concentration of the effective ingredient is high or the like. For the purpose of preventing the aggregation or precipitation, one or more than one assistant components may be included in the composition. Examples of the assistant components include saccharides such as monosaccharides, disaccharides, or oligosaccharides, sugar alcohols, salts, and surfactants. More specific examples thereof include sucrose, sodium chloride, and polyoxyethylene sorbitan monolaurate.

(24) Administration Forms of Compositions for Preventing and/or Treating Skin Disorders or Inflammatory Disorders of the Present Invention

An effective dose of an inhibitory anti AQP mAb or a functional fragment thereof, which becomes an effective ingredient of a composition for prevention and/or treatment of the present invention, suitably varies depending on various conditions including a state, a symptom, or the like of a patient. The administration dose suitably varies depending on various conditions including a state, a symptom, or the like of a patient. However, the dose as exemplified in the above (18) can be set, for example. The administration interval can be also set similar to the example of the above (18), but it is not necessary to carry out the administration continuously or periodically, and it can be carried out with a suitable interval depending on a change in symptoms or the like. It is needless to say that plural administrations would not be necessary if healing or remission is achieved by single administration. When there is recurrence or worsening of symptoms, the administration can be initiated again.

The administration period can be suitably adjusted depending on a disease condition of a patient. Although the administration dose during the administration period can be suitably adjusted, it is preferable that a constant amount is continuously administered or it is preferable to have administration form in which, after administration of relatively high dose only at initial administration stage, a shift to constant administration of less amount for maintenance is made.

### EXAMPLES

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the examples making apparent to those skilled in the art how the several forms of the invention may be embodied in practice.

### Example 1

Sequence Determination of Oligopeptide Used as Immunogen

To obtain an anti AQP3 antibody which specifically recognizes the extracellular domain of AQP3, the inventors of the present invention conducted multiple computer modeling studies on the structure of AQP3, in particular, the structure of loop A, loop C, and loop E constituting the extracellular domain, and, as a result, selected as an immunogen a fragment (oligopeptide) composed of the amino acid sequence of SEQ ID NO: 1, which constitutes a part of loop C (extracellular second loop). The amino acid sequence of SEQ ID NO: 1 is a sequence which corresponds to positions 148 to 157 of the human AQP3 polypeptide, and it is composed of ten amino acid residues at the C-terminal side of loop C that are adjacent to the boundary to the transmembrane domain IV.

### Example 2

Generation of Anti-AQP3 Antibodies in Mouse

An oligopeptide whose amino acid sequence consists of the amino acid sequence of SEQ ID NO: 1 was produced as a synthetic peptide. Furthermore, cells which overexpress the AQP3 polypeptide including that amino acid sequence (AQP3-overexpressing cells) were separately produced. Then, the synthetic peptide was combined with AQP3-overexpressing cells, and used as an immunogen.

A suspension of the above immunogen was immunized together with an adjuvant into the abdominal cavity of a mouse of the C57BL/6 line. After that, immune cells were collected from the immunized mouse and the antibody gene phage library was constructed. The phage library was introduced to CHO—K1 cells, and the recombinant antibodies were displayed in the cell membrane of the transformed CHO—K1 cells. Initial patterning was also carried out by using the transformed cells and the synthetic peptide, and patterning using AQP3-solubilizing protein was carried out subsequently. Using several screenings, AQP3-binding colonies were selected. Finally, clones having AQP3-specific binding activity were immunoglobulized (IgG) to obtain ten clones and ten anti AQP3 mAb (antibodies A, B, C, D, E, F, G, H, J, and K) that are derived from those 10 clones.

When an oligopeptide derived from loop E was used as an immunogen, a clone exhibiting a significant binding activity for AQP3 was not obtained.

### Example 3

Binding Property of Anti AQP3 Antibodies a, B, C, D, E, F, G, H, J, and K to AQP3

A. Antibody Binding to Immunogen Peptide

Binding of antibodies A, B, C, D, E, F, G, H, J, and K to the peptide used for immunization (SEQ ID NO:1) was tested in an ELISA assay. Results are shown in FIG. 2. Antibodies B, C, E, G, H, J, K were observed to bind the peptide. Antibodies A, D, and F, in contrast to the other antibodies, did not strongly bind the peptide in this assay. Thus, antibodies A, D, and F may therefore bind to AQP3 at a different epitope. B. Antibody binding to AQP3 containing cell lysate measured by ELISA

Cell lysate from HEK293T cells overexpressing mouse AQP3 and a myc-biotinylated tag was used in an ELISA assay to measure the binding of antibodies A, B, C, D, E, F, G, H, J, and K to AQP3. Cell lysate from HEK293T cells overexpressing the myc-biotinylated tag but not AQP3 was used as control. Results are shown in FIG. 3. Each of antibodies A, B, C, D, E, F, G, H, J, and K showed binding to AQP3. C. Antibody binding to AQP3 expressing cells

By using mouse epithelial cells (PAM212), mouse macrophage cells, human epithelial cells (HaCaT), and HEK293 cells as AQP3-expressing cells, the binding properties of the anti AQP3 antibodies A, B, C, D, E, F, G, H, J, and K to cells were measured.

PAM212 and macrophage cells were reacted with each anti AQP3 antibody (0.1, 1, or 10 μg/mL) at 4° C. for 1 hour. After washing the cells, a fluorescent-labeled secondary antibody was added and the reaction was allowed to occur additionally for 1 hour (4° C.). By measuring the fluorescence intensity, the binding property of each anti AQP3 antibody to cells was obtained.

The result obtained by using the mouse macrophage cells and antibody J is shown in FIG. 4.

The testing was also carried out using solvent (Veh) or a non-specific IgG (IgG) controls. In FIG. 4, the vertical axis represents fluorescence intensity, and the mean fluorescence intensity of each sample is represented by bar height together with standard error. From all cases in which antibody J was used at any concentration of 0.1, 1, and 10 μg/mL, significantly increased fluorescence intensity was recognized compared to the controls (Veh and IgG) (in the drawing, ** represents the presence of a significant difference of P<0.01). It was found that antibody J specifically recognizes the mouse AQP3 on cell surface so that antibody J and mouse macrophage cells bind to each other.

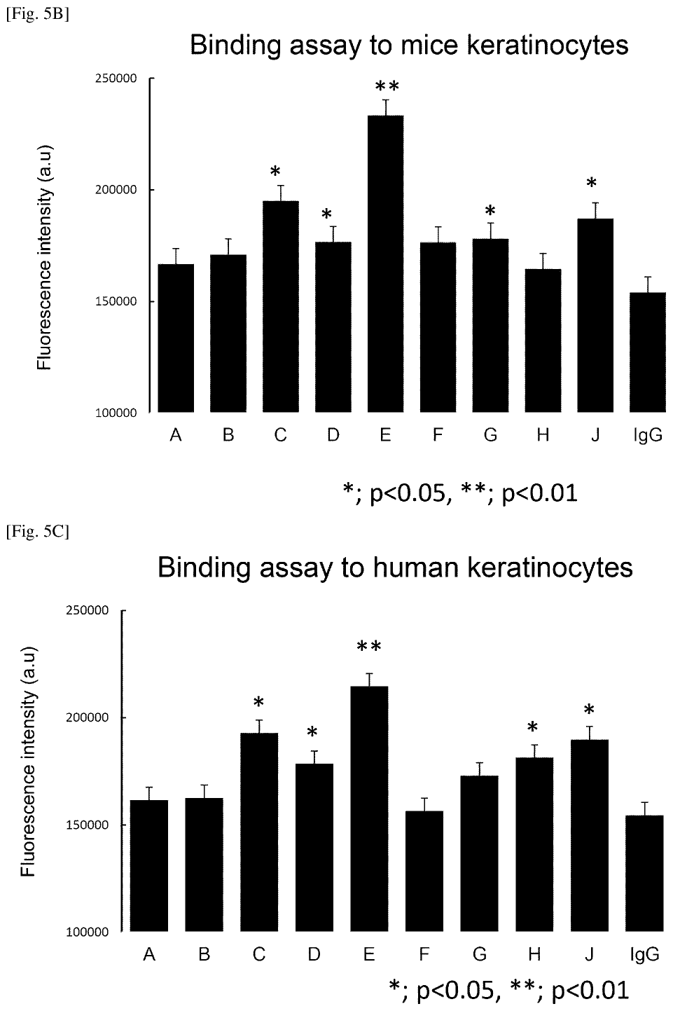

The result obtained by using PAM212 cells, which are mouse epithelial cells, and antibody J is shown in FIG. 5A.

The testing was also carried out using solvent (Veh) or a non-specific IgG (IgG) controls. In FIG. 5A, the vertical axis represents fluorescence intensity, and the mean fluorescence intensity of each sample is represented by bar height together with standard error. From all cases in which antibody J was used at any concentration of 0.1, 1, and 10 μg/mL, significantly increased fluorescence intensity was recognized compared to the controls (Veh and IgG) (in the drawing, ** represents the presence of a significant difference of P<0.01). It was found that antibody J specifically recognizes the mouse AQP3 on cell surface so that antibody J and PAM212 cells bind to each other.

The assay was also performed using PAM212 cells and antibodies A, B, C, D, E, F, G, H, and J at a concentration of 10 μg/mL. Results are shown in FIG. 5B. Binding of antibodies C, D, E, G, and J to PAM212 cells was statistically significant.

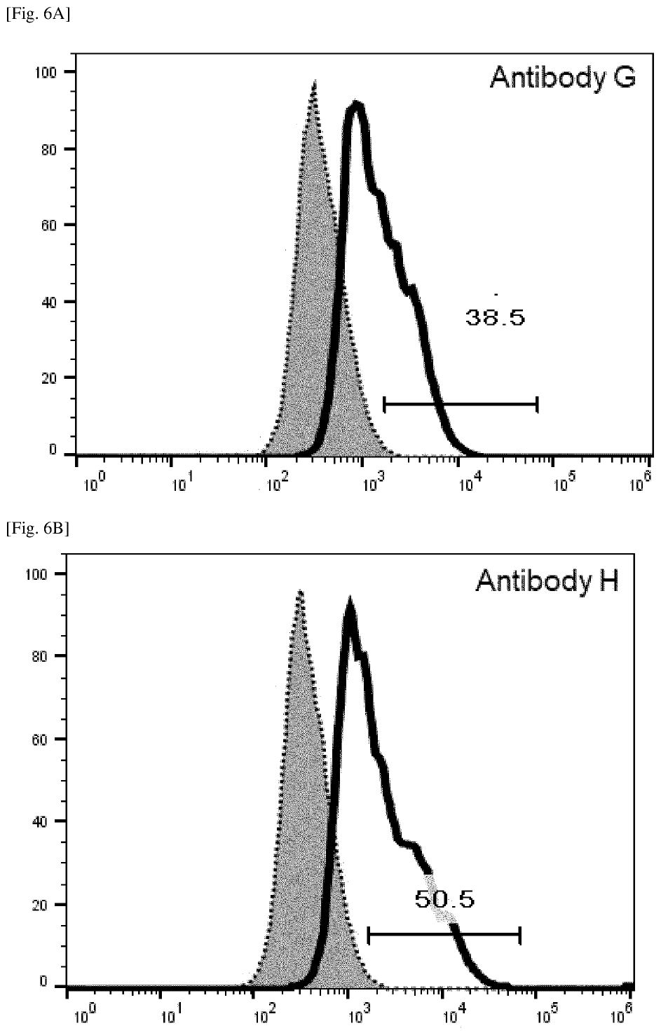

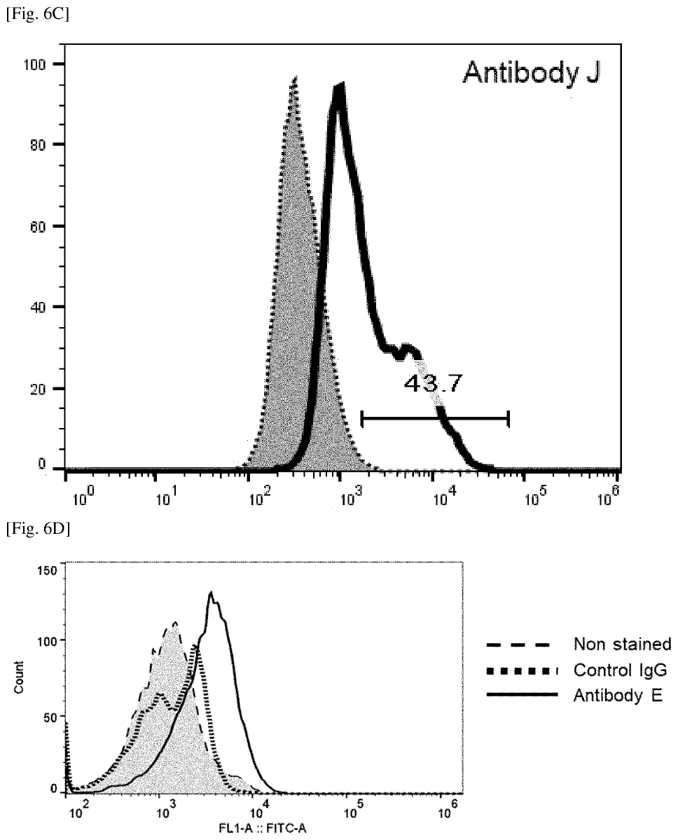

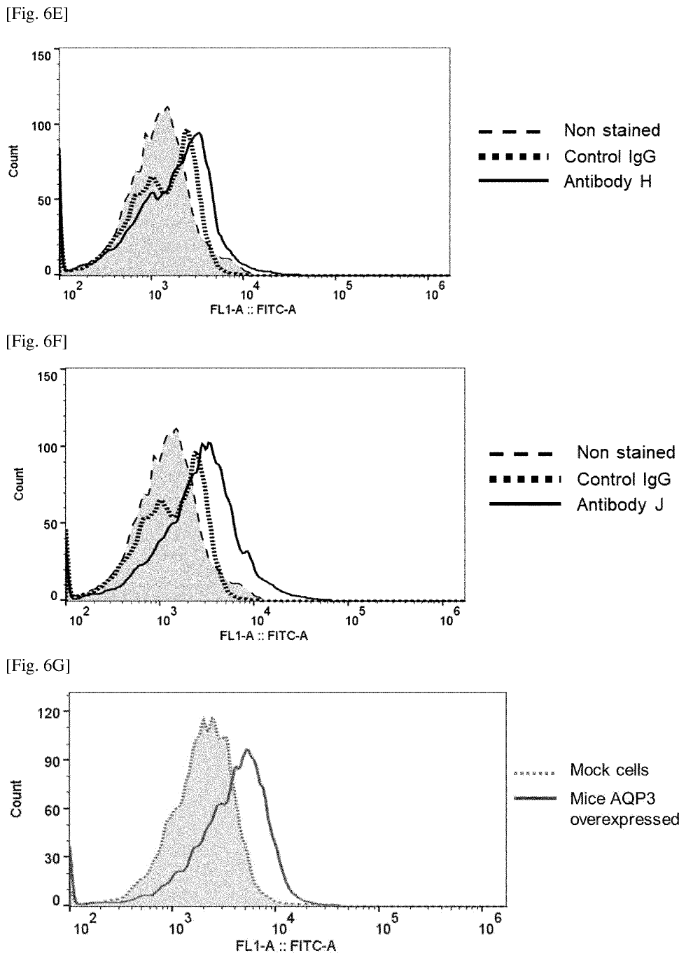

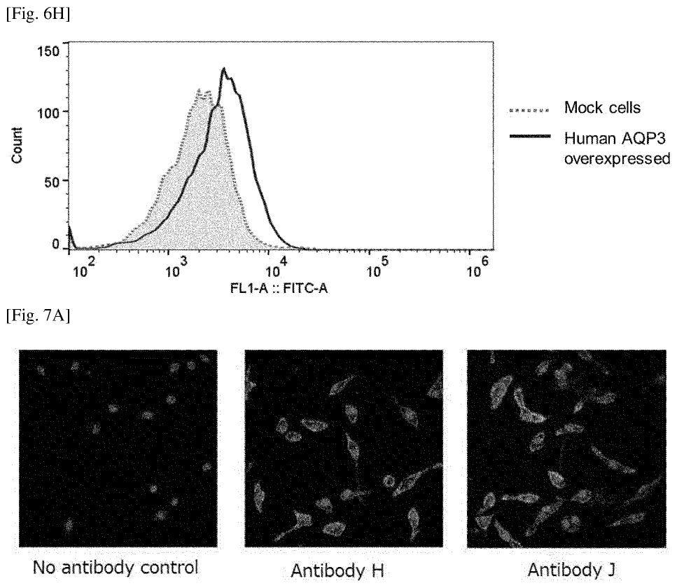

The assay was also performed using HaCaT cells and antibodies A, B, C, D, E, F, G, H, and J at a concentration of 10 μg/mL. Results are shown in FIG. 5C. Binding of antibodies C, D, E, H, and J to HaCaT cells was statistically significant. The results obtained by performing a FACS assay using HaCaT, which are human epithelial cells, and antibody G, antibody H, or antibody J are shown in FIG. 6A-6C, respectively.

HaCaT cells were treated with Cell Dissociation Buffer for 30 minutes at 37° C., and then dislodged and collected. Then, the cells were reacted with 10 μg/mL anti AQP3 antibody at 4° C. for 1 hour. After washing the cells, a fluorescent-labeled secondary antibody was added and the reaction was allowed to occur additionally for 1 hour (4° C.). Then, by using a flow cytometer, fluorescence intensity was measured (FIG. 6A to FIG. 6C). FIG. 6A represents the result of a case in which antibody G was used, FIG. 6B represents the result of a case in which antibody H was used, and FIG. 6C represents the result of a case in which antibody J was used. Each panel shows a histogram in which the horizontal axis represents fluorescence intensity and the vertical axis represents the cell number distribution when the mode value is set at 100. The histogram expressed with bold line represents a case in which the anti AQP3 antibody was used while the histogram expressed with thin dotted line represents a case as a control in which the anti AQP3 antibody was not used (addition of non-specific IgG). In the drawing, the range represented by a horizontal bar indicates the fluorescence intensity that is exhibited by the AQP3 antibody positive cell group. Ratio (%) of the cells included in this range (=cells showing positive staining by anti AQP3 antibody) is also shown in the drawing.

From all cases in which any of antibody G, antibody H, and antibody J was used, a clear increase in fluorescence intensity was recognized compared to the control, and thus it was found that the anti AQP3 antibodies have a binding activity for human AQP3 on cell surface.

A FACS assay was also performed using HEK293 cells stably overexpressing mouse AQP3. Cells were incubated with antibody E, H, J, or negative control IgG at a concentration of 10 μg/mL for one hour and then sorted by FACS. Separately, HEK293 cells stably overexpressing human AQP3 were incubated with antibody E at a concentration of 10 μg/mL for one hour and then sorted by FACS. The results are shown in FIGS. 6D-6H. Each of antibodies E, H, and J were found to bind to AQP3 over-expressed on surface of HEK293 cells.

From the above, several anti AQP3 antibodies were found to bind to the mouse macrophage cells, mouse epithelial cells (PAM212 cells), and human epithelial cells (HaCaT cells).

### Example 4

Immunostaining

By using mouse macrophage cells as AQP3-expressing cells, an immunohistochemistry analysis was made to see whether or not anti AQP3 antibodies can be used for immunostaining.

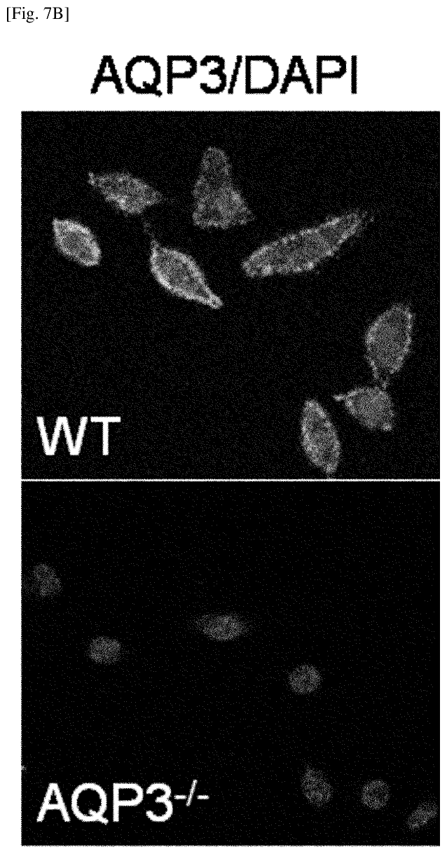

Blocking was carried out for a plate adhered with mouse macrophage cells, and then a reaction with 10 μg/mL anti AQP3 antibody was carried out for 1 hour at 4° C. After washing the cells, a fluorescent-labeled secondary antibody was added and the reaction was allowed to occur additionally for 1 hour (4° C.). As a control, a test not using the anti AQP3 antibody was also carried out. Furthermore, to have a clear location of cell nucleus, staining using DAPI was also carried out. Observation of the fluorescence staining was carried out by a confocal fluorescence microscope. The result obtained by using antibody H and antibody J is shown in FIG. 7A, together with the result of the control having no antibody. In FIG. 7A, the left panel shows an observation image of a case in which there was no antibody (anti AQP3 antibody was not present, only secondary antibody was present), the center panel shows an observation image of a case in which antibody H was used, and the right panel shows an observation image of a case in which antibody J was used. From all panels, a signal derived from DAPI with a dot-like shape showing the location of cell nucleus was recognized. Meanwhile, when antibody H or antibody J was used, a signal which appears to wrap around the edge of cell shape by enclosing the dot-like shape signal resulting from DAPI staining was also recognized. However, when the antibody was not present, a signal which appears to enclose the dot-like shape signal resulting from DAPI staining was not recognized at all.

Only a faint signal was observed when the immunostaining was performed using antibody J and mouse macrophage cells from AQP3 knock-out mice (Ma et al., 2000, PNAS, 97(8):4386-4391), showing that antibody J specifically binds to AQP3 expressing macrophage cells (FIG. 7B).

From the above, it was shown that the tested anti AQP3 antibodies are antibodies which can be used for an immunohistochemistry analysis.

### Example 5

Activity of Inhibiting Cell Proliferation

By using mouse epithelial cells (PAM212), mouse macrophage as mouse AQP3-expressing cells, human epithelial cells (HaCaT), or human epithelioid carcinoma cells (A431), the activity of inhibiting cell proliferation by an anti AQP3 antibody was measured.

Each of PAM212, HaCaT, and A431 were suspended in DMEM medium containing 1% FBS and seeded on a 96-well plate (5,000 cells/well). On the day after the seeding, DMEM medium containing anti AQP3 antibody (0.1, 1, or 10 μg/mL) was added and culture was continued for additional 2 days. The cell number was compared by using a reagent for measuring living cells (Nacalai Tesque Inc.) and measuring absorbance at 450 nm.

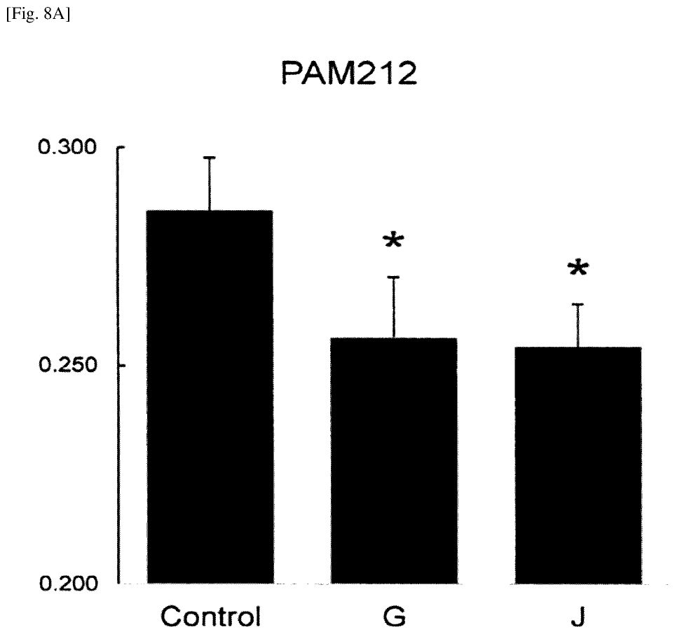

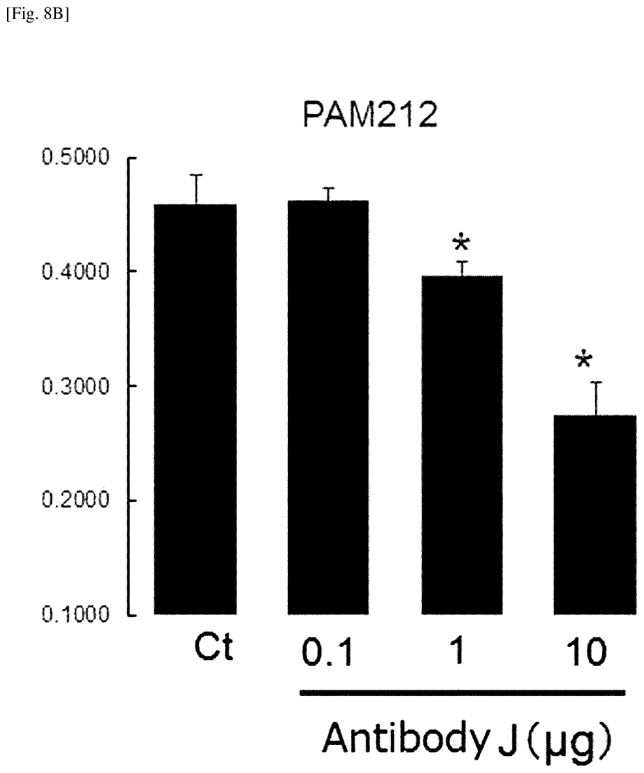

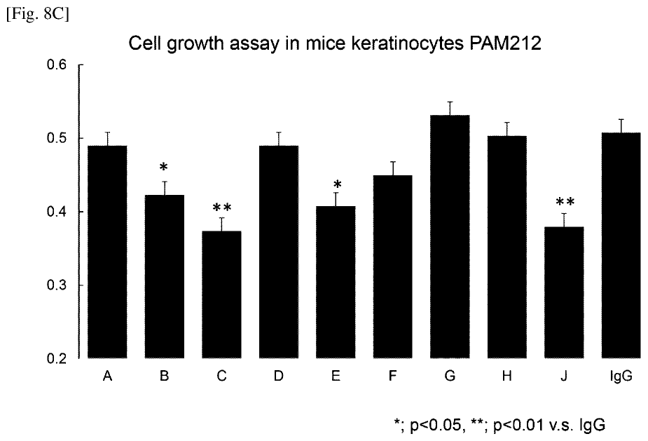

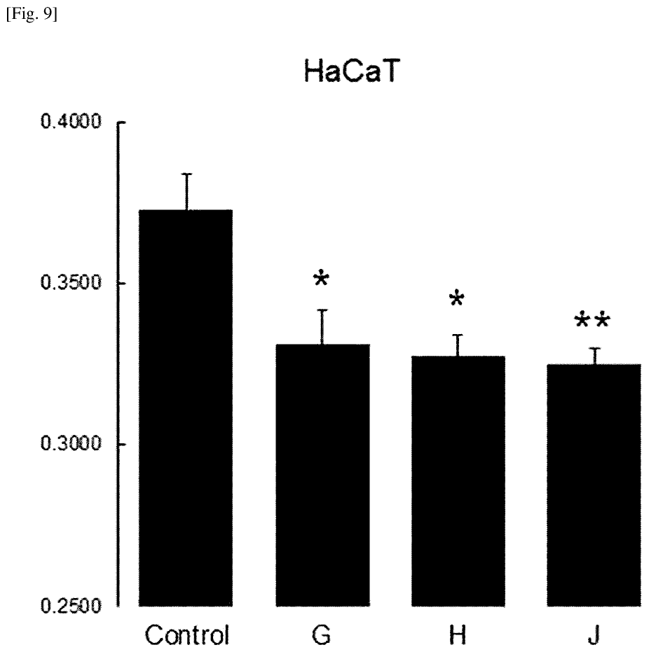

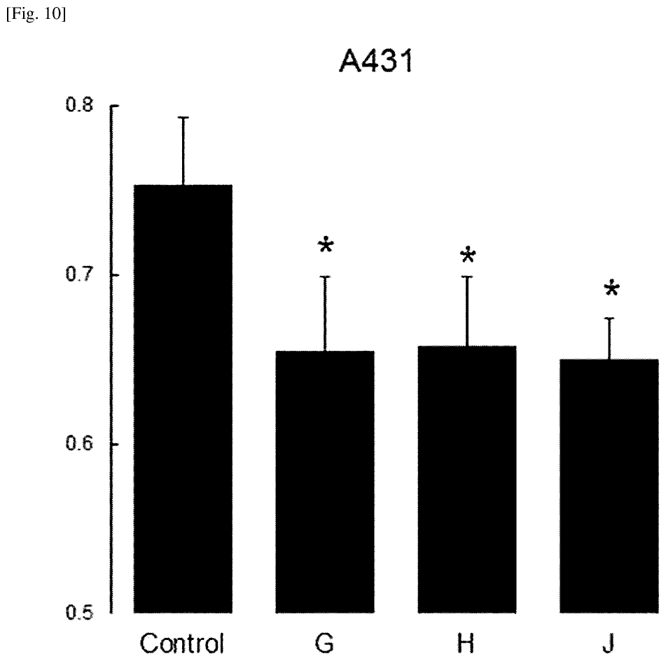

FIG. 8A and FIG. 8B represent a result obtained from a case in which PAM212 AQP3-expressing cells were used. The result obtained by using antibody G or antibody J is shown in FIG. 8A, together with the result of testing non-specific IgG as a control (non-specific IgG was added at 10 μg/mL; Control). In FIG. 8A, the vertical axis shows the absorbance at 450 nm, and the absorbance level was expressed by bar height, together with standard error (same for FIG. 8B, FIG. 9, and FIG. 10). The asterisk (*) in the drawing indicates that there is a significant difference of P<0.01 compared to the control. When antibody G or antibody J (10 μg/mL) was used, a significant inhibitory activity for PAM212 cell proliferation was observed.

Concentration-dependent effect of the anti AQP3 antibody J on the inhibitory activity for PAM212 cell proliferation was analyzed and is shown in FIG. 8B. Number of the living cells when antibody J was used at 0.1, 1, or 10 μg/mL is shown in FIG. 8B, together with the result of a non-specific IgG as a control (non-specific IgG was added at 10 μg/mL; Ct). The inhibitory activity for cell proliferation was increased by antibody J in a concentration-dependent manner. In FIG. 8B, the asterisk (*) described for the case in which in antibody J was used at 1 and 10 μg/mL indicates that there is a significant difference of P<0.01 compared to the control.

FIG. 8C. shows the effect of antibodies A, B, C, D, E, F, G, H, J, and a negative control IgG antibody at a concentration of 10 μg/mL on PAM212 cell growth. At the tested concentration, antibodies B, C, E, and J significantly inhibited cell growth.

FIG. 9 represents a result obtained from a case in which HaCaT cells were used as a material of human AQP3-expressing cells. The result obtained by using antibody G, antibody H, or antibody J is shown in FIG. 9, together with the result of testing a non-specific IgG as a control (non-specific IgG was added at 10 μg/mL; Control). In FIG. 9, the asterisks (*) and (**) indicate that there is a significant difference of P<0.05 or P<0.01, respectively, compared to the control. When antibody G, antibody H, or antibody J were used (10 μg/mL), a significant inhibitory activity for HaCaT cell proliferation was shown.

FIG. 10 represents a result obtained from a case in which A431 cells were used as a material of human AQP3-expressing cells. The result obtained by using antibody G, antibody H, or antibody J is shown in FIG. 10, together with the result of testing a non-specific IgG as a control (non-specific IgG was added at 10 μg/mL; Control). In FIG. 10, the asterisk (*) indicates that there is a significant difference of P<0.05 compared to the control. When antibody G, antibody H, or antibody J was used (10 μg/mL), a significant inhibitory activity for A431 cell proliferation was observed. Because A431 cells are a human squamous epithelial carcinoma cell line, the effect of inhibiting proliferation into AQP3-expressing cancer cells by the anti AQP3 antibodies was exhibited.

From the above, it was clearly shown that, at least with antibody G, antibody H, and antibody J, the significant inhibitory activity on the cell proliferation in AQP3-expressing cells including cancer cells is exhibited when co-culture of the anti AQP3 antibody and AQP3-expressing cells is carried out.

### Example 6

Activity of Inhibiting Hydrogen Peroxide Permeation

By using mouse macrophage as mouse AQP3-expressing cells, an activity of inhibiting the hydrogen peroxide permeation property (incorporating property) by an anti AQP3 antibody was measured.

Mouse macrophages were suspended in DMEM medium containing 1% FBS and seeded on a 96-well plate (10,000 cells/well). On the day after the seeding, DMEM medium containing antibody J (10 μg/mL) as an anti AQP3 antibody or 10 μg/mL control IgG antibody (Ct-IgG: IgG antibody not having specific binding property to AQP3) was added and co-culture was additionally continued overnight. To the culture, hydrogen peroxide (100 μM) or lipopolysaccharide (LPS) (300 ng/mL) was added, and the amount of reactive oxygen species (ROS) in the cells was measured. The ROS amount in the cells was evaluated by, after staining the cells by adding CM-H2DCFDA reagent (Invitrogen, 50 μM, for 20 minutes), measuring the fluorescence intensity derived from CM2DCF before and after the addition. If hydrogen peroxide as one kind of ROS permeates into the cell, it is possible to perform a measurement in which increased fluorescence intensity is taken as an indicator of an increased ROS amount in cells. Addition of LPS has a function of increasing artificially the ROS amount in cells.

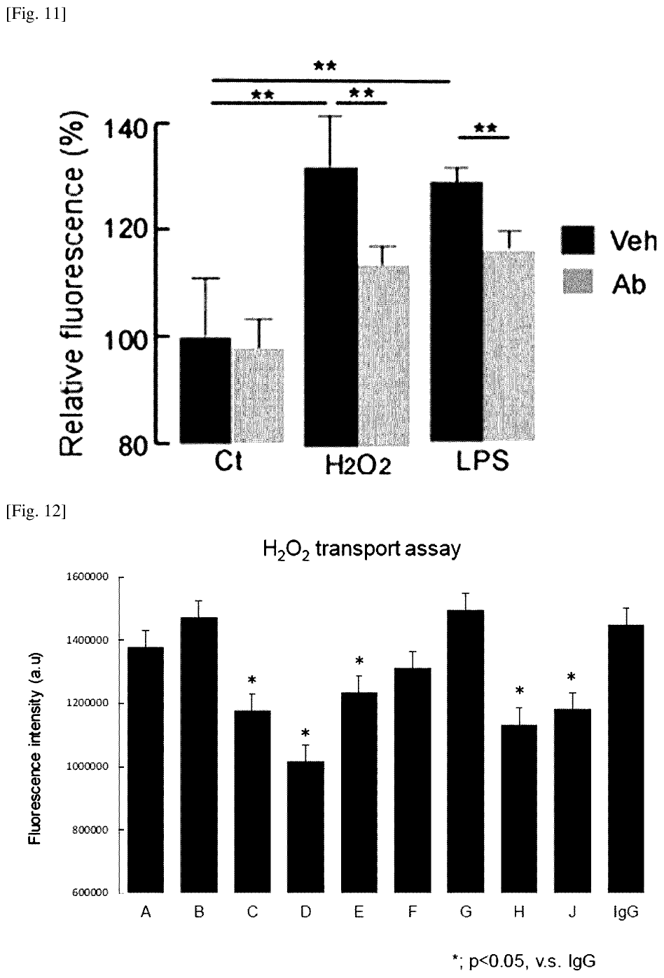

FIG. 11 shows the fluorescence intensity derived from CM2DCF when antibody J was added to a co-culture system (Ab) or a solvent was added to a co-culture system (Veh), for a case in which hydrogen peroxide was added (H₂O₂), a case in which lipopolysaccharide was added (LPS), or a case in which both H₂O₂ and LPS were not added (Ct) to the co-culture system. The vertical axis represents a relative value of the fluorescence intensity. A case of applying a solvent to the cells which have been added with Ct-IgG antibody (left bars in the drawing) is set at 100%, and the relative fluorescence intensity at each condition is represented by bar height, together with standard error. In the drawing, the asterisk (**) indicates that there is a significant difference of P<0.01 among the comparisons, and it is clearly shown that, when hydrogen peroxide was added or LPS was added, the ROS amount in cells significantly increased compared to Veh group added with a solvent, and, at any conditions of adding hydrogen peroxide or adding LPS, if antibody J was present during the co-culture, the ROS amount in cells significantly decreased compared to a case in which antibody J was absent.

FIG. 12 shows the results of an H₂O₂ transport assay performed using antibodies A, B, C, D, E, F, G, H, and J.

Antibodies C, D, E, H, and J have an activity of significantly suppressing the incor-poration of hydrogen peroxide to the inside of AQP3-expressing cells.

### Example 7

Cell Signal Inhibitory Activity

It is known that, in mouse macrophage, p65/NFκB is phosphorylated and activated in accordance with the stimulation by LPS. To determine whether or not the cell signal responding to LPS is inhibited by an anti AQP3 antibody in mouse macrophage, which is a mouse AQP3-expressing cell, a test was carried out.

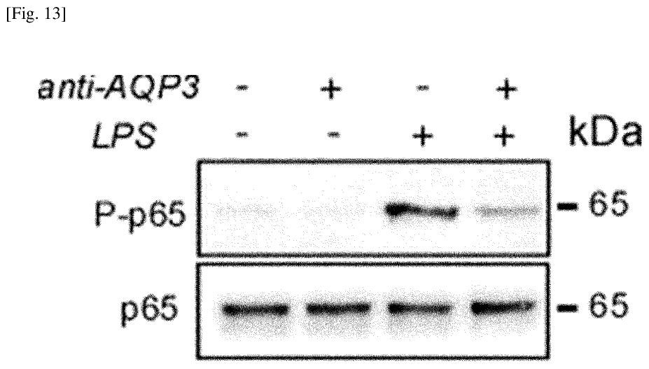

Mouse macrophages were suspended in DMEM medium containing 1% FBS and seeded on a 60 mm dish (2×10⁶ cells/dish). On the day after the seeding, DMEM medium containing antibody J (10 μg/mL) as an anti AQP3 antibody or 10 μg/mL control IgG antibody (non-specific IgG antibody) was added and co-culture was additionally continued overnight (in FIG. 13 showing the result, the former condition was described as “anti-AQP3+”, while the latter condition was described as “anti-AQP3 −”). Each cultured product under both conditions was subjected to a treatment with LPS (100 ng/mL, for 1 hour) or a no treatment with LPS (in FIG. 13 showing the result, the former condition was described as “LPS+”, while the latter condition was described as “LPS −”). According to the addition/no addition of the anti AQP3 antibody and the treatment/no treatment with LPS, four treatment groups were created with the mouse macrophage as a sample. From each cell of the four treatment groups, proteins were extracted, and phosphorylation state of p65/NFκB was determined by immunoblotting for each group.

FIG. 13 shows the result of carrying out immunoblotting by using an antibody which is specific to each of non-phosphorylated p65 (p65) and phosphorylated p65 (P-p65).

While phosphorylated p65 was strongly induced by LPS treatment at the condition of “anti-AQP3 −” (compare the top panel signals of the left most column with the second column from the right side), at the condition “anti-AQP3+” in which an anti AQP3 antibody was present, induction of phosphorylated p65 (P-p65) by LPS treatment was inhibited (compare the top panel signals of the second column from the left side with the right most column, and, for comparison between conditions regarding LPS addition, compare the top panel signals of the two right columns).

For the intracellular signal in which LPS-induced p65/NFκB is involved with the phosphorylation and activation in AQP3-expressing cells, antibody J has an inhibitory activity.

### Example 8

Inhibitory Activity on Liver Disorder (Acute Hepatitis and Acute Liver Disorder)

A test was carried out to determine in an animal subject the anti-inflammatory activity of an anti AQP3 antibody (inflammation inhibiting activity and disorder inhibiting activity).

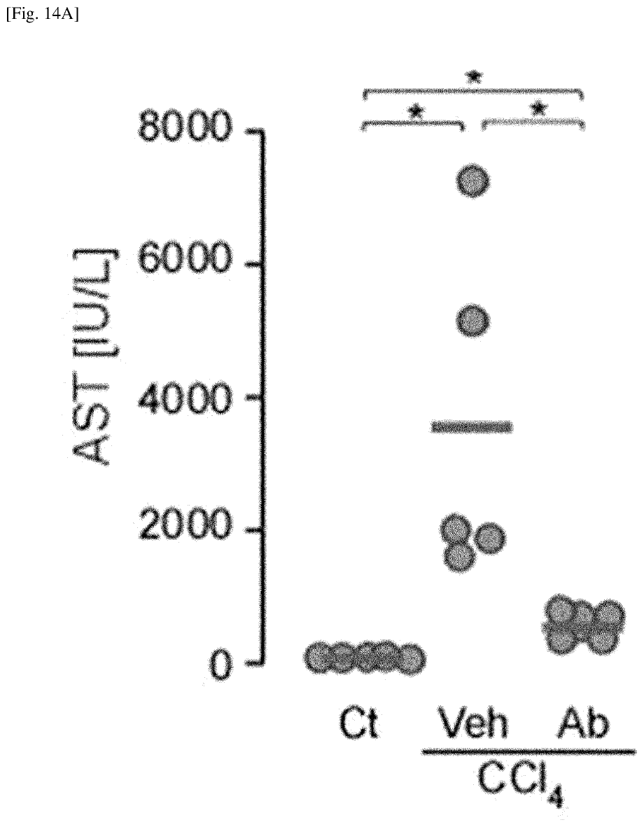

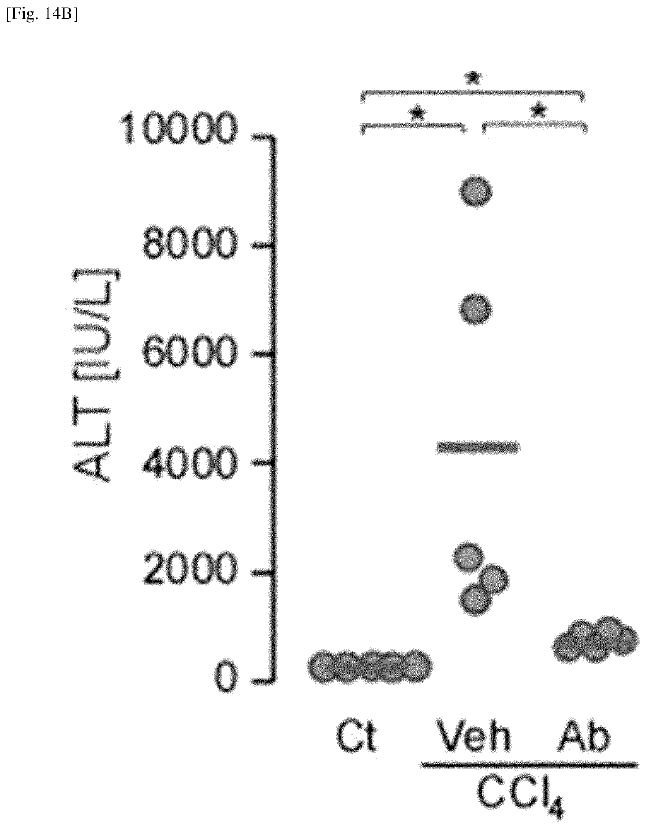

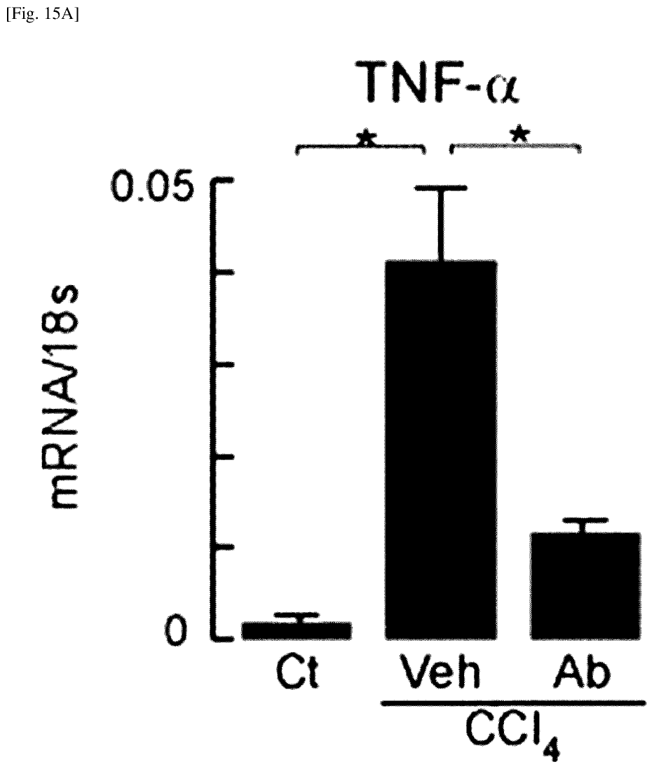

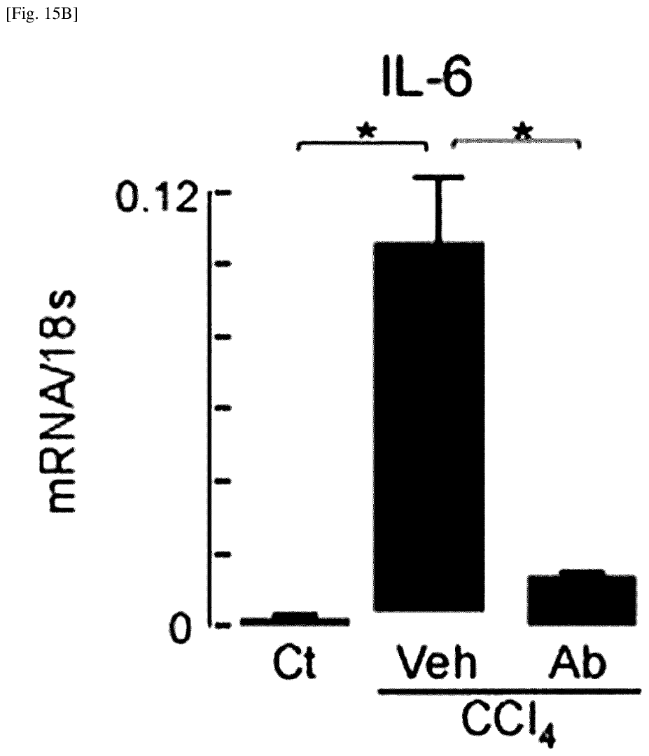

A mouse was used as a test material. The mouse was administered intravenously with an anti AQP3 antibody (antibody J) (5 μg/g of body weight). On the day after the administration, carbon tetrachloride (CCl4), which is a chemical for inducing a liver disorder (acute hepatitis and acute liver disorder), was administered (0.5 μ/g of body weight). 24 Hours after administering the carbon tetrachloride, blood serum and a liver RNA sample were collected. Blood serum AST value, blood serum ALT value, accumulation level of liver TNF-α mRNA, and accumulation level of liver IL-6 mRNA, as an indicator of the degree of the liver disorder, were evaluated. The analysis results using the blood sample and the analysis using the liver RNA sample are shown in FIG. 14 and FIG. 15, respectively.

FIG. 14A shows the analysis result of blood serum AST level. In the drawing, the vertical axis represents the AST level [IU/L], each spot represented by “◯” shows an individual measurement value, and the horizontal bar indicates a median value. Ct means a control that has not been subjected to a treatment with carbon tetrachloride. In the carbon tetrachloride treatment group (CCl4), Ab represents a group which has been treated in advance with an anti AQP3 antibody (antibody J) and Veh represents a group which has not been treated with an anti AQP3 antibody. In the drawing, the asterisk (*) indicates that there is a significant difference of p<0.01 between the carbon tetrachloride treatment group (both of Veh group and Ab group) and the control group (Ct), and also there is a significant difference of p<0.01 between Veh group and Ab group within the carbon tetrachloride treatment group.

FIG. 14B shows the analysis result of blood serum ALT level. In the drawing, the vertical axis represents the ALT level [IU/L], each spot represented by “◯” shows an individual measurement value, and the horizontal bar indicates a median value. Ct means a control that has not been subjected to a treatment with carbon tetrachloride. In the carbon tetrachloride treatment group (CCl4), Ab represents a group which has been treated in advance with an anti AQP3 antibody (antibody J) and Veh represents a group which has not been treated with an anti AQP3 antibody. In the drawing, the asterisk (*) indicates that there is a significant difference of p<0.01 between the carbon tetrachloride treatment group (both of Veh group and Ab group) and the control group (Ct), and also there is a significant difference of p<0.01 between Veh group and Ab group within the carbon tetrachloride treatment group.

It is widely known that both the blood serum AST value and blood serum ALT value can be an indicator of a liver disorder (acute hepatitis and acute liver disorder). From the above test results, it is understood that, in a mouse which has been treated in advance with an anti AQP3 antibody, a liver disorder and/or liver inflammation reaction that is caused later by carbon tetrachloride can be prevented or inhibited.

FIG. 15A shows the analysis result of accumulation level of TNF-α mRNA in a liver homogenates. In the drawing, the vertical axis represents the TNF-α expression level, which was obtained by dividing the accumulation level of TNF-α mRNA by 18s rRNA level as a control. In the drawing, TNF-α expression level is shown by bar height together with standard error. Ct means a control that has not been subjected to a treatment with carbon tetrachloride. In the carbon tetrachloride treatment group (CCl4), Ab represents a group which has been treated in advance with an anti AQP3 antibody (antibody J) and Veh represents a group which has not been treated with an anti AQP3 antibody. In the drawing, the asterisk (*) indicates that there is a significant difference of p<0.01 between Veh group and the control group (Ct), and also between Veh group and Ab group.

FIG. 15B shows the analysis result of accumulation level of IL-6 mRNA in a liver homogenates. In the drawing, the vertical axis represents the IL-6 expression level, which was obtained by dividing the accumulation level of IL-6 mRNA by 18s rRNA level as a control. In the drawing, IL-6 expression level is shown by bar height together with standard error. Ct means a control that has not been subjected to a treatment with carbon tetrachloride. In the carbon tetrachloride treatment group (CCl4), Ab represents a group which has been treated in advance with an anti AQP3 antibody (antibody J) and Veh represents a group which has not been treated with an anti AQP3 antibody. In the drawing, the asterisk (*) indicates that there is a significant difference of p<0.01 between Veh group and the control group (Ct), and also between Veh group and Ab group.

It is widely known that expression of TNF-α or IL-6 in liver is an indicator of a liver disorder (acute hepatitis and acute liver disorder). From the above test results, it is understood that, in a mouse which has been treated in advance with an anti AQP3 antibody, a liver disorder and/or liver inflammation reaction that is caused later by carbon tetrachloride can be prevented or inhibited.

For a case of an individual animal which may have a liver disorder (acute hepatitis and acute liver disorder), an occurrence of liver disorder or inflammatory response can be prevented or inhibited by an anti AQP3 antibody.

### Example 9

Sequence Analysis of Anti AQP3 Antibodies

The amino acid sequence of the heavy chain and light chain was determined for each of antibodies A, B, C, D, E, F, G, H, J, and K. The heavy chain and light chain sequences (without the predicted signal sequences, which are the same for antibodies A, B, C, D, E, F, G, H, J, and K) are shown in Table 1.

TABLE 1
Heavy Chain (HC) and Light Chain (LC) Sequences

| | | SEQ |
|---|---|---|
| Description | Sequence | ID NO: |
| Antibody A | QVQLQQPGAELVRPGASVTLSCKASGYTFTDYEMHWVKQTPV | 123 |
| HC | HGLEWIGGVDPETGGTGYNQKFRGKAILTADKSSSTAYMELR | |
| | SLTSEDSAVYYCARHGGSFYAMDYWGQGTSVTVSSAKTTAPS | |
| | VYPLAPVCGDTTGSSVTLGCLVKGYFPEPVTLTWNSGSLSSG | |
| | VHTFPAVLQSDLYTLSSSVTVTSSTWPSQSITCNVAHPASST | |
| | KVDKKIEPRGPTIKPCPPCKCPAPNLLGGPSVFIFPPKIKDV | |
| | LMISLSPIVTCVVVDVSEDDPDVQISWFVNNEVEVHTQTQTH | |
| | REDYNSTLRVVSALPIQHQDWMSGKEFKCKVNNKDLPAPIER | |
| | TISKPKGSVRAPQVYVLPPPEEEMTKKQVTLTCMVTDFMPED | |
| | IYVEWTNNGKTELNYKNTEPVLDSDGSYFMYSKLRVEKKNWV | |
| | ERNSYSCSVVHEGLHNHHTTKSFSRTPGK | |
| Antibody A | DIVMTQSPKFMSTSVGDRVSITCKASQDVSTAVAWYQQKPGQ | 124 |
| LC | SPKLLIYWASTRHTGVPDRFTGSGSGTDYTLTISSVQAEDLA | |
| | LYYCQQHYSTPPTFGGGTKLELKRADAAPTVSIFPPSSEQLT | |
| | SGGASVVCFLNNFYPDKINVKWKIDGSERQNGVLNSWTDQDS | |
| | KDSTYSMSSTLTLTKDEYERHNSYTCEATHKTSTSPIVKSFN | |
| | RNEC | |
| Antibody B | EVQLVESGGDLVKPGGSLKLSCAASGFTFSSYGMSWVRQTPD | 125 |
| HC | KRLEWVATISRGSIYTYYPDSVKGRFTISRDNAKNTLYLQMS | |
| | SLKSEDTAMYYCARLSLYDYDGARYTMDYWGQGTSVTVSSAK | |
| | TTAPSVYPLAPVCGDTTGSSVTLGCLVKGYFPEPVTLTWNSG | |
| | SLSSGVHTFPAVLQSDLYTLSSSVTVTSSTWPSQSITCNVAH | |
| | PASSTKVDKKIEPRGPTIKPCPPCKCPAPNLLGGPSVFIFPP | |
| | KIKDVLMISLSPIVTCVVVDVSEDDPDVQISWFVNNVEVHTA | |
| | QTQTHREDYNSTLRVVSALPIQHQDWMSGKEFKCKVNNKDLP | |
| | APIERTISKPKGSVRAPQVYVLPPPEEEMTKKQVTLTCMVTD | |
| | FMPEDIYVEWTNNGKTELNYKNTEPVLDSDGSYFMYSKLRVE | |
| | KKNWVERNSYSCSVVHEGLHNHHTTKSFSRTPGK | |
| Antibody B | DIVMTQSPKFMSTSVGDRVSITCKASQDVGTAVAWYQQKPGQ | 126 |
| LC | SPKLLIYWASTRHTGVPDRFTGSGSGTDFTLTISNVQSEDLA | |
| | DYFCQQYSSYHTFGAGTKLELKRADAAPTVSIFPPSSEQLTS | |
| | GGASVVCFLNNFYPKDINVKWKIDGSERQNGVLNSWTDQDSK | |
| | DSTYSMSSTLTLTKDEYERHNSYTCEATHKTSTSPIVKSFNR | |
| | NEC | |
| Antibody C | QVQLKQSGAELARPGASVKLSCKASGYNFKSYGISWVKQRTG | 127 |
| HC | QGLEWIGEIYPGSGNTYYNEKLKGKATLTADKSSSTAYMELR | |
| | SLTSEDSAVYFCARTYGYDSFPWFAYWGQGTLVTVSSAKTTA | |
| | PSVYPLAPVCGDTTGSSVTLGCLVKGYFPEPVTLTWNSGSLS | |
| | SGVHTFPAVLQSDLYTLSSSVTVTSSTWPSQSITCNVAHPAS | |

TABLE 2
Table 1
Heavy Chain (HC) and Light Chain (LC) Sequences

| | | SEQ |
|---|---|---|
| Description | Sequence | ID NO: |
| | STKVDKKIEPRGPTIKPCPPCKCPAPNLLGGPSVFIFPPKIK | |
| | DVLMISLSPIVTCVVVDVSEDDPDVQISWFVNNVEVHTAQTQ | |
| | THREDYNSTLRVVSALPIQHQDWMSGKEENCKVNNKDLPAPI | |
| | ERTISKPKGSVRAPQVYVLPPPEEEMTKKQVTLTCMVTDEMP | |
| | EDIYVEWTNNGKTELNYKNTEPVLDSDGSYFMYSKLRVEKKN | |
| | WVERNSYSCSVVHEGLHNHHTTKSFSRTPGK | |
| Antibody C | DIVMTQAAPSVPVTPGESVSISCRSSKSLLHSNGNTYLYWFL | 128 |
| LC | QRPGQSPQLLIYRVSNLASGVPDRFSGSGSGTAFTLRISRVE | |
| | AEDEGVYYCMQHLEYPFTFGAGTKLEIKRADAAPTVSIFFTS | |
| | SEQLTSGGASVVCFLNNFYPKDINVKWKIDGSERQNGVLNSW | |
| | TDQDSKDSTYSMSSTLTLTKDEYERHNSYTCEATHKTSTSPI | |
| | VKSFNRNEC | |
| Antibody D | EVQLQQSGAELVRPGASVTLSCKASGYTFTDYEMHWVQQTPV | 129 |
| HC | HGLEWIGGIDPETGGTGYNQKFKGKAILTADKSSSTAYMELR | |
| | SLTSEDSAVYFCTRHGSYAMDYWGQGTSVTVSSAKTTAPSVY | |
| | PLAPVCGDTTGSSVTLGCLVKGYFPEPVTLTWNSGSLSSGVH | |
| | TFPAVLQSDLYTLSSSVTVTSSTWPSQSITCNVAHPASSTKV | |
| | DKKIEPRGPTIKPCPPCKCPAPNLLGGPSVFIFPPKIKDVLM | |
| | ISLSPIVTCVVVDVSEDDPDVQISWFVNNVEVHTAQTQTHRE | |
| | DYNSTLRVVSALPIQHQDWMSGKEFKCKVNNKDLPAPIERTI | |
| | SKPKGSVRAPQVYVLPPPEEEMTKKQVTLTCMVTDEMPEDIY | |
| | VEWTNNGKTELNYKNTEPVLDSDGSYFMYSKLRVEKKNWVER | |
| | NSYSCSVVHEGLHNHHTTKSFSRTPGK | |
| Antibody D | DIVMTQSPKFMSTSVGDRVSITCKASQDVSTAVAWYQQKPGQ | 130 |
| LC | SPKLLIYWASTRHTGVPDRFTGSGSGTDYTLTISSVQAEDLA | |
| | LYYCQQHYSTPPTFGGGTRLEIKRADAAPTVSIFPPSSEQLT | |
| | SGGASVVCFLNNFYPKDINVKWKIDGSERQNGVLNSWTDQDS | |
| | KDSTYSMSSTLTLTKDEYERHNSYTCEATHKTSTSPIVKSFN | |
| | RNEC | |
| Antibody E | EVKLLESGAELVRPGASVTLSCKASGYTFTDYEMHWVKQTPV | 131 |
| HC | HGLEWIGGIDPESGGTGYNQKFKGKAILTADKSSSTAYMELR | |
| | SLTSEDSAVYFCTRSGYYGSPLLDYWGQGTTLTVSSAKTTAP | |
| | SVYPLAPVCGDTTGSSVTLGCLVKGYFPEPVTLTWNSGSLSS | |
| | GVHTFPAVLQSDLYTLSSSVTVTSSTWPSQSITCNVAHPASS | |
| | TKVDKKIEPRGPTIKPCPPCKCPAPNLLGGPSVFIFPPKIKD | |
| | VLMISLSPIVTCVVVDVSEDDPDVQISWFVNNVEVHTAQTQT | |
| | HREDYNSTLRVVSALPIQHQDWMSGKEFKCKVNNKDLPAPIE | |
| | RTISKPKGSVRAPQVYVLPPPEEEMTKKQVTLTCMVTDFMPE | |
| | DIYVEWTNNGKTELNYKNTEPVLDSDGSYFMYSKLRVEKKNW | |
| | VERNSYSCSVVHEGLHNHHTTKSFSRTPGK | |

TABLE 3
Table 1
Heavy Chain (HC) and Light Chain (LC) Sequences

| | | SEQ |
|---|---|---|
| Description | Sequence | ID NO: |
| Antiboy E | QIVLSQSPAIMSASLGERVTMTCTASSSVSSSYLHWYQQKPG | 132 |
| LC | SSPKLWIYSTSNLASGVPARFSGSGSGTSYSLTISSMEAEDA | |
| | ATYYCHQYHRSPPTFGAGTKLEIKRADAAPTVSIFPPSSEQL | |
| | TSGGASVVCFLNNFYPKDINVKWKIDGSERQNGVLNSWTDQD | |
| | SKDSTYSMSSTLTLTKDEYERHNSYTCEATHKTSTSPIVKSF | |
| | NREC | |
| Antiboy F | QVQLKESGPELVKPGASVKISCKASGYTFTDYYINWVKQRPG | 133 |
| HC | QGLEWIGWIFPGSGSTYYNEKFKGKATLTVDKSSSTAYMLLS | |
| | SLTSEDSAVYFCADYGSSYRYFDVWGAGTTVTVSSAKTTAPS | |
| | VYPLAPVCGDTTGSSVTLGCLVKGYFPEPVTLTWNSGSLSSG | |
| | VHTFPAVLQSDLYTLSSSVTVTSSTWPSQSITCNVAHPASST | |
| | KVDKKIEPRGPTIKPCPPCKCPAPNLLGGPSVFIFPPKIKDV | |
| | LMISLSPIVTCVVVDVSEDDPDVQTSWFVNNVEVHTAQTQTH | |
| | REDYNSTLRVVSALPIQHQDWMSGKEFKCKVNNKDLPAPIER | |
| | TISKPKGSVRAPQVYVLPPPEEEMTKKQVTLTCMVTDFMPED | |
| | IYVEWTNNGKTELNYKNTEPVLDSDGSYFMYSKLRVEKKNWV | |
| | ERNSYSCSVVHEGLHNHHTTKSFSRTPGK | |
| Antiboy F | DIVMTQSPAILSASPGEKVTMTCRASSSVSYMHWYQQKPGSS | 134 |
| LC | PKPWIYATSYLASGVPARFSGSGSGTSYSLTIGRVEAEDAAT | |
| | YYCQQWSSNPLTFGAGTKLELKRADAAPTVSIFPPSSEQLTS | |
| | GGASVVCFLNNFYPKDINVKWKIDGSERQNGVLNSWTDQDSK | |
| | DSTYSMSSTLTLTKDEYERHNSYTCEATHKTSTSPIVKSFNR | |
| | NEC | |
| Antiboy G | QVQLKQSGAELVRPGASVTLSCKASGYTFTDYFMHWVKQTPV | 135 |
| HC | HGLEWIGGIDPETGGTAYNQKFKGKAILTADKSSSTAYMELR | |
| | SLTSEDSAVYYCTRWGAITSFVALRGFAYWGQGTLVTVSSAK | |
| | TTAPSVYPLAPVCGDTTGSSVTLGCLVKGYFPEPVTLTWNSG | |
| | SLSSGVHTFPAVLQSDLYTLSSSVTVTSSTWPSQSITCNVAH | |
| | PASSTKVDKKIEPRGDTIKPCPPCKCPAPNLLGGPSVFIFPP | |
| | KIKDVLMISLSPIVTCVVVDVSEDDPDVQISWFVNNVEVHTA | |
| | QTQTHREDYNSTLRVVSALPIQHQDWMSGKEFKCKVNNKDLP | |
| | APIERTISKPKGSVRAPQVYVLPPPEEEMTKKQVTLTCMVTD | |
| | FMPEDIYVEWTNNGKTELNYKNTEPVLDSDGSYFMYSKLRVE | |
| | KKNWVERNSYSCSVVHEGLHNHHTTKSFSRTPGK | |
| Antiboy G | DIQMTQSPSSLSVSAGEKVTMSCKSSQSLLNSGNQKNYLAWY | 136 |
| LC | QQKPGQPPKLLIYGASTRESGVPDRFTGSGSGTDFTLTISSV | |
| | QAEDLAVYYCQNDHSYPPTFGAGTKLELKRADAAPTVSIFPP | |
| | SSEQLTSGGASVVCFLNNFYPKDINVKWKIDGSERQNGVLNS | |
| | WTDQDSKDSTYSMSSTLTLTKDEYERHNSYTCATHKTSTSP | |
| | IVKSFNRNEC | |

TABLE 4
Table 1
Heavy Chain (HC) and Light Chain (LC) Sequences

| | | SEQ |
|---|---|---|
| Description | Sequence | ID NO: |
| Antibody H | EVKLVESGGDLVKPGGSLKLSCAASGFTFSSYGMSWVRQPTD | 137 |
| HC | KRLEWVATISRRSIYTYYPDSVQGRFTISRDNAKNTLYLQMS | |
| | SLKSEDTAMYYCARLSLYDYDGARYTMDYWGQGTSVTVSSAK | |
| | TTAPSVYPLAPVCGDTTGSSVTLGCLVKGYFPEPVTLTWNSG | |
| | SLSSGVHTFPAVLQSDLYTLSSSVTVTSSTWPSQSITCNVAH | |
| | PASSTKVDKKIEPRGPTIKPCPPCKCPAPNLLGGPSVFIFPP | |
| | KIKDMVLISLSPIVTCVVVDVSEDDPDVQISWFVNNVEVHTA | |
| | QTQTHREDYNSTLRVVSALPIQHQDWMSGKEFKCKVNNKDLP | |
| | APIERTISKPKGSVRAPQVYVLPPPEEEMTKKQVTLTCMVTD | |
| | FMPEDIYVEWTNNGKTELNYKNTEPVLDSDGSYFMYSKLRVE | |
| | KKNWVERNSYSCSVVHEGLHNHHTTKSFSRTPGK | |
| Antibody H | KIKMTQSPKFMSTSVGDRVSITCKASQDVGTAVAWYQQKPGQ | 138 |
| LC | SPKLLIYWASTRHTGVPDRFTGSGSGTDFTLTISNVQSEDLA | |
| | DYFCQQYSSYHTFGAGTKLEIKRADAAPTVSIFPPSSEQLTS | |
| | GGASVVCFLNNFYPKDINVKWKIDGSERQNGVLNSWTDQDSK | |
| | DSTYSMSSTLTLTKDEYERHNSYTCEATHKTSTSPIVKSFKR | |
| | NEC | |
| Antibody J | QVHLQQSGTELVKPGASVKLSCEASGYTFTSYWMHWVKQRPG | 139 |
| HC | QGLEWIGNINPSNGGTNYNEKFKSKATLTVDKSSSTAYMQLS | |
| | SLTSEDSAVYYCARGGIYYGNYDYYAMDYWGQGTSVTVSSAK | |
| | TTAPSVYPLAPVCGDTTGSSVTLGCLVKGYFPEPVTLTWNSG | |
| | SLSSGVHTFPAVLQSDLYTLSSSVTVTSSTWPSQSITCNVAH | |
| | PASSTKVDKKIEPRGPTIKPCPPCKCPAPNLLGGPSVFIFPP | |
| | KIKDVLMISLSPIVTCVVVDVSEDDPDVQISWFVNNVEVHTA | |
| | QTQTHREDYNSTLRVVSALPIQHQDWMSGKEFKCKVNNKDLP | |
| | APIERTISKPKGSVRAPQVYVLPPPEEEMTKKQVTLTCMVTD | |
| | FMPEDIYVEWTNNGKTELNYKNTEPVLDSDGSYFMYSKLRVE | |
| | KKNWVERNSYSCSVVHEGLHNHHTTKSFSRTPGK | |
| Antibody J | DIVMTQAAPSVPVTPGESVSISCRSSKSLLHSNGNTYLYWFL | 140 |
| LC | QRPGQSPQLLIYRVSNLASGVPDRFSGSGSGTAFTLRISRVE | |
| | AEDVGVYYCMQHLEYPFTFGGGTKLEIKRADAAPTVSIFPPS | |
| | SEQLTSGGASVVCFLNNFYPKDINVKWKIDGSERQNGVLNSW | |
| | TDQDSKDSTYSMSSTLTLTKDEYERHNSYTCEATHKTSTSPI | |
| | VKSFNRNEC | |
| Antibody K | QVQLKQSGAELVRPGTSVKVSCKASGYAFTNYLIEWVKQRPG | 141 |
| HC | QGLEWIGVINPGSGGTNYNEKFKGKATLTADKSSSTAYMQLS | |
| | SLTSEDSAVYFCARWGFYYAMDYWGQGTSVTVSSAKTTAPSV | |
| | YPLAPVCGDTTGSSVTLGCLVKGYFPEPVTLTWNSGSLSSGV | |
| | HTFPAVLQSDLYTLSSSVTVTSSTWPSQSITCNVAHPASSTK | |
| | VDKKIEPRGPTIKPCPPCKCPAPNLLGGPSVFIFPPKIKDVL | |
| | MISLSPIVTCVVVDVSEDDPDVQISWFVNNVEVHTAQTQTHR | |

TABLE 5
Table 1
Heavy Chain (HC) and Light Chain (LC) Sequences

| | | SEQ |
|---|---|---|
| Description | Sequence | ID NO: |
| | EDYNSTLEVVSALPIQHQDWMSGKEFKCKVNNKDLPAPIERT | |
| | ISKPKGSVRAPQVYVLPETEEEMTKKQVILTCMVTDFMPEDI | |
| | YVEWTNNGKTELNYKNTEPVLDSDGSYFMYSKLEVEKKNWVE | |
| | RNSYSCSVVHEGLHNHHTTKSFSRTPGK | |
| Antibody K | DIVMTQSPSSLSASLGERVSLTCRASQEISGYLSWLQQKPDG | 142 |
| LC | TIKRLIYAASTLDSGVPKRESGSRSGSDYSLTISSLESEDFA | |
| | DYYCLQYASYPLTFGAGTKLEIKRADAAPTVSIFPPSSEQLT | |
| | SGGASVVCFLNNEYPKDINVKWKIDGSERQNGVLNSWTDQDS | |
| | KDSTYSMSSTLTLTKDEYERHNSYTCEATHKTSTSPIVKSFN | |
| | RNEC | |

CDR, VH, and VL sequences for each of antibodies A, B, C, D, E, F, G, H, J, and K are shown in Table 2.

TABLE 6
Table 2A
Antibody A Sequences

| | | SEQ ID |
|---|---|---|
| Description | Sequence | NO: |
| HCDR1 amino acid sequence (IMGT | GYTFTDYE | 143 |
| definition) | | |
| HCDR2 amino acid sequence (IMGT | VDPETGGT | 144 |
| definition) | | |
| HCDR3 amino acid sequence (IMGT | ARHGGSFYAMDY | 145 |
| definition) | | |
| LCDR1 amino acid sequence (IMGT | QDVSTA | 146 |
| definition) | | |
| LCDR2 amino acid sequence (IMGT | WAS | 147 |
| definition) | | |
| LCDR3 amino acid sequence (IMGT | QQHYSTPPT | 148 |
| definition) | | |
| VH amino acid sequence (predicted | QVQLQQPGAELVRPGASVTLSC | 149 |
| mature) | KASGYTFTDYEMHWVKQTPVHG | |
| | LEWIGGVDPETGGTGYNQKFRG | |
| | KAILTADKSSSTAYMELRSLTS | |
| | EDSAVYYCARHGGSFYAMDYWG | |
| | QGTSVTVSS | |
| VL amino acid sequence (predicted | QVQLQQPGAELVRPGASVTLSC | 150 |
| mature) | KASGYTFTDYEMHWVKQTPVHG | |
| | LEWIGGVDPETGGTGYNQKFRG | |
| | KAILTADKSSSTAYMELRSLTS | |
| | EDSAVYYCARHGGSFYAMDYWG | |
| | QGTSVTVSS | |

TABLE 7
Table 2B
Antibody B Sequences

| | | SEQ ID |
|---|---|---|
| Description | Sequence | NO: |
| HCDR1 amino acid sequence (IMGT | GFTFSSYG | 151 |
| definition) | | |
| HCDR2 amino acid sequence (IMGT | ISRGSIYT | 152 |
| definition) | | |
| HCDR3 amino acid sequence (IMGT | ARLSLYDYDGARYTMDY | 153 |
| definition) | | |
| LCDR1 amine acid sequence (IMGT | QDVGTA | 154 |
| definition) | | |
| LCDR2 amino acid sequence (IMGT | WAS | 155 |
| definition) | | |
| ICDR3 amino acid sequence (IMGT | QQYSSYHT | 156 |
| definition) | | |
| VH amino acid sequence (predicted | EVQLVESGGDLVKPGGSLKLSC | 157 |
| mature) | AASGFTFSSYGMSWVRQTPDKR | |
| | LEWVATISRGSIYTYYPDSVKG | |
| | RFITSRDNAKNTLYLQMSSLKS | |
| | EDTAMYYCARLSLYDYDGARYT | |
| | MDYWGQGTSVIVSS | |
| VL amino acid sequence (predicted | DIVMTQSPKFMSTSVGDRVSIT | 158 |
| mature) | CKASQDVGTAVAWYQQKPGQSP | |
| | KILIYWASTRHTGVPDRFTGSG | |
| | SGTDFTLTISNVQSEDLADYFC | |
| | QQYSSYHTFGAGTKLELK | |

TABLE 8
Table 2C
Antibody C Sequences

| | | SEQ ID |
|---|---|---|
| Description | Sequence | NO: |
| HCDR1 amino acid sequence (IMGT | GYNFKSYG | 159 |
| definition) | | |
| HCDR2 amino acid sequence (IMGT | IYPGSGNT | 160 |
| definition) | | |
| HCDR3 amino acid sequence (IMGT | ARTYGYDSFPWFAY | 161 |
| definition) | | |
| LCDR1 amino acid sequence (IMGT | KSLLHSNGNTY | 162 |
| definition) | | |
| LCDR2 amino acid sequence (IMGT | RVS | 163 |
| definition) | | |
| LCDR3 amino acid sequence (IMGT | MQHLEYPFT | 164 |
| definition) | | |
| VH amino acid sequence (predicted | QVQLKQSGAELARPGASVKLSC | 165 |
| mature) | KASGYNFKSYGISWVKQRTGQG | |
| | LEWIGEIYPGSGNTYYNEKLKG | |
| | KATLTADKSSSTAYMELRSLTS | |
| | EDSAVYFCARTYGYDSFPWFAY | |
| | WGQGTLVTVSS | |
| VL amino acid sequence (predicted | DIVMTQAAPSVPVTPGESVSIS | 166 |
| mature) | CRSSKSLLHSNGNTYLYWFLQR | |
| | PGQSFQLLIYRVSNLASGVPDR | |
| | FSGSGSGTAFTLRISRVEAEDE | |
| | GVYYCMQHLEYPFTFGAGTKLE | |
| | IK | |

TABLE 9
Table 2D
Antibody D Sequences

| | | SEQ ID |
|---|---|---|
| Description | Sequence | NO: |
| HCDR1 amino acid sequence (IMGT | GYTFTDYE | 167 |
| definition) | | |
| HCDR2 amino acid sequence (IMGT | IDPETGGT | 168 |
| definition) | | |
| HCDR3 amino acid sequence (IMGT | TRHGSYAMDY | 169 |
| definition) | | |
| LCDR1 amino acid sequence (IMGT | QDVSTA | 170 |
| definition) | | |
| LCDR2 amino acid sequence (IMGT | WAS | 171 |
| definition) | | |
| LCDR3 amino acid sequence (IMGT | QQHYSTPPT | 172 |
| definition) | | |
| VH amino acid sequence (predicted | EVQLQQSGAELVRPGASVTLSC | 173 |
| mature) | KASGYTFTDYEMHWVQQTPVHG | |
| | LEWIGGIDPETGGTGYNQKFKG | |
| | KATLTADKSSSTAYMELRSLTS | |
| | EDSAVYFCTRHGSYAMDYWGQG | |
| | TSVTVSS | |
| VL amino acid sequence (predicted | DIVMTQSPKFMSTSVGDRVSIT | 174 |
| mature) | CKASQDVSTAVAWYQQKPGQSP | |
| | KLLIYWASTRHTGVPDRFTGSG | |
| | SGTDYTLTISSVQAEDLALYYC | |
| | QQHYSTPPTFGGGTRLEIK | |

TABLE 10
Table 2E
Antibody E Sequences

| | | SEQ ID |
|---|---|---|
| Description | Sequence | NO: |
| HCDR1 amino acid sequence (IMGT | GYTFTDYE | 175 |
| definition) | | |
| HCDR2 amino acid sequence (IMGT | IDPESGGT | 176 |
| definition) | | |
| HCDR3 amino acid sequence (IMGT | TRSGYYGSPLLDY | 177 |
| definition) | | |
| LCDR1 amino acid sequence (IMGT | SSVSSSY | 178 |
| definition) | | |
| LCDR2 amino acid sequence (IMGT | STS | 179 |
| definition) | | |
| LCDR3 amino acid sequence (IMGT | HQYHRSPPT | 180 |
| definition) | | |
| VH amino acid sequence (predicted | EVKLLESGAELVRPGASVTLSC | 181 |
| mature) | KASGYTFTDYEMHWVKQTPVHG | |
| | LEWIGGIDPESGGTGYNQKFKG | |
| | KAILTADKSSSTAYMELRSLTS | |
| | EDSAVYFCTRSGYYGSPLLDYW | |
| | GQGTTLTVSS | |
| VL amino acid sequence (predicted | QIVLSQSPAIMSASLGERVTMT | 182 |
| mature) | CTASSSVSSSYLHWYQQKPGSS | |
| | PKLWIYSTSNLASGVPARFSGS | |
| | GSGTSYSLTISSMEAEDAATYY | |
| | CHQYHRSPPTFGAGTKLEIK | |

TABLE 11
Table 2F
Antibody F Sequences

| | | SEQ ID |
|---|---|---|
| Description | Sequence | NO: |
| HCDR1 amino acid sequence (IMGT | GYTFTDYY | 183 |
| definition) | | |
| HCDR2 amino acid sequence (IMGT | IFPGSGST | 184 |
| definition) | | |
| HCDR3 amino acid sequence (IMGT | ADYGSSYRYFDV | 185 |
| definition) | | |
| LCDR1 amino acid sequence (IMGT | SSVSY | 186 |
| definition) | | |
| LCDR2 amino acid sequence (IMGT | ATS | 187 |
| definition) | | |
| LCDR3 amino acid sequence (IMGT | QQWSSNPLT | 188 |
| definition) | | |
| VH amino acid sequence (predicted | QVQLKESGPELVKPGASVKISC | 189 |
| mature) | KASGYTFTDYYINWVKQRPGQG | |
| | LEWIGWIFPGSGSTYYNEKFKG | |
| | KATLTVDKSSSTAYMLLSSLTS | |
| | EDSAVYFCADYGSSYRYFDVWG | |
| | ACTTVTVSS | |
| VL amino acid sequence (predicted | DIVMTQSPAILSASPGEKVTMT | 190 |
| mature) | CRASSSVSYMHWYQQKPGSSPK | |
| | PWIYATSYLASGVPARFSGSGS | |
| | GTSYSLTIGRVEAEDAATYYCQ | |
| | QWSSNPLTFGAGTKLELK | |

TABLE 12
Table 2G
Antibody G Sequences

| | | SEQ ID |
|---|---|---|
| Description | Sequence | NO: |
| HCDR1 amino acid sequence (IMGT | GYTFTDYE | 191 |
| definition) | | |
| HCDR2 amino acid sequence (IMGT | IDPETGGT | 192 |
| definition) | | |
| HCDR3 amino acid sequence (IMGT | TRWGAITSEVALRGFAY | 193 |
| definition) | | |
| LCDR1 amino acid sequence (IMGT | QSLLNSGNQKNY | 194 |
| definition) | | |
| LCDR2 amino acid sequence (IMGT | GAS | 195 |
| definition) | | |
| LCDR3 amino acid sequence (IMGT | QNDHSYPPT | 196 |
| definition) | | |
| VH amino acid sequence (predicted | QVQLKQSGAELVRPGASVTLSC | 197 |
| mature) | KASGYTFTDYEMHWVKQTPVHG | |
| | LENIGGIDPETGGTAYNQKFKG | |
| | KAILTADKSSSTAYMELRSLTS | |
| | EDSAVYYCTRWGAITSFVALRG | |
| | FAYWGQGTLVTVSS | |
| VL amino acid sequence (predicted | DIQMTQSPSSLSVSAGEKVTMS | 198 |
| mature) | CKSSQSLLNSGNQKNYLAWYQQ | |
| | KPGQPPKLLIYGASTRESGVPD | |
| | RFTGSGSGTDFTLTISSVQAED | |
| | LAVYYCQNDHSYPPTFGAGTKL | |
| | ELK | |

TABLE 13
Table 2H
Antibody H Sequences

| | | SEQ ID |
|---|---|---|
| Description | Sequence | NO: |
| HCDR1 amino acid sequence (IMGT | GFTFSSYG | 199 |
| definition) | | |
| HCDR2 amino acid sequence (IMGT | ISRRSIYT | 200 |
| definition) | | |
| HCDR3 amino acid sequence (IMGT | ARLSLYDYDGARYTMDY | 201 |
| definition) | | |
| LCDR1 amino acid sequence (IMGT | QDVGTA | 202 |
| definition) | | |
| LCDR2 amino acid sequence (IMGT | WAS | 203 |
| definition) | | |
| LCDR3 amino acid sequence (IMGT | QQYSSYHT | 204 |
| definition) | | |
| VH amino acid sequence (predicted | EVKLVESGGDLVKPGGSLKLSC | 205 |
| mature) | AASGFTFSSYGMSWVRQTPDKR | |
| | LEWVATTSRRSIYTYYPDSVQG | |
| | RFTISRDNAKNTLYLQMSSLKS | |
| | EDTAMYYCARLSLYDYDGARYT | |
| | MDYWGQGTSVTVSS | |
| VL amino acid sequence (predicted | DIKMTQSPKFMSTSVGDRVSIT | 206 |
| mature) | CKASQDVGTAVAWYQQKPGQSP | |
| | KLLIYWASTRHTGVPDRFTGSG | |
| | SGTDFTLTISNVQSEDLADYFC | |
| | QQYSSYHTFGAGTKLEIK | |

TABLE 14
Table 2I
Antibody J Sequences

| | | SEQ ID |
|---|---|---|
| Description | Sequence | NO: |
| HCDR1 amino acid sequence (IMGT | GYTFTSYW | 207 |
| definition) | | |
| HCDR2 amino acid sequence (INGT | INPSNGGT | 208 |
| definition) | | |
| HCDR3 amino acid sequence (IMGT | ARGGIYYGNYDYYAMDY | 209 |
| definition) | | |
| LCDR1 amino acid sequence (IMGT | KSLLHSNGNTY | 210 |
| definition) | | |
| LCDR2 amino acid sequence (IMGT | RVS | 211 |
| definition) | | |
| LCDR3 amino acid sequence (IMGT | MQHLEYPFT | 212 |
| definition) | | |
| VH amino acid sequence predicted | QVHLQQSGTELVKPGASVKLSC | 213 |
| mature) | EASGYTFTSTWMHWVKQRPGQG | |
| | LEWIGNINPSNGGTNYNEKFKS | |
| | KATLTVDKSSSTAYMQLSSLTS | |
| | EDSAVYYCARGGIYYGNYDYYA | |
| | MDYWGQGTSVTVSS | |
| VL amino acid sequence (predicted | DIVMTQAAPSVPVTPGESVSIS | 214 |
| mature) | CRSSKSLLHSNGNTYLYWFLQR | |
| | PGQSPQLLIYRVSNLASGVPDR | |
| | FSGSGSGTAFTLRISRVEAEDV | |
| | GVYYCMQHLEYPFTFGGGTKLE | |
| | IK | |

TABLE 15
Table 2J
Antibody K Sequences

| | | SEQ ID |
|---|---|---|
| Description | Sequence | NO: |
| HCDR1 amino acid sequence (IMGT | GYAFTNYL | 215 |
| definition) | | |
| HCDR2 amino acid sequence (IMGT | INPGSGGT | 216 |
| definition) | | |
| HCDR3 amino acid sequence (IMGT | ARWGFYYAMDY | 217 |
| definition) | | |
| LCDR1 amino acid sequence (IMGT | QEISGY | 218 |
| definition) | | |
| LCDR2 amino acid sequence (IMGT | AAS | 219 |
| definition) | | |
| LCDR3 amino acid sequence (IMGT | LQYASYPLT | 220 |
| definition) | | |
| VH amino acid sequence (predicted | QVQLKQSGAELVRPGTSVKVSC | 221 |
| mature) | KASGYAFTNYLIEWVKQRPGQG | |
| | LEWIGVINPGSGGTNYNEKFKG | |
| | KATLTADKSSSTAYMQLSSLTS | |
| | EDSAVYFCARWGFYYAMDYWGQ | |
| | GTSVTVSS | |
| VL amino acid sequence (predicted | DIVMTQSPSSLSASLGERVSLT | 222 |
| mature) | CRASQEISGYLSWLQQKPDGTI | |
| | KRLIYAASTLDSGVPKRFSGSR | |
| | SGSDYSLTISSLESEDFADYYC | |
| | LQYASYPLTFGAGTKLEIK | |

### Example 10

Generation of Anti-AQP3 Antibodies in Rabbit

In this Example 10, anti-AQP3 antibodies were generated by immunizing a rabbit with eight oligopeptides that are located on the extracellular portion of AQP3. Table 3 shows the sequence of the oligopeptides, their respective SEQ ID NO, and their location in AQP3.

TABLE 16
Table 3

| OLIGOPEPTIDE SEQUENCE | SEQ ID NO's |
|---|---|
| Loop A | |
| SRGTHGGFL | SEQ ID NO: 2 |
| Loop C | |
| DAIWHFADNQLFVS | SEQ ID NO: 223 |
| LFVSGPNGTA | SEQ ID NO: 224 |
| SGPNGTAGIFATYPS | SEQ ID NO: 94 |
| ATYPSGHLDM | SEQ ID NO: 1 |
| Loop E | |
| RDFGPRLFTALAGWG | SEQ ID NO: 226 |
| RLFTALAGWGS | SEQ ID NO: 227 |
| TALAGWGSAVFTTG | SEQ ID NO: 228 |

These eight oligopeptide were generated as synthetic oligopeptide according to standard methods. A mixture of the eight peptides, together with cells overexpressing AQP3, was used to immunize a rabbit.

The rabbit inoculated with the mixture of peptides and AQP3 overexpressing cells according to standard procedures. After approximately two weeks the rabbit was boosted with the same immunogens and after two more similar boosts, the rabbit was sacrificed and the spleen and bone marrow were collected for mRNA isolation. An antibody gene phage library was constructed using this mRNA and enriched for AQP3 phage binders specifically by binding to the peptides and cells that overexpress AQP3. Antibody fragments (Fabs) were produced from the enriched library and subjected to ELISA peptide binding studies and flow cytometry analysis (FACS). The ELISA studies were conducted according to known procedures and used each of the eight peptides individually as reagents to test the Fab binding. The FACS analysis was conducted according to standard procedures using AQP3 expressing CHO cells.

This antibody production plan generated twenty-eight clones that produce Fabs that bind to SEQ ID NO:1 and cell-expressed AQP3. Four clones were selected to conduct further binding experiments (Example 11-13) and the activity experiments (Examples 14 and 15). These four clones bound specifically to SEQID NO:1 in ELISA screens and AQP3 expressing CHO cells in FACS screens.

### Example 11

Anti AQP3 antibodies bound to an oligopeptide from the extracellular portion of AQP3

Four clones from Example 10, SC—F8, BC—H9, BC—B10, and SC—B6, were subjected to binding studies. These clones were first converted into immunoglobulin G (IgG) and their binding to SEQ ID NO:1 was confirmed using ELISA.

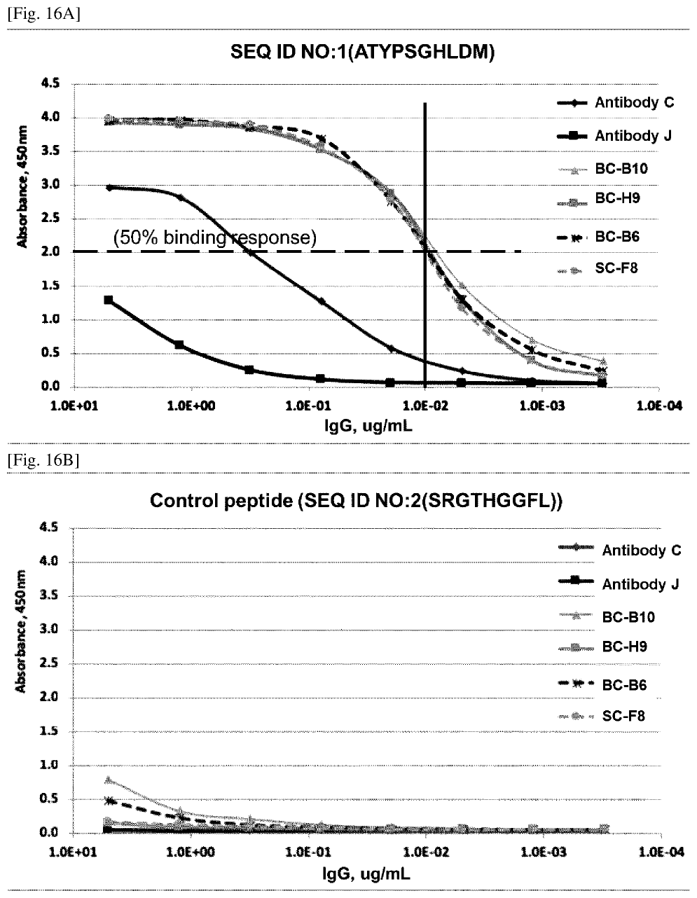

FIG. 16 are graphs showing the result of an ELISA analysis testing the binding property of SC—F8 (circles), BC—H9 (gray squares), BC—B10 (triangles), and SC—B6 (exes)) compared to anti AQP3 antibodies (antibody C (diamonds) and antibody J (black squares)) to a peptide having the amino acid sequence of SEQ ID NO:1 (FIG. 16A) or a control peptide SEQ ID NO:2, a Loop A peptide (FIG. 16B). Although both oligopeptides are part of the extracellular portion of AQP3, oligopeptide 5 is located in Loop C and oligopeptide 1 is located in Loop A.

Also shown is a dashed line indicating the 50% binding response for SC—F8, BC—H9, BC—B10, and SC—B6. The 50% binding response is roughly equivalent to the affinity an antibody has to its epitope. More specifically and according to certain embodiments, affinity is defined by a 50% maximal binding response in a biochemical plate-based binding assay with the peptide.

More specifically, as shown in FIG. 16A, the amount of antibody needed for the 50% binding response is 0.01 μg/mL for SC—F8, BC—H9, BC—B10, and SC—B6 compared to approximately 0.1 μg/mL 50% binding response for antibody C and greater than 1.0 μg/mL for antibody J. Thus, the SC—F8, BC—H9, BC—B10, and SC—B6 bind to SEQ ID NO:1 at 50% response at concentration of roughly 0.01 μg/mL, which for an IgG translates into about 0.06 nM (60 pM). According to certain embodiments, each of the four clones bind with an affinity tighter than 100 pM.

Thus, the four clones bound specifically to a peptide from Loop C of the extracellular portion of AQP3 and not to a peptide from Loop A.

### Example 12

Anti AQP3 Antibodies Bound to AQP3-Expressing Cells

In this Example, mouse keratinocytes (PAM212) and human keratinocytes (HaCaT) as AQP3-expressing cells, were used to test the binding properties of four clones, SC—F8, BC—H9, BC—B10, and SC—B6.

PAM212 cells were reacted with each anti AQP3 antibody at concentrations of none, 1 ng/mL, 10 ng/mL, 100 ng/mL, 1 μg/mL, or 10 μg/mL at 4° C. for 1 hour. After washing the cells, a fluorescent-labeled secondary antibody was added and the reaction was allowed to occur additionally for 1 hour (at 4° C.). By measuring the fluorescence intensity, the binding property of each anti AQP3 antibody to cells was obtained.

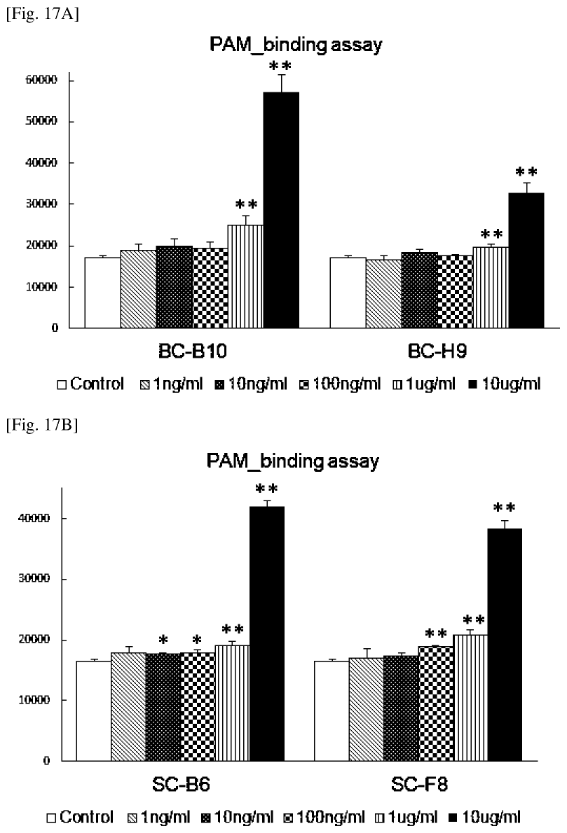

The result of the above experiment is shown in FIG. 17A for clones BC—H9 and BC—B10 and FIG. 17B for SC—F8 and SC—B6. In FIG. 17A, B the vertical axis represents fluorescence intensity, and the mean fluorescence intensity of each sample is represented by bar height together with standard error.

Each anti AQP3 antibody clone bound to PAM212 cells with increasing intensity as the concentration increased to the point where the BC—B10, SC—F8, and SC—B6 10 μg/mL concentration bound with at least double the intensity over no antibody (in the drawing, * represents the presence of a significant difference of P<0.05 and ** represents the presence of a significant difference of P<0.01, when compared to the no antibody control).

Next in this Example, HaCaT cells were used to test the binding properties of the four clones, SC—F8, BC—H9, BC—B10, and SC—B6.

HaCaT cells were reacted with each anti AQP3 antibody at concentrations of none, 1 ng/mL, 10 ng/mL, 100 ng/mL, 1 μg/mL, or 10 μg/mL at 4° C. for 1 hour. After washing the cells, a fluorescent-labeled secondary antibody was added and the reaction was allowed to occur additionally for 1 hour (at 4° C.). By measuring the fluorescence intensity, the binding property of each anti AQP3 antibody to cells was obtained.

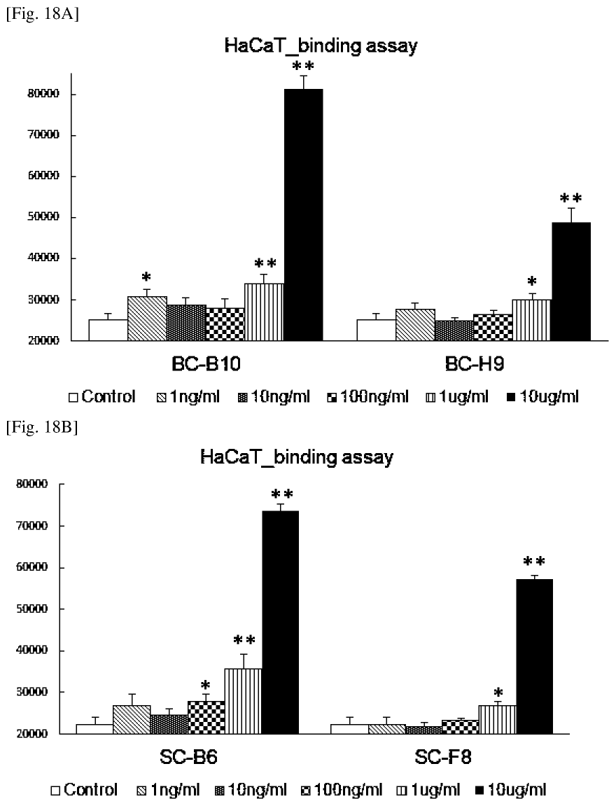

The result of the above experiment is shown in FIG. 18A for clones BC—H9 and BC—B10 and FIG. 18B for SC—F8 and SC—B6. In FIG. 18A, B, the vertical axis represents fluorescence intensity, and the mean fluorescence intensity of each sample is represented by bar height together with standard error.

Each anti AQP3 antibody clone bound to HaCaT cells with increasing intensity as the concentration increased to the point where the BC—B10, SC—F8, and SC—B6 10 μg/mL concentration bound with well more than twice the intensity over no antibody (in the drawing, * represents the presence of a significant difference of P<0.05 and ** represents the presence of a significant difference of P<0.01, when compared to the no antibody control).

From all cases in which any of antibodies from the clones were used, a clear increase in fluorescence intensity was recognized compared to the control. Thus, from the experiments described above, several anti AQP3 antibodies of the present invention were found to bind to mouse keratinocytes cells (PAM212 cells) and human keratinocytes cells (HaCaT cells).

### Example 13

Anti AQP3 Antibodies Bound Specifically to AQP3-Expressing Cells

In this Example, the specificity of the anti-AQP3 antibodies binding to mouse keratinocytes (PAM212) was tested by blocking expression of AQP3 with a small interfering RNA (siRNA) specific to the AQP3 mRNA. PAM212 cells were transfected with either an siRNA AQP3 or a control siRNA. The siRNA preparation and transfection were conducted according to those techniques known in the field. More specifically, for this Example 13, the PAM212 cell lines containing the AQP3 siRNA and the control siRNA were constructed by transfecting either mouse AQP3 or non-targeting-siRNA using Lipofectamine 2000 (Invitrogen) with (ON-TARGET plus SMART pool, Thermo Scientific). The mouse AQP3 siRNA SMART pool contained four RNAs: UCGUUGACCCUUAUAACAA (SEQ ID NO:111); GGGCUUCAAUU-CUGGCUAU (SEQ ID NO:112); CAUUAGGCGAUGUGAGGUU (SEQ ID NO:113); GCUGAAGUCCAGGUCGUAA (SEQ ID NO:114). The non-targeting siRNA SMART pool contained four RNAs: UGGUUUACAUGUCGACUAA (SEQ ID NO:115); UGGUUUACAUGUUGUGUGA (SEQ ID NO:116); UGGUUUACAU-GUUUUCUGA (SEQ ID NO:117); UGGUUUACAUGUUUUCCUA (SEQ ID NO:118).

The resulting siRNA AQP3 cell line had 10% of the AQP3 expression compared to the control siRNA cell line. The siRNA AQP3 and control siRNA PAM212 cells were reacted with SC—F8, BC—H9, BC—B10, and SC—B6 at the chosen concentration 1 μg/mL at 4° C. for 1 hour. After washing the cells, a fluorescent-labeled secondary antibody was added and the reaction was allowed to occur additionally for 1 hour (at 4° C.). By measuring the fluorescence intensity, the binding property of each anti AQP3 antibody to the two cell lines was obtained.

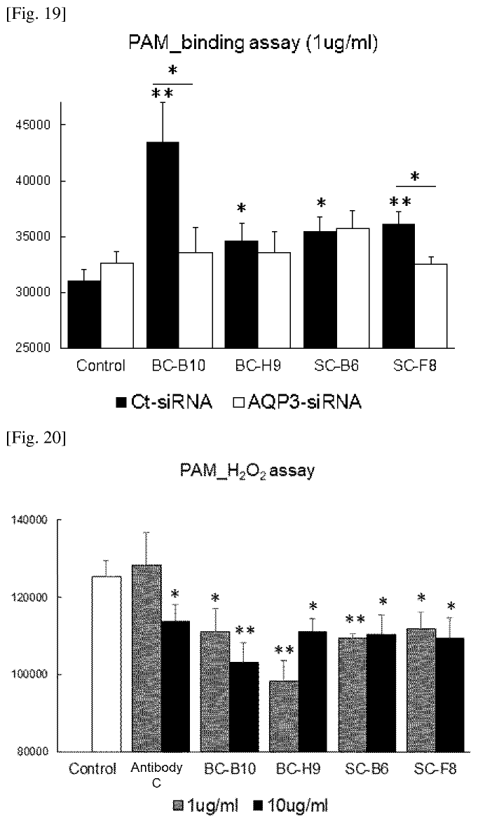

The result of the above experiment is shown in FIG. 19, wherein the vertical axis represents fluorescence intensity, and the mean fluorescence intensity of each sample is represented by bar height together with standard error.

Each anti AQP3 antibody clone at the concentration of 1 μg/mL bound to PAM212 cells with similar intensity as seen in Example 12. In particular, the binding of each of the antibody clones had a statistically significant higher binding over the no antibody control, in the drawing, * represents the presence of a significant difference of P<0.05 and ** represents the presence of a significant difference of P<0.01, when compared to the no antibody control. Further, in the down regulated AQP3 PAM212 cells, FIG. 19 indicates approximately a two fold decrease in fluorescence intensity in one clone, BC—B10, and a statistically significant decrease in the binding of SC—F8 clone (* represents the presence of a significant difference of P<0.05 when comparing the binding of siRNA AQP3 PAM212 cells to siRNA control PAM212 cells.)

Thus, there is a significant decrease in the binding of the anti-AQP3 antibody clones in a murine keratinocyte cell line that has 10% expression of AQP3 when compared to a cell line that has full expression of AQP3.

### Example 14

Anti AQP3 Antibodies Inhibited Hydrogen Peroxide Permeation

The ability of the four AQP3 antibody clones SC—F8, BC—H9, BC—B10, SC—B6 and antibody C to inhibit cell permeation of hydrogen peroxide (H₂O₂) was tested in both murine (PAM212) and human keratocytes (HaCaT).

### Murine Keratocytes

PAM212 cells, seeded in a 96-well plate were reacted with each anti AQP3 antibody at concentrations of 1 μg/mL, 10 μg/mL, or a non-specific antibody at 10 μg/mL, and co-culture was additionally continued overnight. To the culture, H₂O₂ (100 μM) was added and after incubating the cells for one hour at 37° C., the amount of reactive oxygen species (ROS) in the cells was measured. The ROS amount in the cells was evaluated by, after staining the cells by adding CM-H2DCFDA reagent (Invitrogen, 50 μM, for 20 minutes), measuring the fluorescence intensity derived from CM2DCF before and after the addition. If hydrogen peroxide, as one kind of ROS, permeates into the cell, it is possible to perform a measurement in which increased fluorescence intensity is taken as an indicator of an increased ROS amount in cells.

The result of the above experiment is shown in FIG. 20, wherein the vertical axis represents fluorescence intensity, and the mean fluorescence intensity of each sample is represented by bar height together with standard error. At both 1 μg/mL and 10 μg/mL concentrations for each of the four clones there was statistically significant decrease in the fluorescence intensity from ROS (in the drawing, * represents the presence of a significant difference of P<0.05 and ** represents the presence of a significant difference of P<0.01, when compared to the no antibody control). Thus, this experiment shows that the four AQP3 antibodies clones SC—F8, BC—H9, BC—B10, and SC—B6 inhibited permeation of H₂O₂ into PAM212 cells compared to a control antibody.

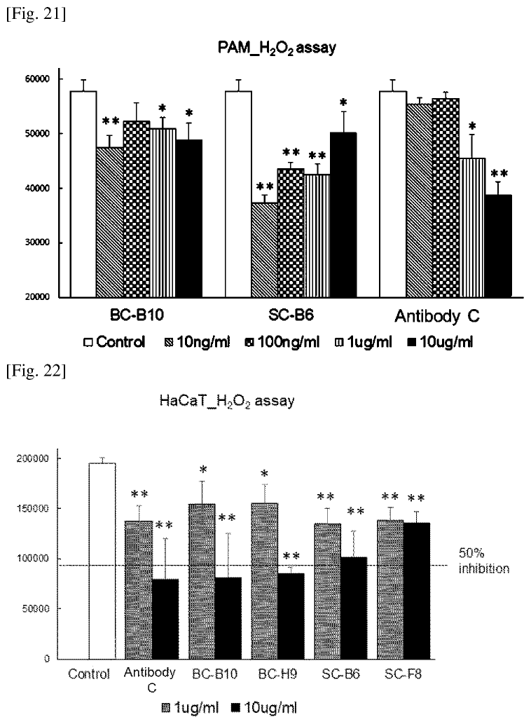

FIG. 21 shows the results from the H₂O₂ uptake inhibition studies using step order of magnitude increasing concentrations of two of the AQP3 antibodies clones (BC—B10 and SC—B6) in PAM212 cells. The concentrations used for this figure were 10 ng/mL, 100 ng/mL, 1 μg/mL, or 10 μg/mL. The specifics of the experiments were the same as above and as described for the experiment results shown in FIG. 20. The SC—B6 anti-AQP3 antibody clone had an almost 2-fold decrease in H₂O₂ uptake at the 10 ng/mL concentration when compared to the no antibody control. Further, all of the antibody concentrations for the SC—B6 anti-AQP3 antibody clone and all but one antibody concentrations for BC—B10 had a statistically significant decrease in the fluorescence intensity from ROS compared to the no antibody control (in the drawing, * represents the presence of a significant difference of P<0.05 and ** represents the presence of a significant difference of P<0.01, when compared to the no antibody control). Thus, this experiment shows that BC—B10 and SC—B6 substantially inhibited permeation of H ₂O₂ into PAM212 cells.

### Human Keratocytes

HaCaT cells, seeded in a 96-well plate were reacted with each anti AQP3 antibody at concentrations of 1 μg/mL, 10 μg/mL, or a non-specific antibody at 10 μg/mL, and co-culture was additionally continued overnight. To the culture, H₂O₂ (100 μM) was added and after incubating the cells for one hour at 37° C., the amount of reactive oxygen species (ROS) in the cells was measured. The ROS amount in the cells was evaluated by, after staining the cells by adding CM-H2DCFDA reagent (Invitrogen, 50 μM, for 20 minutes), measuring the fluorescence intensity derived from CM2DCF before and after the addition. If hydrogen peroxide, as one kind of ROS, permeates into the cell, it is possible to perform a measurement in which increased fluorescence intensity is taken as an indicator of an increased ROS amount in cells.

The result of the above experiment is shown in FIG. 22, wherein the vertical axis represents fluorescence intensity, and the mean fluorescence intensity of each sample is represented by bar height together with standard error. At both 1 μg/mL and 10 μg/mL concentrations for each of the four clones there was statistically significant decrease in the fluorescence intensity from ROS (in the drawing, * represents the presence of a significant difference of P<0.05 and ** represents the presence of a significant difference of P<0.01, when compared to the no antibody control). Thus, this experiment shows that the four AQP3 antibodies clones SC—F8, BC—H9, BC—B10, and SC—B6 inhibited permeation of H₂O₂ into HaCaT cells. Also shown in this figure is the 50% dashed line indicating a 50% reduction in H₂O₂ uptake in cells treated with the anti AQP3 antibodies compared to the no antibody control. Two of the clones (BC—B10 and BC—H9) block more than 50% of H₂O₂ uptake in the human keratocytes at the 10 ng/mL concentration when compared to the no antibody control. Further, the SC—B6 anti-AQP3 antibody clone had almost 50% reduction in H₂O₂ uptake at the 10 ng/mL concentration when compared to the no antibody control.

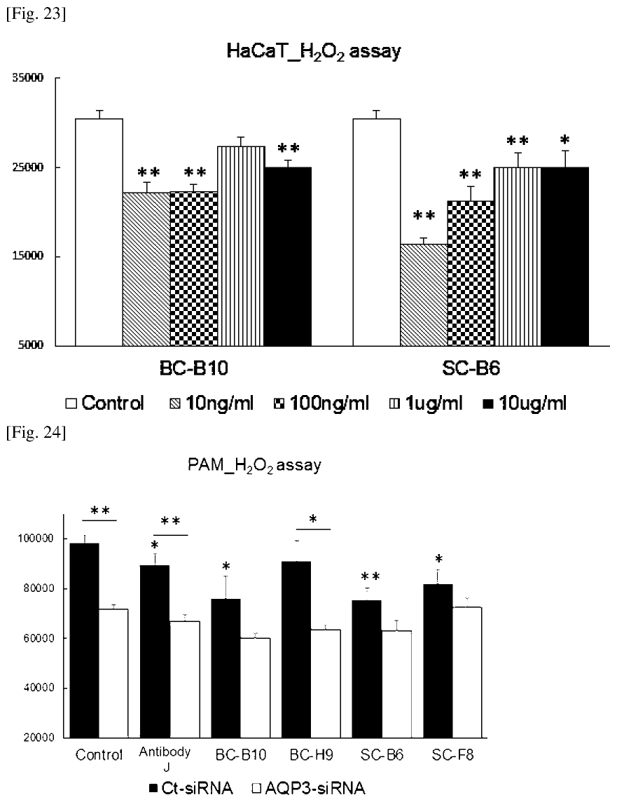

FIG. 23 shows the results from the H₂O₂ uptake inhibition studies using increasing order of magnitude concentrations of BC—B10 and SC—B6 in HaCaT cells. The concentrations used for this figure were 10 ng/mL, 100 ng/mL, 1 μg/mL, or 10 μg/mL. The specifics of the experiments were the same as above and as described for the experiment results shown in FIG. 22. As shown in FIG. 23, all of the antibody concentrations for the SC—B6 anti-AQP3 antibody clone and all but one antibody concentrations for BC—B10 had a statistically significant decrease in the fluorescence intensity from ROS compared to the no antibody control (in the drawing, * represents the presence of a significant difference of P<0.05 and ** represents the presence of a significant difference of P<0.01, when compared to the no antibody control).

Thus, this Example shows that BC—B10, BCH9, and SC—B6 substantially inhibited permeation of H₂O₂ into HaCaT cells.

### Example 15

The inhibition of hydrogen peroxide permeation seen in Example 14 was specific to the presence of AQP3

In this Example, the ability of the anti-AQP3 antibodies SC—F8, BC—H9, BC—B10, and SC—B6 to inhibit H₂O₂ uptake in mouse keratinocytes (PAM212) was tested by reducing expression of AQP3 with a small interfering RNA (siRNA) specific to the AQP3 mRNA.

PAM212 cells were transfected with either an siRNA AQP3 or a control siRNA. The siRNA preparation and transfection were conducted according to those techniques known in the field. More specifically, for this Example 15, the PAM212 cell lines containing the AQP3 siRNA and the control siRNA were constructed by transfecting either mouse AQP3 or non-targeting-siRNA using Lipofectamine 2000 (Invitrogen) with (ON-TARGET plus SMART pool, Thermo Scientific). The mouse AQP3 siRNA SMART pool contained four RNAs: UCGUUGACCCUUAUAACAA (SEQ ID NO:111); GGGCUUCAAUUCUGGCUAU (SEQ ID NO:112); CAUUAGGCGAU-GUGAGGUU (SEQ ID NO:113); GCUGAAGUCCAGGUCGUAA (SEQ ID NO:114). The non-targeting siRNA SMART pool contained four RNAs: UGGUUUA-CAUGUCGACUAA (SEQ ID NO:115); UGGUUUACAUGUUGUGUGA (SEQ ID NO:116); UGGUUUACAUGUUUUCUGA (SEQ ID NO:117); UGGUUUACAU-GUUUUCCUA (SEQ ID NO:118).

The resulting siRNA AQP3 cell line was shown to have 10% of the AQP3 expression compared to the control siRNA cell line. The siRNA AQP3 and control siRNA PAM212 cells were reacted with each anti AQP3 antibody at the chosen concentration 1 μg/mL at 4° C. for 1 hour. The H₂O₂ uptake permeability was carried out as described in Example 14.

The result of the above experiment is shown in FIG. 24, wherein the vertical axis represents fluorescence intensity, and the mean fluorescence intensity of each sample is represented by bar height together with standard error. There are a few interesting points to note about the results. First, there was a statistically significant decrease in H₂O₂ permeability when the siRNA AQP3 was used, in comparison to the siRNA control group (** represents the presence of a significant difference of P<0.01 when comparing the permeability between the two groups.) This indicates that the H₂O₂ permeability in this assay is AQP3-dependent. Second, and as a repeat of Example 14, SC—F8, BC—B10, and SC—B6 decreased the uptake of H₂O₂ in a statistically significant manner (in the drawing, * represents the presence of a significant difference of P<0.05 and ** represents the presence of a significant difference of P<0.01, when compared to the no antibody control.) Third, the anti-AQP3 antibodies did not significantly affect H₂O₂ permeation in the cells with reduced AQP3 expression, indicating that the effect of the antibodies is due to their interaction with AQP3.

Thus, there was no significant decrease in H₂O₂ permeability when using the anti-AQP3 antibody clones beyond the already lower uptake due to the presence of an AQP3 siRNA.

### Example 16

Binding analysis of the specific amino acid sequences of SEQ ID NO:1 important for binding to anti AQP3 antibody clones

To determine the important amino acid residues of an epitope in Loop C involved in binding to specific AQP3 antibody clones, peptides of varying lengths were produced. The sequences of these peptides are shown in column 2 of Table 4.

The various peptides and AQP3 antibody clones were subjected to ELISA binding analyses. Each peptide was diluted to 1 mg/mL in water except for SEQ ID NO:97 that showed precipitation, thus it was initially diluted in DMSO, then all were further diluted to 1 μg/mL in PBS. The microtiter wells (Costar 2690) were coated with 50 μL of each peptide at 4° C. overnight. The wells were washed 3 times with PBS and blocked with 100 μL of 1% BSA/PBS at 37° C. for 1 hour. Each antibody was adjusted to 1 μg/mL in 1% SBA/PBS as above then serially diluted 1:5 in 1% BSA/PBS. The blocker was discarded and the wells were incubated with 50 μL of antibodies at 37° C. for 1.5 hours. The wells were washed 3 times with PBS and mouse antibodies were detected with 50 μL of goat anti-mouse IgG (H+L) HRP conjugate (ThermoFisher 31438) (1:5,000 in 1% BSA/PBS) and rabbit antibodies were detected with 50 μL of goat anti-rabbit IgG (H+L) HRP conjugate (ThermoFisher 31462) (1:5,000 in 1% BSA/PBS) at 37° C. for 1 hour. The wells were washed 3 times with PBS and developed with 50 μL of HRP substrate at RT for 5 min then stopped with 50 μL of 2N sulfuric acid and binding was measured with a plate reader.

Table 4 shows the name of each peptide (column 1), the peptide sequence (column 2), and the amount of binding signal from each antibody clone has as measured by plate reader. The higher values correspond to more antibody binding. (clones from left to right: antibody C, antibody J, SC—F8, BC—H9, BC—B10, SC—B6, and SC—B10).

As shown in Table 4, different AQP3 antibody clones had different binding patterns to the peptides. In particular and as shown in the summary table, Table 5, the YPSGH (SEQ ID NO:90) residues were important for three of the five antibody clones (SC—F8, BC—H9, and SC—B6), the PS residues were important for one of the antibody clones (BC—B10) and the GHLDM (SEQ ID NO:91) residues were important for another of the antibody clones (SC—B10). For the antibody C antibody, FATYPSGHLD (SEQ ID NO:67) contained the major contact amino acids and the addition of TAGIF (SEQ ID NO:92) on the N terminus enhanced the binding to some extent. The antibody J antibody also shows the same trend albeit with very weak binding.

Interestingly, according to the binding data, the BC—B10 antibody clone only requires two amino acid residues to bind, PS, and these two residues are contained within Loop C. Also interesting was that the SC—B10 antibody binds to a complete unique amino acid sequence of Loop C, when compared to the other antibody clones.

In conclusion, three of the antibody clones had unique binding patterns to SEQ ID NO:1 in ELISA studies as shown in Tables 4 and 5. Table 5 is a summary of the binding data and in bold highlights the important residues for binding.

TABLE 4

| | OLIGOPEPTIDE | | | | | | | |
|---|---|---|---|---|---|---|---|---|
| SEQ ID NO | SEQUENCE | Antibody C | Antibody J | SC-F8 | BC-H9 | BC-B10 | SC-B6 | SC-B10 |
| SEQ ID NO: 94 | SGPNGTAGIFATYPS | 0.045 | 0.043 | 2.990 | 0.824 | 3.760 | 2.017 | 0.095 |
| SEQ ID NO: 1 | ATYPSGHLDM | 2.215 | 0.391 | 3.969 | 3.935 | 3.983 | 3.985 | 3.823 |
| SEQ ID NO: 96 | TYPSGHLDM | 1.792 | 0.260 | 3.915 | 3.986 | 3.955 | 4.000 | 3.962 |
| SEQ ID NO: 97 | YPSGHLDM | 1.579 | 0.086 | 3.945 | 3.938 | 3.964 | 3.965 | 3.842 |
| SEQ ID NO: 98 | PSGHLDM | 0.271 | 0.095 | 1.558 | 0.733 | 3.717 | 2.233 | 3.905 |
| SEQ ID NO: 99 | SGHLDM | 0.048 | 0.041 | 0.071 | 0.053 | 0.405 | 0.135 | 3.847 |
| SEQ ID NO: 100 | GHLDM | 0.043 | 0.043 | 0.060 | 0.048 | 0.123 | 0.097 | 3.640 |
| SEQ ID NO: 101 | ATYPSGHLD | 2.524 | 0.671 | 3.909 | 3.896 | 3.961 | 3.923 | 2.236 |
| SEQ ID NO: 102 | ATYPSGHL | 1.687 | 0.044 | 3.994 | 3.896 | 4.000 | 3.998 | 2.861 |
| SEQ ID NO: 103 | ATYPSGH | 0.047 | 0.045 | 3.810 | 3.164 | 3.911 | 3.440 | 0.226 |
| SEQ ID NO: 104 | ATYPSG | 0.044 | 0.044 | 1.771 | 0.627 | 3.540 | 0.915 | 0.085 |
| SEQ ID NO: 105 | TAGIFATYPSGHLDM | 2.806 | 0.723 | 3.998 | 3.922 | 3.986 | 3.959 | 3.830 |
| SEQ ID NO: 106 | AGIFATYPSGHLDM | 2.967 | 0.744 | 3.925 | 2.645 | 3.895 | 3.829 | 3.433 |
| SEQ ID NO: 107 | GIFATYPSGHLDM | 2.919 | 0.523 | 3.863 | 3.799 | 3.876 | 3.787 | 3.798 |
| SEQ ID NO: 108 | IFATYPSGHLDM | 2.796 | 0.594 | 3.912 | 3.760 | 3.884 | 3.691 | 3.652 |
| SEQ ID NO: 109 | FATYPSGHLDM | 2.798 | 0.513 | 3.915 | 3.748 | 3.922 | 3.873 | 3.420 |

TABLE 18

| ANTIBODY | | |
|---|---|---|
| CLONE | OLIGOPEPTIDE SEQUENCE | SEQ ID NO |
| Antibody C | SGPNGTAGIFATYPSGHLDM | SEQ ID NO: 110 |
| Antibody J | SGPNGTAGIFATYPSGHLDM | SEQ ID NO: 110 |
| SC-F8 | SGPNGTAGIFATYPSGHLDM | SEQ ID NO: 110 |
| BC-H9 | SGPNGTAGIFATYPSGHLDM | SEQ ID NO: 110 |
| BC-B10 | SGPNGTAGIFATYPSGHLDM | SEQ ID NO: 110 |
| SC-B6 | SGPNGTAGIFATYPSGHLDM | SEQ ID NO: 110 |
| SC-B10 | SGPNGTAGIFATYPSGHLDM | SEQ ID NO: 110 |

Table 5

### Example 17

Sequence Analysis of Anti AQP3 Antibody Clones

The amino acid sequence of heavy chain complementarity determining region1 (HCDR1), heavy chain complementarity determining region 2 (HCDR2), heavy chain complementarity determining region 3 (HCDR3), a light chain complementarity determining region 1 (LCDR1), a light chain complementarity determining region 2 (LCDR2), and a light chain complementarity determining region 3 (LCDR1) were determined for each of the 28 clones that were discovered using the protocol described in Example 10 and that are all SEQ ID NO:1 binders. The CDR consensus sequences are shown in Table 6. Individual CDR sequences for each of the clones is shown in Table 7. The heavy variable (VH) and light variable (VL) sequences for each of the clones is shown in Table 8.

TABLE 19
Table 6
CDR Consensus sequences (IMGT definition)-all 28 SEQ ID NO: 1
binders

| | | SEQ ID |
|---|---|---|
| Description | Sequence | NO: |
| HCDR1 amino acid sequence | X₁FSLX₂X₃YA | 3 |
| | X₁ = G or R | |
| | X₂ = S, Y, or N | |
| | X₃ = S, G, F, or T | |
| HCDR2 amino acid sequence | INNDX₄X₅X₆ST | 4 |
| | _{X}4 = G, I, or V | |
| | _{X}5 = R, V, I, or S | |
| | _{X}6 = S or G | |
| HCDR3 amino acid sequence | ARGGTSGYDT | 5 |
| LCDR1 amino acid sequence | _{X7SVYKNY} | 6 |
| | _{X7 = P or Q} | |
| LCDR2 amino acid sequence | X₈AS | 7 |
| | X₈ = G or K | |
| LCDR3 amino acid sequence | AGGYX₉GX₁₀X₁₁DIFX₁₂ | 8 |
| | X₉ = R or I | |
| | X₁₀ = S or Y | |
| | X₁₁ = S, G, or R | |
| | X₁₂ = A or s | |

TABLE 20
Table 7
CDR Sequences (IMGT definition)-SEQ ID NO 1 Binders

| | | SEQ | | SEQ | | SEQ | | SEQ | | SEQ | | SEQ |
|---|---|---|---|---|---|---|---|---|---|---|---|---|
| Binder | LCDR1 | ID NO: | LCDR2 | ID NO: | LCDR3 | ID NO: | HCDR1 | ID NO: | HCDR2 | ID NO: | HCDR3 | ID NO: |
| BC-B10 | PSVYKNY | 68 | GAS | 70 | AGGYIGSRDIFS | 72 | RFSLSSYA | 76 | INNDVISST | 83 | ARGGTSGYDI | 5 |
| BC-H9 | PSVYKNY | 68 | GAS | 70 | AGGYRGSSDIFA | 73 | GFSLYTYA | 77 | INNDGISST | 84 | ARGGTSGYDI | 5 |
| SC-B6 | PSVYKNY | 68 | GAS | 70 | AGGYIGSRDIFS | 72 | GFSLNNYA | 78 | INNDGISST | 84 | ARGGTSGYDI | 5 |
| SC-F8 | PSVYKNY | 68 | GAS | 70 | AGGYRGSSDIFA | 73 | RFSLYSNYA | 79 | INNDGISST | 84 | ARGGTSGYDI | 5 |
| BC-E1 | PSVYKNY | 68 | GAS | 70 | AGGYRGSSDIFA | 73 | GGSLSSYA | 80 | INNDGRSST | 85 | ARGGTSGYDI | 5 |
| BC-E11 | PSVYKNY | 68 | GAS | 70 | AGGYRGSSDIFA | 73 | GGSLSSYA | 80 | INNDGRSST | 85 | ARGGTSGYDI | 5 |
| BC-E3 | PSVYKNY | 68 | GAS | 70 | AGGYRGSSDIFA | 73 | GGSLSSYA | 80 | INNDGRSST | 85 | ARGGTSGYDI | 5 |
| BC-F2 | PSVYKNY | 68 | GAS | 70 | AGGYRGSSDIFA | 73 | GGSLSSYA | 80 | INNDGRSST | 85 | ARGGTSGYDI | 5 |
| BC-E4 | PSVYKNY | 68 | GAS | 70 | AGGYRGSSDIFA | 73 | GGSLSSYA | 80 | INNDGRSST | 85 | ARGGTSGYDI | 5 |
| SC-D1 | PSVYKNY | 68 | GAS | 70 | AGGYRGSSDIFA | 73 | GGSLSSYA | 80 | INNDGVSST | 86 | ARGGTSGYDI | 5 |
| BC-G6 | PSVYKNY | 68 | GAS | 70 | AGGYRGSSDIFA | 73 | GFSLSGYA | 81 | INNDGISST | 84 | ARGGTSGYDI | 5 |
| BC-A6 | PSVYKNY | 68 | GAS | 70 | AGGYRGSSDIFA | 73 | RFSLSSYA | 76 | INNDGSSST | 87 | ARGGTSGYDI | 5 |

TABLE 21
Table 7
CDR Sequences (IMGT definition)-SEQ ID NO 1 Binders

| | | SEQ | | SEQ | | SEQ | | SEQ | | SEQ | | SEQ |
|---|---|---|---|---|---|---|---|---|---|---|---|---|
| Binder | LCDR1 | ID NO: | LCDR2 | ID NO: | LCDR3 | ID NO: | HCDR1 | ID NO: | HCDR2 | ID NO: | HCDR3 | ID NO: |
| BC-A9 | PSVYKNY | 68 | GAS | 70 | AGGYRGSSDIFA | 73 | RFSLSSYA | 76 | INNDGISST | 84 | ARGGTSGYDI | 5 |
| SC-H10 | PSVYKNY | 68 | GAS | 70 | AGGYRGSSDIFA | 73 | RFSLSSYA | 76 | INNDGISST | 84 | ARGGTSGYDI | 5 |
| BC-V8 | PSVYKNY | 68 | GAS | 70 | AGGYRGSSDIFA | 73 | RFSLSSYA | 76 | INNDGISST | 84 | ARGGTSGYDI | 5 |
| SC-F6 | PSVYKNY | 68 | GAS | 70 | AGGYRGSSDIFA | 73 | RFSLSSYA | 76 | INNDGISST | 84 | ARGGTSGYDI | 5 |
| BC-S3 | PSVYKNY | 68 | GAS | 70 | AGGYRGSSDIFA | 73 | RFSLSSYA | 76 | INNDGVSST | 86 | ARGGTSGYDI | 5 |
| BC-E2 | PSVYKNY | 68 | KAS | 71 | AGGYRGSSDIFA | 73 | RFSLSSYA | 76 | INNDGISST | 84 | ARGGTSGYDI | 5 |
| SC-D10 | PSVYKNY | 68 | KAS | 71 | AGGYRGSSDIFA | 73 | RFSLSSYA | 76 | INNDGVSST | 86 | ARGGTSGYDI | 5 |
| BC-H4 | PSVYKNY | 68 | GAS | 70 | AGGYRGSGDIFA | 74 | RFSLSSYA | 76 | INNDGVSST | 86 | ARGGTSGYDI | 5 |
| BC-B4 | QSVYKNY | 69 | GAS | 70 | AGGYRGSGDIFA | 74 | RFSLSSYA | 76 | INNDGISST | 84 | ARGGTSGYDI | 5 |
| BC-F7 | QSVYKNY | 69 | GAS | 70 | AGGYRGSGDIFA | 74 | RFSLSSYA | 76 | INNDGISST | 84 | ARGGTSGYDI | 5 |
| BC-F4 | PSVYKNY | 68 | GAS | 70 | AGGYRGSSDIFA | 73 | RFSLYSNYA | 79 | INNDGISST | 84 | ARGGTSGYDI | 5 |
| SC-B2 | PSVYKNY | 68 | GAS | 70 | AGGYRGSGDIFA | 74 | RFSLYSNYA | 79 | INNDGISST | 84 | ARGGTSGYDI | 5 |

TABLE 22
Table 7
CDR Sequences (IMGT definition)-SEQ ID NO 1 Binders

| | | SEQ | | SEQ | | SEQ | | SEQ | | SEQ | | SEQ |
|---|---|---|---|---|---|---|---|---|---|---|---|---|
| Binder | LCDR1 | ID NO: | LCDR2 | ID NO: | LCDR3 | ID NO: | HCDR1 | ID NO: | HCDR2 | ID NO: | HCDR3 | ID NO: |
| BC-B8 | PSVYKNY | 68 | GAS | 70 | AGGYRGSSDIFA | 73 | GFSLYTYA | 77 | INNDGISST | 84 | ARGGTSGYDI | 5 |
| BC-S3 | PSVYKNY | 68 | GAS | 70 | AGGYRGSSDIFA | 73 | GFSLYTYA | 77 | INNDGISST | 84 | ARGGTSGYDI | 5 |
| SC-C7 | PSVYKNY | 68 | GAS | 70 | AGGYRGSSDIFA | 73 | RFSLYSGYA | 82 | INNDISGST | 88 | ARGGTSGYDI | 5 |
| SC-G8 | PSVYKNY | 68 | KAS | 71 | AGGYRGYSDIFA | 75 | GFSLSSYA | 80 | INNDGSGST | 89 | ARGGTSGYDI | 5 |

TABLE 23
Table 8

| | | | SEQ ID |
|---|---|---|---|
| Binder | | Sequence | NO: |
| BC-A3 | VH | QTVKESGGRLVTPGAPLTLTCTVSRFSLSSYAMTWVRQAPGKGLEWIG | 95 |
| | | IINNDGVSSTWYASWVKGRFTISKTSTTMDLKMTSLTTEDTATYFCAR | |
| | | GGTSGYDIWGPGTLVTVSL | |
| | VL | AQVLTQTPASVSAAVRGTVTINCQSSPSVYKNYLSWYQQKSGQPPKLL | 119 |
| | | IYGASTLASGVPSRFKGSGSGTQFTLTISDVQCDDAATYYCAGGYRGS | |
| | | SDIFAFGGGTEVVVK | |
| BC-A8 | VH | QTVKESGGRLVTPGAPLTLTCTVSRFSLSSYAMTWVRQAPGKGLEWIG | 120 |
| | | IINNDGSSSTWYASWVKGRFTISKTSTTMDLKMTSLTTEDTATYFCAR | |
| | | GGTSGYDIWGPGTLVTVSL | |
| | VL | AQGLTQTPSPVSAAVGGTVTINCQSSPSVYKNYLSWYQQKPGQPPKLL | 121 |
| | | IYGASTLASGVPSRFKGSGSGTQFTLTISDVQCDDAATYYCAGGYRGS | |
| | | SDIFAFGGGTEVVVK | |
| EC-A9 | VH | QSVEESGGRLVTPGGSLTLTCTVSRFSLSSYAMTWVRQAPGKGLEWIG | 122 |
| | | IINNDGISSTWYASWVKGRFTISKTSTTMDLKMTSLTTEDTATYFCAR | |
| | | GGTSGYDIWGPGTVVTVSL | |
| | VL | AQGLTQTPASVSAAVGGTVTINCQSSPSVYKNYLSWYQQKSGQPPKLL | 14 |
| | | IYGASTLASGAPSRFKGSGSGTQFTLTISDVQCDDAATYYCAGGYRGS | |
| | | SDIFAFGGGTEVVVK | |
| BC-B10 | VH | QSVEESGGRLVTPGAPLTLTCTVSRFSLSSYAMTWVRQAPGKGLEWIG | 15 |
| | | IINNDVISSTWYASWVKGRFTISKTSTTMELKMTSLTTEDTATYFCAR | |
| | | GGTSGYDIWGPGTLVTVSS | |
| | VL | AIKMTQTPSPVSAAVGGTVTINCQSSPSVYKNYLSWYQQKPGQPPKLL | 16 |
| | | IYGASTLASGVPSRFKGSGSGTQFTLTISDVQCDDAATYYCAGGYIGS | |
| | | RDIFSEFGGGTEVVVK | |
| BC-B4 | VH | QSVEESGGRLVTPGGSLTLTCTVSRFSLSSYAMTWVRQAPGKGLEWIG | 17 |
| | | IINNDGISSTWYASWVKGRFTISKTSTTMDLKMTSLTTEDTATYFCAR | |

TABLE 24
Table 8

| | | | SEQ ID |
|---|---|---|---|
| Binder | | Sequence | NO: |
| | | GGTSGYDIWGPGTVVTVSL | |
| | VL | ALVLTQTPSPVSAAVGGIVTINCQSSQSVYKNYLSWYQQKPGQPPKLL | 18 |
| | | IYGASTLASGVPSRFKGSGSGTQFTLTISDVQCDDAATYYCAGGYRGS | |
| | | GDIFAFGGGTEVVVK | |
| BC-B8 | VH | QSLEESGGRLVTPGTPLTLTCTVSGFSLYTYAMGWVRQAPGKGLEWIG | 19 |
| | | IINNDGISSTWYASWVKGRFTISKTSTTMDLKMTSLTTEDTATYFCAR | |
| | | GGTSGYDIWGPCTLVTVSL | |
| | VL | AQGLTQTPSPVSAAVGGTVTINCQSGPSVYKNYLSWYQQKAGQPPKLL | 20 |
| | | IYGASTLASGVPSRFKGSGSGTQFTLTISDVQCDDAATYYCAGGYRGS | |
| | | SDIFAFGGGTEVVVK | |
| BC-C8 | VH | QSLEESGGRLVTPGGSLTLTCTVSRFSLSSYAMTWVRQAPGKGLEWIG | 21 |
| | | IINNDGISSTWYASWVKGRFTISKTSTTMDLKMTSLTTEDTATYFCAR | |
| | | GGTSGYDIWGPGTVVTVSL | |
| | VL | ALVLTQTPSPVSAAVGGTVTINCQSSPSVYKNYLSWYQQKPGQPPKLL | 22 |
| | | IYGASTLASGVPSRFKGSGSGTQFTLTISDVQCDDAATYYCAGGYRGS | |
| | | SDIFAFGGGTEVVVK | |
| BC-D3 | VH | QSLEESGGRLVTPGTPLTLTCTVSGFSLYTYAMGWVRQAPGKGLEWIG | 23 |
| | | IINNDGISSTWYASWVKGRFTISKTSTTMDLKMTSLTTEDTATYFCAR | |
| | | GGTSGYDIWGPGTLVTVSL | |
| | VL | AQGLTQTPSPVSAAVGGTVTINCQSGPSVYKNYLSWYQQKAGQPPKLL | 24 |
| | | IYGASTLASGVPSRFKGSGSGTQFTLTISDVQCDDAATYYCAGGYRGS | |
| | | SDIFAFGGGTEVVVK | |
| BC-E1 | VH | QSVKESGGRLVAPGTPLTLTCAVSGFSLSSYAMTWVRQAPGKGLEWIG | 25 |
| | | IINNDGRSSTWYASWVKGRFTISKTSTTMDLKMTSPTTEDTATYFCAR | |
| | | GGTSGYDIWGPGTLVTVSL | |
| | VL | AQGMTQTPSPVSAAVGGTVTINCQSSPSVYKNYLSWYQQKPGQPPKLL | 26 |

TABLE 25
Table 8

| | | | SEQ ID |
|---|---|---|---|
| Binder | | Sequence | NO: |
| | | IYGASTLASGVPSRFKGSGSGTRFTLTISDVQCDDAATYYTAGGYRGS | |
| | | SDIFAFGGGTEVVVK | |
| BC-E11 | VH | QSVKESGGRLVAPGTPLTLTCAVSGFSLSSYAMTWVRQAPGKGLEWIG | 27 |
| | | IINNDGRSSTWYASWVKGRFTISKTSTTMDLKMTSPTTEDTATYFCAR | |
| | | GGTSGYDIWGPGTLVTVSL | |
| | VL | AQGMTQTPSPVSAAVGGTVTINCQSSPSVYKNYLSWYQQKPGQPPKLL | 28 |
| | | IYGASTLASGVPSRFKGSGSGTRFTLTISDVQCDDAATYYCAGGYRGS | |
| | | SDIFAFGGGTEVVVK | |
| BC-E2 | VH | QEQLKESGGRLVTPGAPLTLTCTVSRFSLSSYAMTWVRQAPGKGLEWI | 29 |
| | | GIINNDGISSTWYASWVKGRFTISKTSTTMDLKMTSLTTEDTATYFCA | |
| | | RGGTSGYDIWGPGTLVTVSL | |
| | VL | AVVLTQTPSPVSAAVGGTVTINCQSSPSVYKNYLSWYQQKPGQPPKLL | 30 |
| | | IYKASTLASGVPSRFKGSGSGTQFTLTISDVQCDDAATYYCAGGYRGS | |
| | | SDIFAFGGGTEVVVK | |
| BC-E3 | VH | QSVKESGGRLVAPGTPLTLTCAVSGFSLSSYAMTWVRQAPGKGLEWIG | 31 |
| | | IINNDGRSSTWYASWVKGRFTISKTSTTMDLKMTSPTTEDTATYFCAR | |
| | | GGTSGYDIWGPGTLVTVSL | |
| | VL | AQGMTQTPSPVSAAVGGTVTINCQSSPSVYKNYLSWYQQKPGQPPKLL | 32 |
| | | IYGASTLASGVPSRFKGSGSGTRFTLTISDVQCDDAATYYCAGGYRGS | |
| | | SDIFAFGCGTEVVVIK | |
| BC-E4 | VH | QSVKESGGRLVAPGTPLTLTCAVSGFSLSSYAMTWVRQAPGKGLEWIG | 33 |
| | | IINNDGRSSTWYASWVKGRFTISKTSTTMDLKMTSPTTEDTATYFCAR | |
| | | GGTSGYIDIWGPGTLVTVSL | |
| | VL | AQGMTQTPSPVSAAVGGTVTINCQSSPSVYKNYLSWYQQKPGQPPKLL | 34 |
| | | IYGASTLASGVPSRFKGSGSGTRFTLTISDVQCDDAATYYCAGGYRGS | |
| | | SDIFAFGGGTEVVVK | |

TABLE 26
Table 8

| | | | SEQ ID |
|---|---|---|---|
| Binder | | Sequence | NO: |
| BC-F2 | VH | QSVKESGGRLVAPGTPLTLTCAVSGFSLSSYAMTWVRQAPGKGLEWIG | 35 |
| | | IIDDGRSSTWYASWVKGRFTISKTSTTMDLKMTSPTTEDTATYFACAR | |
| | | GGTSGYDIWGPGTLVTVSL | |
| | VL | AQGMTQTPSPVSAAVGGTVTINCQSSPSVYKNYLSWYQQKPGQPPKLL | 36 |
| | | IYGASTLASGVPSRFKGSGSGTRFTLTISDVQCDDAATYYCAGGYRGS | |
| | | SDIFAFGGGTEVVVK | |
| BC-F4 | VH | QSVEESRGRLVTPGGSLTLTCTVSRFSLSNYAMTWVRQAFGKGLEWIG | 37 |
| | | IINNDGISSTWYASWVKGRFTISKTSTTMDLKMTSLTTEDTATYFCAR | |
| | | GGTSGYDIWGPGTLVTVSL | |
| | VL | AQVLTQTPSPVSAAVGGTVTINCQSSPSVYKNYLSWYQQKPGQPPKLL | 38 |
| | | IYGASTLASGVPSRFKGSGSGTQFTLTISDVQCDDAATYYCAGGYRGS | |
| | | SDIFAFGGGTEVVVK | |
| BC-F7 | VH | QSVEESGGRLVTPGGSLTLTCTVSRFSLSSYAMTWVRQAPGKGLEWIG | 39 |
| | | IINNDGISSTWYASWVKGRFTISKTSTTMDLKMTSLTTEDTATYFCAR | |
| | | GGTSGYDIWGPGTVVTVSL | |
| | VL | QLVLTQTPSPVSAAVGGTVTINCQSSQSVYKNYLSWYQQKPGQPPKLL | 40 |
| | | IYGASTLASGVPSRFKGSGSGTQFTLTISDVQCDDAATYYCAGGYRGS | |
| | | GDIFAFGGGTEVVVK | |
| BC-G6 | VH | QSVEESGGRLVTPGGSLTLTCTASGFSLSGYAMTWVRQAPGKGLEWIG | 41 |
| | | IINNDGISSTWYASWVKGRFTISKTSTTMDLKMTSLTTEDTATYFCAR | |
| | | GGTSGYDIWGPGTLVTVSL | |
| | VL | ALVMTQTPSPVSAAVGGTVTINCQSSPSVYKNYLSWYQQKPGQPPKLL | 42 |
| | | IYGASTLASGVPSRFKGSGSGTQFTLTISDVQCDDAATYYCAGGYRGS | |
| | | SDIFAFGGGTEVVVK | |
| BC-H4 | VH | QQLKESGGRLVTPGAPLTLTCTVSRFSLSSYAMTWVRQAPGKGLEWIG | 43 |
| | | IINNDGVSSTWYASWVKGRFTISKTSTTMDLKMTSLTTEDTATYFCAR | |

TABLE 27
Table 8

| | | | SEQ ID |
|---|---|---|---|
| Binder | | Sequence | NO: |
| | | GGTSGYDIWGPGTLVTVSS | |
| | VL | ALVLTQTPSPVSAAVGGTVTINCQSSPSVYKNYLSWYQQKPGQPPKLL | 44 |
| | | IYGASTLASGVPSRFKGSGSGTQFTLTISDVQCDDAATYYCAGGYRGS | |
| | | GDIFAFGGGTEVVVK | |
| BC-H9 | VH | QSLEESGGRLVTPGTPLTLTCTVSGFSLYTYAMGWVRQAPGKGLEWIG | 45 |
| | | IINNDGISSTWYASWVKGRFTISKTSTTMDLKMTSLTTEDTATYFCAR | |
| | | GGTSGYDIWGPGTLVTVSL | |
| | VL | AQGLTQTPSPVSAAVGGTVTINCQSGPSVYKNYLSWYQQKAGQPPKLL | 46 |
| | | IYGASTLASGVPSRFKGSGSGTQFTLTISDVQCDDAATYYCAGGYRGS | |
| | | SDIFAFGGGTEVVVK | |
| SC-B2 | VH | QEQLMESRGRLVTPGGSLTLTCTVSRFSLSNYAMTWVRQAPGKGLEWI | 47 |
| | | GIINNDGISSTWYASWVKGRFTISKTSTTMDLKMTSLTTEDTATYFCA | |
| | | RGGTSGYDIWGPGTLVTVSL | |
| | VL | AQGPTQTPSPVSAAVGGTVTINCQSSPSVYKNYLSWYQQKPGQPPKLL | 48 |
| | | IYGASTLASGVPSRFKGSGSGTQFTLTISDVQCDDAATYYCAGGYRGS | |
| | | GDIFAFGGGTEVVVK | |
| SC-B6 | VH | QSVKESGGRLVTPGAPLTLTCTVSGFSLNNYAMTWVRQAPGKGLEWIG | 49 |
| | | IINNDGISSTWYASWVKGRFIISKTSTTVDLKMTSLTTEDTATYFCAR | |
| | | GGTSGYDIWGPGTLVTVSL | |
| | VL | AQGPTQTPSPVSAAVGGTVTINCQSSPSVYKNYLSWYQQKPGQPPKLL | 50 |
| | | IYGASTLASGVPSRFKGSRSGTQFTLTISDVQCDDAATYYCAGGYIGS | |
| | | RDIFSFGGGTEVVVK | |
| SC-C7 | VH | QTVKESGGRLVTPGAPLTLTCTVSRFSLSGYAMTWVRQAPGKGLEWIG | 51 |
| | | IINNDISGSTWYASWVKGRFTISKTSTTMDLKMTSLTTEDTATYFCAR | |
| | | GGTSGYDIWGPGTLVTVSS | |
| | VL | DVVMTQTPSPVSAAVGGTVTINCQSSPSVYKNYLSWYQQKPGQPPKLL | 52 |

TABLE 28
Table 8

| | | | SEQ ID |
|---|---|---|---|
| Binder | | Sequence | NO: |
| | | IYGASTLASGVPSRFKGSGSGTQFTLTISDVQCDDAATYYCAGGYRGS | |
| | | SDIFAFGGGTEVVVK | |
| SC-D1 | VH | QSLEESRGRLVTPGGSLTLTCTVSGFSLSSYAMTWVRQAPGKGLEWIG | 53 |
| | | IINNDGVSSTWYASWVKGRFTISKTSTTMDLKMTSLTTEDTATYVCAR | |
| | | GGTSGYDIWGPGTLVTVSL | |
| | VL | DPMMTQTPSPVSAAVGGTVTINCQSSPSVYKNYLSWYQQKPGQPPKLL | 54 |
| | | IYGASTLASGVPSRFKGSGSGTQFTLTISGVQCDDAATYYCAGGYRGS | |
| | | SDIFAFGGGTEVVVK | |
| SC-D10 | VH | QSVKESGGRLVTPGGSLTLTCTVSRFSLSSYAMTWVRQAPGKGLEWIG | 55 |
| | | IINNDGVSSTWYASWVKGRFTISKTSTTMDLKMTSLTTEDTATYFCAR | |
| | | GGTSGYDIWGPGTLVTVSL | |
| | VL | ALVMTQTPSPVSAAVGGTVTTNCQSSPSVYKNYLSWYQQKPGQPPKLL | 56 |
| | | IYKASTLASGVPSRFSGSGSGTQFTLTISDVQCDDAATYYCAGGYRGS | |
| | | SDIFAFGGGTEVVVK | |
| SC-F6 | VH | QSVKESGGRLVTPGAPLTLTCTVSRFSLSSYAMTWVRQAPGKGLEWIG | 57 |
| | | IINNDGISSTWYASWVKGRFTISKTSTTMDLKMTSLTTEDTATYFCAR | |
| | | GGTSGYDIWGPGTLVTVSL | |
| | VL | AQGMTQTPASVSAAVGGTVTINCQSSPSVYKNYLSWYQQKSGQPPKLL | 58 |
| | | IYGASTLASGVPSRFKGSGSGTQFTLTISDVQCDDAATYYCAGGYRGS | |
| | | SDIFAFGGGTEVVVK | |
| SC-F8 | VH | QTVKESGGRLVTPGGSLTLTCTVSRFSLSNYAMTWVRQAPGKGLEWIG | 59 |
| | | IINNDGISSTWYASWVKGRFTISKTSTTMDLKMTSLTTEDTATYFCAR | |
| | | GGTSGYDIWGPGTLVTVSL | |
| | VL | AQVLTQTPSPVSAAVGGTVTINCQSSPSVYKNYLSWYQQKPGQPPKLL | 60 |
| | | IYGASTLASGVPSRFKGSGSGTQFTLTISDVQCDDAATYYCAGGYRGS | |
| | | SDIFAFGGGTEVVVK | |

TABLE 29
Table 8

| | | | SEQ ID |
|---|---|---|---|
| Binder | | Sequence | NO: |
| SC-G8 | VH | QSVEESGGRLVMPGGSLTLTCTASGFSLSSYAMTWVRQAPGKGLEWIG | 61 |
| | | IINNDGSGSTWYASWVKGRFTISKTSTTMDLKMTSLTTEDTATYFCAR | |
| | | GGTSGYDIWGPGTLVTVSL | |
| | VL | AAVLTQTPSPVSAAVGGTVTINCQSSPSVYKNYLSWYQQKPGQPPKLL | 62 |
| | | IYKASTLASGVPSRFKGSGSGTQFTLTISDVQCDDAATYYCAGGYRGY | |
| | | SDIFAFGGGTEVVVK | |
| SC-H10 | VH | QSLEESGGRLVTPGAPLTLTCTVSRFSLSSYAMTWVRQAPGKGLEWIG | 63 |
| | | IINNDGISSTWYASWVKGRFTISKTSTTMKLKMTSLTTEDTATYFCAR | |
| | | GGTSGYDIWGPGTLVTVSL | |
| | VL | AQGLTQTPSPVSAAVGGTVTINCQSSPSVYKNYLSWYQQKPGQPPKLL | 64 |
| | | IYGASTLASGVPSRFSGSGSGTQFTLTISDVQCDDAATYYCAGGYRGS | |
| | | SDIFAFGGGTEVVVK | |

### Specific Embodiments

While various specific embodiments have been illustrated and described, it will be appreciated that various changes can be made without departing from the spirit and scope of the disclosure(s). The present disclosure is exemplified by the numbered embodiments set forth below.
1. An anti-AQP3 antibody or a functional fragment thereof comprising:
(a) a heavy chain complementarity determining region 1 (HCDR1) comprising the amino acid sequence X₁FSLX₂X₃YA (SEQ ID NO:3), where X₁ is G or R, X₂ is S, Y, or N, and X₃ is S, G, N, or T;
(b) a heavy chain complementarity determining region 2 (HCRD2) comprising the amino acid sequence INNDX₄X₅X₆ST (SEQ ID NO:4), where X₄ is G, I, or V, X₅ is R, V, I, or S, and X₆ is S or G;
(c) a heavy chain complementarity determining region 3 (HCDR3) comprising the amino acid sequence ARGGTSGYDI (SEQ ID NO:5);
(d) a light chain complementarity determining region 1 (LCDR1) comprising the amino acid sequence X₇SVYKNY (SEQ ID NO:6), where X₇ is P or Q;
(e) a light chain complementarity determining region 2 (LCDR2) comprising the amino acid sequence X₈AS (SEQ ID NO:7), where X₈ is G or K; and
(f) a light chain complementarity determining region 3 (LCDR3) comprising the amino acid sequence AGGYX₉GX₁₀X₁₁DIFX₁₂ (SEQ ID NO:8), where X₉ is R or I, X₁₀ is S or Y, X₁₁ is S, G, or R, and X₁₂ is A or S.
2. The anti-AQP3 antibody or a functional fragment thereof of embodiment 1, wherein X₁ is G.
3. The anti-AQP3 antibody or a functional fragment thereof of embodiment 1, wherein X₁ is R.
4. The anti-AQP3 antibody or a functional fragment thereof of any one of embodiments 1 to 3, wherein X₂ is S.
5. The anti-AQP3 antibody or a functional fragment thereof of any one of embodiments 1 to 3, wherein X₂ is Y.
6. The anti-AQP3 antibody or a functional fragment thereof of any one of embodiments 1 to 3, wherein X₂ is N.
7. The anti-AQP3 antibody or a functional fragment thereof of any one of embodiments 1 to 6, wherein X₃ is S.
8. The anti-AQP3 antibody or a functional fragment thereof of any one of embodiments 1 to 6, wherein X₃ is G.
9. The anti-AQP3 antibody or a functional fragment thereof of any one of embodiments 1 to 6, wherein X₃ is N.
10. The anti-AQP3 antibody or a functional fragment thereof of any one of embodiments 1 to 6, wherein X₃ is T.
11. The anti-AQP3 antibody or a functional fragment thereof of any one of embodiments 1 to 10, wherein X₄ is G.
12. The anti-AQP3 antibody or a functional fragment thereof of any one of embodiments 1 to 10, wherein X₄ is I.
13. The anti-AQP3 antibody or a functional fragment thereof of any one of embodiments 1 to 10, wherein X₄ is V.
14. The anti-AQP3 antibody or a functional fragment thereof of any one of embodiments 1 to 13, wherein X₅ is R.
15. The anti-AQP3 antibody or a functional fragment thereof of any one of embodiments 1 to 13, wherein X₅ is V.
16. The anti-AQP3 antibody or a functional fragment thereof of any one of embodiments 1 to 13, wherein X₅ is I.
17. The anti-AQP3 antibody or a functional fragment thereof of any one of embodiments 1 to 13, wherein X₅ is S.
18. The anti-AQP3 antibody or a functional fragment thereof of any one of embodiments 1 to 17, wherein X₆ is S.
19. The anti-AQP3 antibody or a functional fragment thereof of any one of embodiments 1 to 17, wherein X₆ is G.
20. The anti-AQP3 antibody or a functional fragment thereof of any one of embodiments 1 to 19, wherein X₇ is P.
21. The anti-AQP3 antibody or a functional fragment thereof of any one of embodiments 1 to 19, wherein X₇ is Q.
22. The anti-AQP3 antibody or a functional fragment thereof of any one of embodiments 1 to 21, wherein X₈ is G.
23. The anti-AQP3 antibody or a functional fragment thereof of any one of embodiments 1 to 21, wherein X₈ is K.
24. The anti-AQP3 antibody or a functional fragment thereof of any one of embodiments 1 to 23, wherein X₉ is R.
25. The anti-AQP3 antibody or a functional fragment thereof of any one of embodiments 1 to 23, wherein X₉ is I.
26. The anti-AQP3 antibody or a functional fragment thereof of any one of embodiments 1 to 25, wherein X₁₀ is S.
27. The anti-AQP3 antibody or a functional fragment thereof of any one of embodiments 1 to 25, wherein X₁₀ is Y.
28. The anti-AQP3 antibody or a functional fragment thereof of any one of embodiments 1 to 27, wherein X₁₁ is S.
29. The anti-AQP3 antibody or a functional fragment thereof of any one of embodiments 1 to 27, wherein X₁₁ is G.
30. The anti-AQP3 antibody or a functional fragment thereof of any one of embodiments 1 to 27, wherein X₁₁ is R.
31. The anti-AQP3 antibody or a functional fragment thereof of any one of embodiments 1 to 30, wherein X₁₂ is A.
32. The anti-AQP3 antibody or a functional fragment thereof of any one of embodiments 1 to 30, wherein X₁₂ is S.
33. An anti-AQP3 antibody or a functional fragment thereof comprising:
(a) a heavy chain complementarity determining region 1 (HCDR1) comprising the amino acid sequence X₁₃FSLX₁₄X₁₅YA (SEQ ID NO:9), where X₁₃ is G or R, X₁₄ is S, Y, or N, and X₁₅ is S, N, or T;
(b) a heavy chain complementarity determining region 2 (HCRD2) comprising the amino acid sequence INNDX₁₆ISST (SEQ ID NO:10), where X₁₆ is G or V;
(c) a heavy chain complementarity determining region 3 (HCDR3) comprising the amino acid sequence ARGGTSGYDI (SEQ ID NO:5);
(d) a light chain complementarity determining region 1 (LCDR1) comprising the amino acid sequence PSVYKNY (SEQ ID NO:11);
(e) a light chain complementarity determining region 2 (LCDR2) comprising the amino acid sequence GAS (SEQ ID NO:12); and
(f) and a light chain complementarity determining region 3 (LCDR3) comprising the amino acid sequence AGGYX₁₇GSX₁₈DIFX₁₉ (SEQ ID NO:13), where X₁₇ is R or I X₁₈ is S or R, and X₁₉ is A or S.
34. The anti-AQP3 antibody or a functional fragment thereof of embodiment 33, wherein X₁₃ is G.
35. The anti-AQP3 antibody or a functional fragment thereof of embodiment 33, wherein X₁₃ is R.
36. The anti-AQP3 antibody or a functional fragment thereof of any one of embodiments 33 to 35, wherein X₁₄ is S.
37. The anti-AQP3 antibody or a functional fragment thereof of any one of embodiments 33 to 35, wherein X₁₄ is Y.
38. The anti-AQP3 antibody or a functional fragment thereof of any one of embodiments 33 to 35, wherein X₁₄ is N.
39. The anti-AQP3 antibody or a functional fragment thereof of any one of embodiments 33 to 38, wherein X₁₅ is S.
40. The anti-AQP3 antibody or a functional fragment thereof of any one of embodiments 33 to 38, wherein X₁₅ is N.
41. The anti-AQP3 antibody or a functional fragment thereof of any one of embodiments 33 to 38, wherein X₁₅ is T.
42. The anti-AQP3 antibody or a functional fragment thereof of any one of embodiments 33 to 41, wherein X₁₆ is G.
43. The anti-AQP3 antibody or a functional fragment thereof of any one of embodiments 33 to 41, wherein X₁₆ is V.
44. The anti-AQP3 antibody or a functional fragment thereof of any one of embodiments 33 to 43, wherein X₁₇ is R.
45. The anti-AQP3 antibody or a functional fragment thereof of any one of embodiments 33 to 43, wherein X₁₇ is I.
46. The anti-AQP3 antibody or a functional fragment thereof of any one of embodiments 33 to 45, wherein X₁₈ is S.
47. The anti-AQP3 antibody or a functional fragment thereof of any one of embodiments 33 to 45, wherein X₁₈ is R.
48. The anti-AQP3 antibody or a functional fragment thereof of any one of embodiments 33 to 47, wherein X₁₉ is A.
49. The anti-AQP3 antibody or a functional fragment thereof of any one of embodiments 33 to 47, wherein X₁₉ is S.
50. The anti-AQP3 antibody or a functional fragment thereof of embodiment 1, which comprises the HCDR1, HCDR2, HCDR3, LCDR1, LCDR2, and LCDR3 sequences of one of the binders set forth in Table 7.
51. The anti-AQP3 antibody or a functional fragment thereof of embodiment 50, which comprises the HCDR1, HCDR2, HCDR3, LCDR1, LCDR2, and LCDR3 sequences of BC—B10 as set forth in Table 7.
52. The anti-AQP3 antibody or a functional fragment thereof of embodiment 50, which comprises the HCDR1, HCDR2, HCDR3, LCDR1, LCDR2, and LCDR3 sequences of BC—H9 as set forth in Table 7.
53. The anti-AQP3 antibody or a functional fragment thereof of embodiment 50, which comprises the HCDR1, HCDR2, HCDR3, LCDR1, LCDR2, and LCDR3 sequences of SC—B6 as set forth in Table 7.
54. The anti-AQP3 antibody or a functional fragment thereof of embodiment 50, which comprises the HCDR1, HCDR2, HCDR3, LCDR1, LCDR2, and LCDR3 sequences of SC—F8 as set forth in Table 7.
55. The anti-AQP3 antibody or a functional fragment thereof of embodiment 1, comprising variable heavy (VH) and variable light (VL) chain sequences of one of the binders set forth in Table 8.
56. The anti-AQP3 antibody or a functional fragment thereof of embodiment 55, which comprises the VH and VL sequences of BC—B10.
57. The anti-AQP3 antibody or a functional fragment thereof of embodiment 55, which comprises the VH and VL sequences of BC—H9.
58. The anti-AQP3 antibody or a functional fragment thereof of embodiment 55, which comprises the VH and VL sequences of SC—B6.
59. The anti-AQP3 antibody or a functional fragment thereof of embodiment 55, which comprises the VH and VL sequences of SC—F8.
60. An anti AQP3 antibody or a functional fragment thereof that specifically binds to an oligopeptide whose amino acid sequence comprises or consists of ATYPSGHLDM (SEQ ID NO:1), SGPNGTAGIFATYPS (SEQ ID NO:94); YPSGH (SEQ ID NO:90); PS (SEQ ID NO:93); or GHLDM (SEQ ID NO:91).
61. The anti AQP3 antibody or a functional fragment thereof of any one of embodiments 1 to 60, which specifically binds to an oligopeptide whose amino acid sequence consists of ATYPSGHLDM (SEQ ID NO:1)
62. The anti AQP3 antibody or a functional fragment thereof of any one of embodiments 1 to 61, which does not comprise a heavy chain (HC) and a light chain (LC) comprising any of the HC and LC sequences set forth in Table 1.
63. The anti AQP3 antibody or a functional fragment thereof of any one of embodiments 1 to 61, which does not comprise HCDR1, HCDR2, HCDR3, LCDR1, LCDR2, and LCDR3 sequences of any one of antibodies A, B, C, D, E, F, G, H, J, and K as defined by IMGT (Lefranc et al., 2003, Dev Comparat Immunol 27:55-77), Kabat (Kabat et al., 1991, Sequences of Proteins of Immunological Interest, 5th Ed. Public Health Service, National Institutes of Health, Bethesda, Md.) or Chothia (A1-Lazikani et al., 1997, J. Mol. Biol 273:927-948).
64. The anti AQP3 antibody or a functional fragment thereof of any one of embodiments 1 to 61, which is not an antibody or functional fragment described in PCT/JP2018/038220, the contents of which are incorporated herein by reference in their entirety.
65. The anti AQP3 antibody or a functional fragment thereof of any one of embodiments 1 to 61, which does not comprise HCDR1, HCDR2, HCDR3, LCDR1, LCDR2, and LCDR3 sequences comprising the HCDR1, HCDR2, HCDR3, LCDR1, LCDR2, and LCDR3 sequences set forth in any of Tables 2A-2J.
66. The anti AQP3 antibody or a functional fragment thereof of any one of embodiments 1 to 61, which does not comprise a VH sequence and VL sequence comprising the VH and VL sequences set forth in any of Tables 2A-2J.
67. The anti AQP3 antibody or a functional fragment thereof of any one of embodiments 1 to 61, which does not comprise a HCDR2 sequence comprising X₁DPEX₂ GGT (SEQ ID NO: 225), where X₁ is V or I and X₂ is T or S
68. The anti AQP3 antibody or a functional fragment thereof of any one of embodiments 1 to 61, which does not comprise a HCDR2 sequence comprising GX₁ DPEX₂GGTX₃YNQKFX₄G (SEQ ID NO: 229), where X₁ is V or I, X₂=T or S, X₃=G or A, and X₄=R or K;
69. The anti AQP3 antibody or a functional fragment thereof of any one of embodiments 1 to 68, which does not comprise a HCDR2 sequence comprising DPEX₁ GG (SEQ ID NO: 230), where X₁ is T or S;
70. The anti AQP3 antibody or a functional fragment thereof of any one of embodiments 1 to 69, which does not comprise a HCDR2 sequence comprising ISRX₁ SIYT (SEQ ID NO: 231), where X₁ is G or R;
71. The anti AQP3 antibody or a functional fragment thereof of any one of embodiments 1 to 70, which does not comprise a HCDR2 sequence comprising TISRX₁ SIYTYYPDSVX₂G (SEQ ID NO: 232), where X₁ is G or R and X₂ is K or Q;
72. The anti AQP3 antibody or a functional fragment thereof of any one of embodiments 1 to 71, which does not comprise a HCDR2 sequence comprising SRX₁SIY (SEQ ID NO: 233), where X₁ is G or R;
73. The anti AQP3 antibody or a functional fragment thereof of any one of embodiments 1 to 72, which does not comprise a HCDR2 sequence comprising IX₁ PGSGX₂T (SEQ ID NO: 234), where X₁ is Y or F and X₂ is N or S;
74. The anti AQP3 antibody or a functional fragment thereof of any one of embodiments 1 to 73, which does not comprise a HCDR2 sequence comprising X₁IX₂ PGSGX₃TYYNEKX₄KG (SEQ ID NO: 235), where X₁ is E or W, X₂ is Y or F, X₃ is N or S, and X₄ is L or F.
75. The anti AQP3 antibody or a functional fragment thereof of any one of embodiments 1 to 74, which specifically binds to at least one of the oligopeptides whose amino acid sequence is set forth in Table 4.
76. The anti AQP3 antibody or a functional fragment thereof of embodiment 75, which exhibits a binding signal which is at least 25% of the binding signal of a rabbit IgG antibody comprising the VH and VL of antibody SC—F8, a rabbit IgG antibody comprising the VH and VL of a rabbit IgG antibody comprising the VH and VL of antibody BC—B10 and/or a rabbit IgG antibody comprising the VH and VL of antibody SC—B6 in the ELISA assay of Example 16 when performed with a peptide of SEQ ID NO:94.
77. The anti AQP3 antibody or a functional fragment thereof of embodiment 75, which exhibits a binding signal which is at least 50% of the binding signal of a rabbit IgG antibody comprising the VH and VL of antibody SC—F8, a rabbit IgG antibody comprising the VH and VL of antibody BC—B10 and/or a rabbit IgG antibody comprising the VH and VL of antibody SC—B6 in the ELISA assay of Example 16 when performed with a peptide of SEQ ID NO:94.
78. The anti AQP3 antibody or a functional fragment thereof of embodiment 75, which exhibits a binding signal which is at least 75% of the binding signal of a rabbit IgG antibody comprising the VH and VL of antibody SC—F8, a rabbit IgG antibody comprising the VH and VL of antibody BC—B10 and/or a rabbit IgG antibody comprising the VH and VL of antibody SC—B6 in the ELISA assay of Example 16 when performed with a peptide of SEQ ID NO:94.
79. The anti AQP3 antibody or a functional fragment thereof of any one of embodiments 75 to 78, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:1
80. The anti AQP3 antibody or a functional fragment thereof of any one of embodiments 75 to 78, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:1.
81. The anti AQP3 antibody or a functional fragment thereof of any one of embodiments 75 to 78, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:1.
82. The anti AQP3 antibody or a functional fragment thereof of any one of embodiments 75 to 81, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:96.
83. The anti AQP3 antibody or a functional fragment thereof of any one of embodiments 75 to 81, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:96.
84. The anti AQP3 antibody or a functional fragment thereof of any one of embodiments 75 to 81, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:96.
85. The anti AQP3 antibody or a functional fragment thereof of any one of embodiments 75 to 84, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:97.
86. The anti AQP3 antibody or a functional fragment thereof of any one of embodiments 75 to 84, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:97.
87. The anti AQP3 antibody or a functional fragment thereof of any one of embodiments 75 to 84, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:97.
88. The anti AQP3 antibody or a functional fragment thereof of any one of embodiments 75 to 87, which exhibits a binding signal which is at least 25% of the binding signal of a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:98.
89. The anti AQP3 antibody or a functional fragment thereof of any one of embodiments 75 to 87, which exhibits a binding signal which is at least 50% of the binding signal of a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:98.
90. The anti AQP3 antibody or a functional fragment thereof of any one of embodiments 75 to 87, which exhibits a binding signal which is at least 75% of the binding signal of a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:98.
91. The anti AQP3 antibody or a functional fragment thereof of any one of embodiments 75 to 90, which exhibits a binding signal which is at least 25% of the binding signal of a rabbit IgG antibody comprising the VH and VL of antibody SC—B10 in the ELISA assay of Example 16 when performed with a peptide of SEQ ID NO:99.
92. The anti AQP3 antibody or a functional fragment thereof of any one of embodiments 75 to 90, which exhibits a binding signal which is at least 50% of the binding signal of a rabbit IgG antibody comprising the VH and VL of antibody SC—B10 in the ELISA assay of Example 16 when performed with a peptide of SEQ ID NO:99.
93. The anti AQP3 antibody or a functional fragment thereof of any one of embodiments 75 to 90, which exhibits a binding signal which is at least 75% of the binding signal of a rabbit IgG antibody comprising the VH and VL of antibody SC—B10 in the ELISA assay of Example 16 when performed with a peptide of SEQ ID NO:99.
94. The anti AQP3 antibody or a functional fragment thereof of any one of embodiments 75 to 93, which exhibits a binding signal which is at least 25% of the binding signal of a rabbit IgG antibody comprising the VH and VL of antibody SC—B10 in the ELISA assay of Example 16 when performed with a peptide of SEQ ID NO:100.
95. The anti AQP3 antibody or a functional fragment thereof of any one of embodiments 75 to 93, which exhibits a binding signal which is at least 50% of the binding signal of a rabbit IgG antibody comprising the VH and VL of antibody SC—B10 in the ELISA assay of Example 16 when performed with a peptide of SEQ ID NO:100.
96. The anti AQP3 antibody or a functional fragment thereof of any one of embodiments 75 to 93, which exhibits a binding signal which is at least 75% of the binding signal of a rabbit IgG antibody comprising the VH and VL of antibody SC—B10 in the ELISA assay of Example 16 when performed with a peptide of SEQ ID NO:100.
97. The anti AQP3 antibody or a functional fragment thereof of any one of embodiments 75 to 96, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:101.
98. The anti AQP3 antibody or a functional fragment thereof of any one of embodiments 75 to 96, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:101.
99. The anti AQP3 antibody or a functional fragment thereof of any one of embodiments 75 to 96, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:101.
100. The anti AQP3 antibody or a functional fragment thereof of any one of embodiments 75 to 99, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:102.
101. The anti AQP3 antibody or a functional fragment thereof of any one of embodiments 75 to 99, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:102.
102. The anti AQP3 antibody or a functional fragment thereof of any one of embodiments 75 to 99, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:102.
103. The anti AQP3 antibody or a functional fragment thereof of any one of embodiments 75 to 102, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:103.
104. The anti AQP3 antibody or a functional fragment thereof of any one of embodiments 75 to 102, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:103.
105. The anti AQP3 antibody or a functional fragment thereof of any one of embodiments 75 to 102, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, and/or a rabbit IgG antibody comprising the VH and VL of antibody SC—B6 in the ELISA assay of Example 16 when performed with a peptide of SEQ ID NO:103.
106. The anti AQP3 antibody or a functional fragment thereof of any one of embodiments 75 to 105, which exhibits a binding signal which is at least 25% of the binding signal of a rabbit IgG antibody comprising the VH and VL of antibody BC—B10 in the ELISA assay of Example 16 when performed with a peptide of SEQ ID NO:104.
107. The anti AQP3 antibody or a functional fragment thereof of any one of embodiments 75 to 105, which exhibits a binding signal which is at least 50% of the binding signal of a rabbit IgG antibody comprising the VH and VL of antibody BC—B10 in the ELISA assay of Example 16 when performed with a peptide of SEQ ID NO:104.
108. The anti AQP3 antibody or a functional fragment thereof of any one of embodiments 75 to 105, which exhibits a binding signal which is at least 75% of the binding signal of a rabbit IgG antibody comprising the VH and VL of antibody BC—B10 in the ELISA assay of Example 16 when performed with a peptide of SEQ ID NO:104.
109. The anti AQP3 antibody or a functional fragment thereof of any one of embodiments 75 to 108, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:105.
110. The anti AQP3 antibody or a functional fragment thereof of any one of embodiments 75 to 108, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:105.
111. The anti AQP3 antibody or a functional fragment thereof of any one of embodiments 75 to 108, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:105.
112. The anti AQP3 antibody or a functional fragment thereof of any one of embodiments 75 to 111, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:106.
113. The anti AQP3 antibody or a functional fragment thereof of any one of embodiments 75 to 111, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:106.
114. The anti AQP3 antibody or a functional fragment thereof of any one of embodiments 75 to 111, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:106.
115. The anti AQP3 antibody or a functional fragment thereof of any one of embodiments 75 to 114, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:107.
116. The anti AQP3 antibody or a functional fragment thereof of any one of embodiments 75 to 114, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:107.
117. The anti AQP3 antibody or a functional fragment thereof of any one of embodiments 75 to 114, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:107.
118. The anti AQP3 antibody or a functional fragment thereof of any one of embodiments 75 to 117, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or v SC—B10 in the ELISA assay of Example 16 when performed with a peptide of SEQ ID NO:108.
119. The anti AQP3 antibody or a functional fragment thereof of any one of embodiments 75 to 117, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:108.
120. The anti AQP3 antibody or a functional fragment thereof of any one of embodiments 75 to 117, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:108.
121. The anti AQP3 antibody or a functional fragment thereof of any one of embodiments 75 to 120, which exhibits a binding signal which is at least 25% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:109.
122. The anti AQP3 antibody or a functional fragment thereof of any one of embodiments 75 to 120, which exhibits a binding signal which is at least 50% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:109.
123. The anti AQP3 antibody or a functional fragment thereof of any one of embodiments 75 to 120, which exhibits a binding signal which is at least 75% of the binding signal of a rabbit IgG antibody comprising the VH and VL of antibody SC—F8, a rabbit IgG antibody comprising the VH and VL of antibody BC—H9, a rabbit IgG antibody comprising the VH and VL of antibody BC—B10, a rabbit IgG antibody comprising the VH and VL of antibody SC—B6 and/or a rabbit IgG antibody comprising the VH and VL of antibody SC—B10 in the ELISA assay of Example 16 when performed with a peptide of SEQ ID NO:109.
124. The anti AQP3 antibody or a functional fragment thereof of any one of embodiments 75 to 123, which specifically binds to the oligopeptide SGPNG-TAGIFATYPS (SEQ ID NO:94).
125. The anti AQP3 antibody or a functional fragment thereof of any one of embodiments 75 to 124, which specifically binds to the oligopeptide ATYPSGHLDM (SEQ ID NO:1).
126. The anti AQP3 antibody or a functional fragment thereof of any one of embodiments 75 to 125, which specifically binds to the oligopeptide TYPSGHLDM (SEQ ID NO:96).
127. The anti AQP3 antibody or a functional fragment thereof of any one of embodiments 75 to 126, which specifically binds to a the oligopeptide YPSGHLDM (SEQ ID NO:97).
128. The anti AQP3 antibody or a functional fragment thereof of any one of embodiments 75 to 127, which specifically binds to the oligopeptide PSGHLDM (SEQ ID NO:98).
129. The anti AQP3 antibody or a functional fragment thereof of any one of embodiments 75 to 128, which specifically binds to the oligopeptide SGHLDM (SEQ ID NO:99).
130. The anti AQP3 antibody or a functional fragment thereof of any one of embodiments 75 to 129, which specifically binds to the oligopeptide GHLDM (SEQ ID NO:100).
131. The anti AQP3 antibody or a functional fragment thereof of any one of embodiments 75 to 130, which specifically binds to the oligopeptide ATYPSGHLD (SEQ ID NO:101).
132. The anti AQP3 antibody or a functional fragment thereof of any one of embodiments 75 to 131, which specifically binds to the oligopeptide ATYPSGHL (SEQ ID NO:102).
133. The anti AQP3 antibody or a functional fragment thereof of any one of embodiments 75 to 132, which specifically binds to the oligopeptide ATYPSGH (SEQ ID NO:103).
134. The anti AQP3 antibody or a functional fragment thereof of any one of embodiments 75 to 133, which specifically binds to the oligopeptide ATYPSG (SEQ ID NO:104).
135. The anti AQP3 antibody or a functional fragment thereof of any one of embodiments 75 to 134, which specifically binds to the oligopeptide TAGI-FATYPSGHLDM (SEQ ID NO:105).
136. The anti AQP3 antibody or a functional fragment thereof of any one of embodiments 75 to 135, which specifically binds to the oligopeptide AGI-FATYPSGHLDM (SEQ ID NO:106).
137. The anti AQP3 antibody or a functional fragment thereof of any one of embodiments 75 to 136, which specifically binds to the oligopeptide GIFATYPSGHLDM (SEQ ID NO:107).
138. The anti AQP3 antibody or a functional fragment thereof of any one of embodiments 75 to 137, which specifically binds to the oligopeptide IFATYPSGHLDM (SEQ ID NO:108).
139. The anti AQP3 antibody or a functional fragment thereof of any one of embodiments 75 to 138, which specifically binds to the oligopeptide FATYPSGHLDM (SEQ ID NO:109).
140. The anti AQP3 antibody or a functional fragment thereof of any one of embodiments 1 to 139 that specifically binds to human and/or mouse AQP3.
141. The anti AQP3 antibody or a functional fragment thereof of any one of embodiments 1 to 140 that specifically binds to the extracellular portion of human and/or mouse AQP3.
142. The anti AQP3 antibody or a functional fragment thereof of embodiment 141, which specifically binds to the extracellular portion of cell surface expressed human and/or mouse AQP3.
143. The anti AQP3 antibody or a functional fragment thereof of embodiment 142, which specifically binds to the extracellular portion of human AQP3 expressed on the surface of HaCaT cells and/or the extracellular portion of mouse AQP3 expressed on the surface of PAM212 cells.
144. The anti AQP3 antibody or a functional fragment thereof of any of embodiments 1 to 143, wherein the antibody or functional fragment thereof binds to SEQ ID NO:1 with an affinity greater than 100 pM.
145. The anti AQP3 antibody or a functional fragment thereof of any of embodiments 1 to 144, wherein the antibody or functional fragment thereof binds to Loop C of human and/or mouse AQP3 with an affinity greater than 100 pM.
146. The anti AQP3 antibody or a functional fragment thereof of any of embodiments 1 to 145, wherein the antibody or functional fragment thereof binds to human and/or mouse AQP3 with an affinity greater than 100 pM.
147. The anti AQP3 antibody or a functional fragment thereof of any of embodiments 1 to 146, which is antibody.
148. The anti AQP3 antibody or a functional fragment thereof of any one of embodiments 1 to 147, which is a IgG antibody.
149. The anti AQP3 antibody or a functional fragment thereof of any one of embodiments 1 to embodiment 148, which is a IgG antibody comprising murine Fc sequences.
150. The anti AQP3 antibody or a functional fragment thereof of any one of embodiments 1 to embodiment 148, which is a IgG antibody comprising human Fc sequences.
151. The anti AQP3 antibody or a functional fragment thereof of any one of embodiments 1 to embodiment 148, which is a IgG antibody comprising rabbit Fc sequences.
152. An anti AQP3 antibody or a functional fragment thereof that competes with the antibody or functional fragment thereof according to any one of embodiments 1 to 151 for binding to an oligopeptide whose amino acid sequence comprises or consists of SEQ ID NO:1.
153. An anti AQP3 antibody or a functional fragment thereof that competes with the antibody or functional fragment thereof according to any one of embodiments 1 to 151 for binding to Loop C of human AQP3.
154. An anti AQP3 antibody or a functional fragment thereof that competes with the antibody or functional fragment thereof according to any one of embodiments 1 to 151 for binding to Loop C of mouse AQP3.
155. An anti AQP3 antibody or a functional fragment thereof that competes with the antibody or functional fragment thereof according to any one of embodiments 1 to 151 for binding to human AQP3.
156. The anti AQP3 antibody or a functional fragment thereof of embodiment 155, wherein the competition is for binding to cell surface expressed human AQP3.
157. The anti AQP3 antibody or a functional fragment thereof of embodiment 156, wherein the competition is for binding to human AQP3 expressed on the surface of HaCaT cells.
158. An anti AQP3 antibody or a functional fragment thereof that competes with the antibody or functional fragment thereof according to any one of embodiments 1 to 151 for binding to mouse AQP3.
159. The anti AQP3 antibody or a functional fragment thereof of embodiment 158, wherein the competition is for binding to cell surface expressed mouse AQP3.
160. The anti AQP3 antibody or a functional fragment thereof of embodiment 159, wherein the competition is for binding to mouse AQP3 expressed on the surface of PAM212 cells.
161. The anti AQP3 antibody or a functional fragment thereof of any of embodiments 1 to 160, wherein the antibody or functional fragment thereof has an inhibitory activity on at least one function of human and/or mouse AQP3.
162. The anti AQP3 antibody or a functional fragment thereof of any of embodiments 1 to 161, wherein the inhibitory activity of at least one function of human and/or mouse AQP3 comprises reduction in H₂O₂ transport.
163. The anti AQP3 antibody or a functional fragment thereof of any of embodiments 1 to 162, wherein the inhibitory activity of at least one function of human and/or mouse AQP3 comprises at least a 50% reduction in H₂O₂ transport.
164. The anti AQP3 antibody or a functional fragment thereof of any of embodiments 162 to 163, wherein reduction in H₂O₂ transport is measured according to the assay described in Example 5.
165. An anti AQP3 antibody or a functional fragment thereof that specifically binds to ATYPSGHLDM (SEQ ID NO:1), wherein the antibody or functional fragment thereof inhibits a functional response of keratinoid cells that are dependent on transport of H₂O ₂.
166. The anti AQP3 antibody or a functional fragment thereof of embodiment 165, wherein the antibody or functional fragment thereof inhibits functional responses of keratinoid cells that are dependent on transport of H₂O₂ by at least 50%.
167. An anti AQP3 antibody or a functional fragment thereof that specifically binds to ATYPSGHLDM (SEQ ID NO:1), wherein the antibody or functional fragment thereof inhibits functional responses of immune cells that are dependent on transport of H₂O₂.
168. The anti AQP3 antibody or a functional fragment thereof of embodiment 167, wherein the antibody or functional fragment thereof inhibits functional responses of immune cells that are dependent on transport of H₂O₂ by at least 50%.
169. The anti AQP3 antibody or a functional fragment thereof of any one of embodiments 167-168, wherein the immune cells are selected from macrophages and T-cells.
170. An anti AQP3 antibody or a functional fragment thereof that specifically binds to Loop C of human AQP3, wherein the antibody or functional fragment thereof has an inhibitory activity on at least one function of human and/or mouse AQP3.
171. The anti AQP3 antibody or a functional fragment thereof of embodiment 170, wherein the inhibitory activity of at least one function of human and/or mouse AQP3 comprises reduction in H₂O₂ transport.
172. The anti AQP3 antibody or a functional fragment thereof of any of embodiments 170-171, wherein the inhibitory activity of at least one function of human and/or mouse AQP3 comprises at least a 50% reduction in H₂O₂ transport.
173. The anti AQP3 antibody or a functional fragment thereof of any of embodiments 171-172, wherein reduction in H₂O₂ transport is measured according to the assay described in Example 5.
174. An anti AQP3 antibody or a functional fragment thereof that specifically binds to Loop C of human AQP3, wherein the antibody or functional fragment thereof inhibits a functional response of keratinoid cells that are dependent on transport of H₂O₂.
175. The anti AQP3 antibody or a functional fragment thereof of embodiment 174, wherein the antibody or functional fragment thereof inhibits functional responses of keratinoid cells that are dependent on transport of H₂O₂ by at least 50%.
176. An anti AQP3 antibody or a functional fragment thereof that specifically binds to Loop C of human AQP3, wherein the antibody or functional fragment thereof inhibits functional responses of immune cells that are dependent on transport of H₂O₂.
177. The anti AQP3 antibody or a functional fragment thereof of embodiment 176, wherein the antibody or functional fragment thereof inhibits functional responses of immune cells that are dependent on transport of H₂O₂ by at least 50%.
178. The anti AQP3 antibody or a functional fragment thereof of any one of embodiments 176-177, wherein the immune cells are selected from macrophages and T-cells.
179. An antibody drug conjugate (ADC) comprising the anti AQP3 antibody or a functional fragment thereof according to any one of embodiments 1 to 178 conjugated to a cytotoxic agent
180. The ADC of embodiment 179, wherein the cytotoxic agent comprises an alkylating agent.
181. The ADC of embodiment 180, wherein the alkylating agent comprises cisplatin, carboplatin, oxaliplatin, mechlorethamine, cyclophosphamide, melphalan, chlo-rambucil, ifosfamide busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), procarbazine or hexamethylmelamine.
182. The ADC of embodiment 179, wherein the cytotoxic agent comprises an antimetabolite.
183. The ADC of embodiment 182, wherein the antimetabolite comprises methotrexate, pemetrexed, capecitabine, cytarabine, gemcitabine, decitabine, azacitidine, fludarabine, nelarabine, cladribine, clofarabine, or pentostatin.
184. The ADC of embodiment 179, wherein the cytotoxic agent comprises an antimicrotubule agent.
185. The ADC of embodiment 184, wherein the antimicrotubule agent comprises pa-clitaxel, docetaxel, vincristine, vinorelbine, vinblastine, vindesine, vinflunine, monomethyl auristatin E, or monomethyl auristatin F.
186. The ADC of embodiment 179, wherein the cytotoxic agent comprises a topoisomerase inhibitor.
187. The ADC of embodiment 186, wherein the topoisomerase inhibitor comprises irinotecan, topotecan, etoposide, doxorubicin, mitoxantrone, teniposide, novobiocin, merbarone, or aclarubicin.
188. The ADC of embodiment 179, wherein the cytotoxic agent comprises a cytotoxic antibiotic.
189. The ADC of embodiment 188, wherein the cytotoxic antibiotic comprises doxorubicin, daunorubicin, epirubicin idarubicin, pirarubicin, aclarubicin, mitoxantrone, or bleomycin.
190. A method of treating a subject having cancer comprising administering a therapeutically effective amount of the anti AQP3 antibody or functional fragment thereof according to any one of embodiments 1 to 178 or the ADC of any one of embodiments 179 to 189 to the subject.
191. The method according to embodiment 190, wherein the cancer is cancer selected from the group consisting of colorectal cancer, cervical cancer, liver cancer, lung cancer, esophageal cancer, kidney cancer, stomach cancer, tongue cancer, skin cancer, and breast cancer.
192. A method of preventing and/or treating a skin disorder in a subject comprising administering a therapeutically effective amount of the antibody or functional fragment thereof according to any one of embodiments 1 to 178 to the subject.
193. The method of embodiment 192, wherein the skin disorder is selected from the group consisting of psoriasis, actinic keratosis, ichthyosis, and seborrheic dermatitis.
194. The method of embodiment 192 or embodiment 193, which is a method of treating a skin disorder.
195. A method of preventing and/or treating an inflammatory disorder in a subject comprising administering a therapeutically effective amount of the antibody or functional fragment thereof according to any one of embodiments 1 to 178 to the subject.
196. The method according to embodiment 195, wherein the inflammatory disorder is selected from the group consisting of atopic dermatitis, psoriasis, asthma, chronic obstructive pulmonary disease, and hepatitis.
197. The method of embodiment 195 or embodiment 196, which is a method of treating an inflammatory disorder
198. A method for producing an anti AQP3 antibody comprising steps of a) injecting an animal with SEQ ID NO:1; b) collecting one or more organs from the animal containing cells that produce antibodies; c) isolating mRNA from the organs; d) creating an antibody phage library using the mRNA; and e) screening the antibody phage library created in step d) to identify one or more antibodies that bind to SEQ ID NO:1.
199. The method of embodiment 198, wherein the animal is a mouse.
200. The method of embodiment 198, wherein the animal is a rabbit.
201. The method of any one of embodiments 198 to 200, wherein the organs are selected from spleen and bone marrow.
202. A method for inhibiting at least one function of AQP3 comprising a step of contacting an AQP3 containing sample with an anti AQP3 antibody or a functional fragment thereof that specifically binds to SEQ ID NO:1, optionally wherein the antibody or functional fragment there is an antibody of functional thereof according to any one of embodiments 1 to 178.
203. A method for inhibiting at least one function of AQP3 comprising a step of contacting an AQP3 containing sample with an anti AQP3 antibody or a functional fragment thereof that specifically binds to Loop C of human AQP3, optionally wherein the antibody or functional fragment there is an antibody of functional thereof according to any one of embodiments 1 to 178.
204. A method for inhibiting at least one function of AQP3 comprising a step of contacting an AQP3 containing sample with an anti AQP3 antibody or a functional fragment thereof that specifically binds to the extracellular portion of human AQP3, optionally wherein the antibody or functional fragment there is an antibody of functional thereof according to any one of embodiments 1 to 178.
205. A method for inhibiting transport of H₂O₂ across a membrane comprising a step of contacting a sample having a membrane including AQP3 with an anti AQP3 antibody or a functional fragment thereof that specifically binds to SEQ ID NO:1, optionally wherein the antibody or functional fragment there is an antibody of functional thereof according to any one of embodiments 1 to 178.
206. A method for inhibiting transport of H₂O₂ across a membrane comprising a step of contacting a sample having a membrane including AQP3 with an anti AQP3 antibody or a functional fragment thereof that specifically binds to the extracellular portion of human AQP3, optionally wherein the antibody or functional fragment there is an antibody of functional thereof according to any one of embodiments 1 to 178.
207. A method for inhibiting transport of H₂O₂ across a membrane comprising a step of contacting a sample having a membrane including AQP3 with an anti AQP3 antibody or a functional fragment thereof that specifically binds to Loop C of human AQP3, optionally wherein the antibody or functional fragment there is an antibody of functional thereof according to any one of embodiments 1 to 178.
208. A method for separating and/or purifying AQP3-expressing cells comprising a step of contacting a sample including cells with an anti AQP3 antibody or a functional fragment thereof that specifically binds to SEQ ID NO:1, optionally wherein the antibody or functional fragment there is an antibody of functional thereof according to any one of embodiments 1 to 178.
209. A method for separating and/or purifying AQP3-expressing cells comprising a step of contacting a sample including cells with an anti AQP3 antibody or a functional fragment thereof that specifically binds to Loop C of human AQP3, optionally wherein the antibody or functional fragment there is an antibody of functional thereof according to any one of embodiments 1 to 178.
210. A method for separating and/or purifying AQP3-expressing cells comprising a step of contacting a sample including cells with an anti AQP3 antibody or a functional fragment thereof that specifically binds to the extracellular portion of human AQP3, optionally wherein the antibody or functional fragment there is an antibody of functional thereof according to any one of embodiments 1 to 178
211. A method for measuring AQP3 comprising a step of contacting a sample with an anti AQP3 antibody or a functional fragment thereof that specifically binds to SEQ ID NO:1, optionally wherein the antibody or functional fragment there is an antibody of functional thereof according to any one of embodiments 1 to 178.
212. A method for measuring AQP3 comprising a step of contacting a sample with an anti AQP3 antibody or a functional fragment thereof that specifically binds to the Loop C of human AQP3, optionally wherein the antibody or functional fragment there is an antibody of functional thereof according to any one of embodiments 1 to 178.
213. A method for measuring AQP3 comprising a step of contacting a sample with an anti AQP3 antibody or a functional fragment thereof that specifically binds to the extracellular portion of human AQP3, optionally wherein the antibody or functional fragment there is an antibody of functional thereof according to any one of embodiments 1 to 178.

1. An anti-AQP3 antibody or a functional fragment thereof comprising:
(a) a heavy chain complementarity determining region 1 (HCDR1) comprising the amino acid sequence X₁FSLX₂X₃YA (SEQ ID NO:3), where X₁ is G or R, X₂ is S, Y, or N, and X₃ is S, G, N, or T;
(b) a heavy chain complementarity determining region 2 (HCRD2) comprising the amino acid sequence INNDX₄X₅X₆ST (SEQ ID NO:4), where X₄ is G, I, or V, X₅ is R, V, I, or S, and X₆ is S or G;
(c) a heavy chain complementarity determining region 3 (HCDR3) comprising the amino acid sequence ARGGTSGYDI (SEQ ID NO:5);
(d) a light chain complementarity determining region 1 (LCDR1) comprising the amino acid sequence X₇SVYKNY (SEQ ID NO:6), where X₇ is P or Q;
(e) a light chain complementarity determining region 2 (LCDR2) comprising the amino acid sequence X₈AS (SEQ ID NO:7), where X₈ is G or K; and
(f) a light chain complementarity determining region 3 (LCDR3) comprising the amino acid sequence AGGYX₉GX₁₀X₁₁DIFX₁₂ (SEQ ID NO:8), where X₉ is R or I, X₁₀ is S or Y, X₁₁ is S, G, or R, and X₁₂ is A or S.

2. An anti-AQP3 antibody or a functional fragment thereof comprising:
(a) a heavy chain complementarity determining region 1 (HCDR1) comprising the amino acid sequence X₁₃FSLX₁₄X₁₅YA (SEQ ID NO:9), where X₁₃ is G or R, X₁₄ is S, Y, or N, and X₁₅ is S, N, or T;
(b) a heavy chain complementarity determining region 2 (HCRD2) comprising the amino acid sequence INNDX₁₆ISST (SEQ ID NO:10), where X₁₆ is G or V;
(c) a heavy chain complementarity determining region 3 (HCDR3) comprising the amino acid sequence ARGGTSGYDI (SEQ ID NO:5);
(d) a light chain complementarity determining region 1 (LCDR1) comprising the amino acid sequence PSVYKNY (SEQ ID NO:11);
(e) a light chain complementarity determining region 2 (LCDR2) comprising the amino acid sequence GAS (SEQ ID NO:12); and
(f) a light chain complementarity determining region 3 (LCDR3) comprising the amino acid sequence AGGYX₁₇GSX₁₈DIFX₁₉ (SEQ ID NO:13), where X₁₇ is R or I, X₁₈ is S or R, and X₁₉ is A or S.

3. The anti-AQP3 antibody or a functional fragment thereof of claim 1, which comprises the HCDR1, HCDR2, HCDR3, LCDR1, LCDR2, and LCDR3 sequences of one of the binders set forth in Table 7.

4. The anti-AQP3 antibody or a functional fragment thereof of claim 3, which comprises the HCDR1, HCDR2, HCDR3, LCDR1, LCDR2, and LCDR3 sequences of BC—B10, BC—H9, SC—B6, or SC—F8 as set forth in Table 7.

5. The anti-AQP3 antibody or a functional fragment thereof of claim 1, comprising variable heavy (VH) and variable light (VL) chain sequences of one of the binders set forth in Table 8.

6. The anti-AQP3 antibody or a functional fragment thereof of claim 5, which comprises the VH and VL sequences of BC—B10, BC—H9, SC—B6, or SC—F8.

7. An anti AQP3 antibody or a functional fragment thereof that specifically binds to an oligopeptide whose amino acid sequence comprises or consists of ATYPSGHLDM (SEQ ID NO:1), SGPNG-TAGIFATYPS (SEQ ID NO:94), YPSGH (SEQ ID NO:90), PS (SEQ ID NO:93), or GHLDM (SEQ ID NO:91).

8. The anti AQP3 antibody or a functional fragment thereof of any one of claims 1 to 7, which specifically binds to an oligopeptide whose amino acid sequence consists of ATYPSGHLDM (SEQ ID NO:1).

9. The anti AQP3 antibody or a functional fragment thereof of any one of claims 1 to 8, which specifically binds to at least one of the oligopeptides whose amino acid sequence is set forth in Table 4.

10. The anti AQP3 antibody or a functional fragment thereof of any one of claims 1 to 9 that specifically binds to human and/or mouse AQP3.

11. The anti AQP3 antibody or a functional fragment thereof of any one of claims 1 to 10 that specifically binds to the extracellular portion of human and/or mouse AQP3.

12. The anti AQP3 antibody or a functional fragment thereof of claim 11, which specifically binds to the extracellular portion of cell surface expressed human and/or mouse AQP3.

13. The anti AQP3 antibody or a functional fragment thereof of claim 12, which specifically binds to the extracellular portion of human AQP3 expressed on the surface of HaCaT cells and/or the extracellular portion of mouse AQP3 expressed on the surface of PAM212 cells.

14. The anti AQP3 antibody or a functional fragment thereof of any one of claims 1 to 13, wherein the antibody or functional fragment thereof binds to SEQ ID NO:1 with an affinity greater than 100 pM.

15. The anti AQP3 antibody or a functional fragment thereof of any one of claims 1 to 14, wherein the antibody or functional fragment thereof binds to Loop C of human and/or mouse AQP3 with an affinity greater than 100 pM.

16. The anti AQP3 antibody or a functional fragment thereof of any one of claims 1 to 15, wherein the antibody or functional fragment thereof binds to human and/or mouse AQP3 with an affinity greater than 100 pM.

17. An anti AQP3 antibody or a functional fragment thereof that competes with the antibody or functional fragment thereof according to any one of claims 1 to 16 for binding to an oligopeptide whose amino acid sequence comprises or consists of SEQ ID NO:1.

18. An anti AQP3 antibody or a functional fragment thereof that competes with the antibody or functional fragment thereof according to any one of claims 1 to 17 for binding to Loop C of human AQP3.

19. An anti AQP3 antibody or a functional fragment thereof that competes with the antibody or functional fragment thereof according to any one of claims 1 to 18 for binding to Loop C of mouse AQP3.

20. An anti AQP3 antibody or a functional fragment thereof that competes with the antibody or functional fragment thereof according to any one of claim 19 for binding to human AQP3.

21. The anti AQP3 antibody or a functional fragment thereof of claim 20, wherein the competition is for binding to cell surface expressed human AQP3.

22. The anti AQP3 antibody or a functional fragment thereof of claim 21, wherein the competition is for binding to human AQP3 expressed on the surface of HaCaT cells.

23. An anti AQP3 antibody or a functional fragment thereof that competes with the antibody or functional fragment thereof according to any one of claims 1 to 22 for binding to mouse AQP3.

24. The anti AQP3 antibody or a functional fragment thereof of claim 23, wherein the competition is for binding to cell surface expressed mouse AQP3.

25. The anti AQP3 antibody or a functional fragment thereof of claim 24, wherein the competition is for binding to mouse AQP3 expressed on the surface of PAM212 cells.

26. The anti AQP3 antibody or a functional fragment thereof of any one of claims 1 to 25, wherein the antibody or functional fragment thereof has an inhibitory activity on at least one function of human and/or mouse AQP3.

27. The anti AQP3 antibody or a functional fragment thereof of any one of claims 1 to 26, wherein the inhibitory activity of at least one function of human and/or mouse AQP3 comprises reduction in H₂O₂ transport.

28. An anti AQP3 antibody or a functional fragment thereof that specifically binds to ATYPSGHLDM (SEQ ID NO:1), wherein the antibody or functional fragment thereof inhibits a functional response of keratinoid cells that are dependent on transport of H₂O₂.

29. An anti AQP3 antibody or a functional fragment thereof that specifically binds to ATYPSGHLDM (SEQ ID NO:1), wherein the antibody or functional fragment thereof inhibits functional responses of immune cells that are dependent on transport of H₂O₂.

30. An anti AQP3 antibody or a functional fragment thereof that specifically binds to Loop C of human AQP3, wherein the antibody or functional fragment thereof has an inhibitory activity on at least one function of human and/or mouse AQP3.

31. The anti AQP3 antibody or a functional fragment thereof of claim 30, wherein the inhibitory activity of at least one function of human and/or mouse AQP3 comprises reduction in H₂O₂ transport.

32. An anti AQP3 antibody or a functional fragment thereof that specifically binds to Loop C of human AQP3, wherein the antibody or functional fragment thereof inhibits a functional response of keratinoid cells that are dependent on transport of H₂O₂.

33. An anti AQP3 antibody or a functional fragment thereof that specifically binds to Loop C of human AQP3, wherein the antibody or functional fragment thereof inhibits functional responses of immune cells that are dependent on transport of H₂O₂.

34. An antibody drug conjugate (ADC) comprising the anti AQP3 antibody or a functional fragment thereof according to any one of claims 1 to 33 conjugated to a cytotoxic agent.

35. A method of treating a subject having cancer comprising administering a therapeutically effective amount of the anti AQP3 antibody or functional fragment thereof according to any one of claims 1 to 33 or the ADC of claim 34 to the subject.

36. A method of preventing and/or treating a skin disorder in a subject comprising administering a therapeutically effective amount of the antibody or functional fragment thereof according to any one of claims 1 to 33 to the subject.

37. A method of preventing and/or treating an inflammatory disorder in a subject comprising administering a therapeutically effective amount of the antibody or functional fragment thereof according to any one of claims 1 to 33 to the subject.

38. A method for producing an anti AQP3 antibody comprising steps of a) injecting an animal with SEQ ID NO:1; b) collecting one or more organs from the animal containing cells that produce antibodies; c) isolating mRNA from the organs; d) creating an antibody phage library using the mRNA; and e) screening the antibody phage library created in step d) to identify one or more antibodies that bind to SEQ ID NO:1.

39. A method for inhibiting at least one function of AQP3 comprising a step of contacting an AQP3 containing sample with an anti AQP3 antibody or a functional fragment thereof that specifically binds to SEQ ID NO:1, optionally wherein the antibody or functional fragment there is an antibody of functional thereof according to any one of claims 1 to 33.

40. A method for inhibiting at least one function of AQP3 comprising a step of contacting an AQP3 containing sample with an anti AQP3 antibody or a functional fragment thereof that specifically binds to Loop C of human AQP3, optionally wherein the antibody or functional fragment there is an antibody of functional thereof according to any one of claims 1 to 33.

41. A method for inhibiting at least one function of AQP3 comprising a step of contacting an AQP3 containing sample with an anti AQP3 antibody or a functional fragment thereof that specifically binds to the extracellular portion of human AQP3, optionally wherein the antibody or functional fragment there is an antibody of functional thereof according to any one of claims 1 to 33.

42. A method for inhibiting transport of H₂O₂ across a membrane comprising a step of contacting a sample having a membrane including AQP3 with an anti AQP3 antibody or a functional fragment thereof that specifically binds to SEQ ID NO:1, optionally wherein the antibody or functional fragment there is an antibody of functional thereof according to any one of claims 1 to 33.

43. A method for inhibiting transport of H₂O₂ across a membrane comprising a step of contacting a sample having a membrane including AQP3 with an anti AQP3 antibody or a functional fragment thereof that specifically binds to the extracellular portion of human AQP3, optionally wherein the antibody or functional fragment there is an antibody of functional thereof according to any one of claims 1 to 33.

44. A method for inhibiting transport of H₂O₂ across a membrane comprising a step of contacting a sample having a membrane including AQP3 with an anti AQP3 antibody or a functional fragment thereof that specifically binds to Loop C of human AQP3, optionally wherein the antibody or functional fragment there is an antibody of functional thereof according to any one of claims 1 to 33.

45. A method for separating and/or purifying AQP3-expressing cells comprising a step of contacting a sample including cells with an anti AQP3 antibody or a functional fragment thereof that specifically binds to SEQ ID NO:1, optionally wherein the antibody or functional fragment there is an antibody of functional thereof according to any one of claims 1 to 33.

46. A method for separating and/or purifying AQP3-expressing cells comprising a step of contacting a sample including cells with an anti AQP3 antibody or a functional fragment thereof that specifically binds to Loop C of human AQP3, optionally wherein the antibody or functional fragment there is an antibody of functional thereof according to any one of claims 1 to 33.

47. A method for separating and/or purifying AQP3-expressing cells comprising a step of contacting a sample including cells with an anti AQP3 antibody or a functional fragment thereof that specifically binds to the extracellular portion of human AQP3, optionally wherein the antibody or functional fragment there is an antibody of functional thereof according to any one of claims 1 to 33.

48. A method for measuring AQP3 comprising a step of contacting a sample with an anti AQP3 antibody or a functional fragment thereof that specifically binds to SEQ ID NO:1, optionally wherein the antibody or functional fragment there is an antibody of functional thereof according to any one of claims 1 to 33.

49. A method for measuring AQP3 comprising a step of contacting a sample with an anti AQP3 antibody or a functional fragment thereof that specifically binds to the Loop C of human AQP3, optionally wherein the antibody or functional fragment there is an antibody of functional thereof according to any one of claims 1 to 33.

50. A method for measuring AQP3 comprising a step of contacting a sample with an anti AQP3 antibody or a functional fragment thereof that specifically binds to the extracellular portion of human AQP3, optionally wherein the antibody or functional fragment there is an antibody of functional thereof according to any one of claims 1 to 33.
